(19) United States
(12) Patent Application Publication
(10) Pub. No.: US 2003/0194375 A1
(43) Pub. Date: Oct. 16, 2003

(54) Anti-epileptogenic agents

(75) Inventors: Donald F. Weaver, Halifax (CA); Christopher Y.K. Tan, North York (CA); Stephen T. Kim, Kingston (CA); Xianqi Kong, Dollard-des-Ormeaux (CA); Lan Wei, Edison, NJ (US); John R. Carran, Kingston (CA)

Correspondence Address: LAHIVE & COCKFIELD, 28 STATE STREET, BOSTON, MA, 02109, US

(73) Assignee: Queen's University at Kingston and Neurochem, Inc.

(21) Appl. No.: 10/272,249

(22) Filed: Oct. 15, 2002

Related U.S. Application Data
(63) Continuation of application No. 10/099,934, filed on Mar. 13, 2002.
(60) Provisional application No. 60/275,618, filed on Mar. 13, 2001.

(51) Int. Cl. A61K 49/00; G06G 7/48; G06G 7/58; A61K 31/195; A61K 31/198; A61K 31/165

(52) U.S. Cl. 424/9.2; 703/11; 514/567; 514/620; 514/564

(57) ABSTRACT

Methods and compounds useful for the inhibition of convulsive disorders, including epilepsy, are disclosed. The methods and compounds of the invention inhibit or prevent ictogenesis and/or epileptogenesis. Methods for preparing the compounds of the invention are also described.

### RELATED APPLICATIONS

This application claims the priority of U.S. Provisional Application No. 60/275,618, filed Mar. 13, 2001; and this application is related to and discloses material in addition to U.S. application Ser. No. 09/041,371, filed Mar. 11, 1998, now U.S. Pat. No. 6,306,909, the entire contents of which are incorporated herein by reference.

### BACKGROUND OF THE INVENTION

Epilepsy is a serious neurological condition, associated with seizures, that affects hundreds of thousands of people worldwide. Clinically, a seizure results from a sudden electrical discharge from a collection of neurons in the brain. The resulting nerve cell activity is manifested by symptoms such as uncontrollable movements.

A seizure is a single discrete clinical event caused by an excessive electrical discharge from a collection of neurons through a process termed “ictogenesis.” As such, a seizure is merely the symptom of epilepsy. Epilepsy is a dynamic and often progressive process characterized by an underlying sequence of pathological transformations whereby normal brain is altered, becoming susceptible to recurrent seizures through a process termed “epileptogenesis.” While it is believed that ictogenesis and epileptogenesis have certain biochemical pathways in common, the two processes are not identical. Ictogenesis (the initiation and propagation of a seizure in time and space) is a rapid and definitive electrical/chemical event occurring over seconds or minutes. Epileptogenesis (the gradual process whereby normal brain is transformed into a state susceptible to spontaneous, episodic, time-limited, recurrent seizures, through the initiation and maturation of an “epileptogenic focus”) is a slow biochemical and/or histological process which generally occurs over months to years. Epileptogenesis is a two phase process. Phase 1 epileptogenesis is the initiation of the epileptogenic process prior to the first seizure, and is often the result of stroke, disease (e.g., meningitis), or trauma, such as an accidental blow to the head or a surgical procedure performed on the brain. Phase 2 epileptogenesis refers to the process during which a brain that is already susceptible to seizures, becomes still more susceptible to seizures of increasing frequency and/or severity. While the processes involved in epileptogenesis have not been definitively identified, some researchers believe that upregulation of excitatory coupling between neurons, mediated by N-methyl-D-aspartate (NMDA) receptors, is involved. Other researchers implicate downregulation of inhibitory coupling between neurons, mediated by gamma-amino-butyric acid (GABA) receptors.

Although epileptic seizures are rarely fatal, large numbers of patients require medication to avoid the disruptive, and potential dangerous, consequences of seizures. In many cases, medication is required for extended periods of time, and in some cases, a patient must continue to take prescription drugs for life. Furthermore, drugs used for the management of epilepsy have side effects associated with prolonged usage, and the cost of the drugs can be considerable.

A variety of drugs are available for the management of epileptic seizures, including older anticonvulsant agents such as phenytoin, valproate and carbamazepine (ion channel blockers), as well as newer agents like felbamate, gabapentin, and tiagabine. β-Alanine has been reported to have anticonvulsant activity, as well as NMDA inhibitory activity and GABAergic stimulatory activity, but has not been employed clinically. Currently available accepted drugs for epilepsy are anticonvulsant agents, where the term “anticonvulsant” is synonymous with “anti-seizure” or “anti-ictogenic”; these drugs can suppress seizures by blocking ictogenesis, but it is believed that they do not influence epilepsy because they do not block epileptogenesis. Thus, despite the numerous drugs available for the treatment of epilepsy (i.e., through suppression of the convulsions associated with epileptic seizures), there are no generally accepted drugs for the treatment of the pathological changes which characterize epileptogenesis. There is no generally accepted method of inhibiting the epileptogenic process and there are no generally accepted drugs recognized as anti-epileptogenic.

### SUMMARY OF THE INVENTION

This invention relates to methods and compounds, e.g., anti-ictogenic and/or anti-epileptogenic compounds, useful for the treatment and/or prevention of convulsive disorders including epilepsy.

In one aspect, the invention provides a method for inhibiting epileptogenesis in a subject. The method includes administering to a subject in need thereof an effective amount of an agent which modulates a process in a pathway associated with epileptogenesis such that epileptogenesis is inhibited in the subject.

In another aspect, a method for inhibiting epileptogenesis in a subject is provided. An effective amount of an agent which antagonizes an NMDA receptor and augments endogenous GABA inhibition is administered to a subject in need thereof, such that epileptogenesis is inhibited in the subject. In preferred embodiments, the agent antagonizes an NMDA receptor by binding to the glycine binding site of the NMDA receptors. In preferred embodiments, the agent augments GABA inhibition by decreasing glial GABA uptake. In certain preferred embodiments, the agent comprises a pharmacophore which both antagonizes an NMDA receptor and augments endogenous GABA inhibition. The agent can be administered orally and, in certain embodiments, after the step of oral administration, the agent can be transported into the nervous system of the subject by an active transport shuttle mechanism. In preferred embodiments, the anti-epileptogenic agent is a β-amino anionic compound, where an anionic moiety is selected from the group consisting of carboxylate, sulfate, sulfonate, sulfinate, sulfamate, tetrazolyl, phosphate, phosphonate, phosphinate, and phosphorothioate. In certain embodiments, the agent is a β-amino acid, but is preferably not β-alanine.

In another aspect, the invention provides a method for inhibiting epileptogenesis in a subject. The method includes administering to a subject in need thereof an effective amount of a compound of the formula:

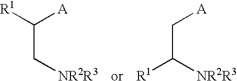

where A is an anionic group at physiological pH; R¹ is alkyl, alkenyl, alkynyl, cycloalkyl, aryl, alkoxy, aryloxy, alkylcarbonyl, arylcarbonyl, alkoxycarbonyl, aryloxycarbonyl, amino, hydroxy, cyano, halogen, carboxyl, alkoxycarbonyloxy, aryloxycarbonyloxy or aminocarbonyl; and R² and R³ are each independently hydrogen, alkyl, alkenyl, alkynyl, cycloalkyl, aryl, alkylcarbonyl, arylcarbonyl, alkoxycarbonyl, or aryloxycrbonyl; or R² and R³, taken together with the nitrogen to which they are attached, form an unsubstituted or substituted heterocycle having from 3 to 7 atoms in the heterocyclic ring; or a pharmaceutically acceptable salt or ester thereof; such that epileptogenesis is inhibited.

In another aspect, the invention provides a method for inhibiting epileptogenesis in a subject. The method includes the step of administering to a subject in need thereof an effective amount of a compound represented by the formula:

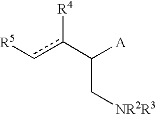

where the dashed line represents an optional single/double bond (of either E- or Z-configuration); A is an anionic group at physiological pH; R² and R³ are each independently hydrogen, alkyl, alkenyl, alkynyl, cycloalkyl, aryl, alkylcarbonyl, arylcarbonyl, alkoxycarbonyl, or aryloxycarbonyl; or R² and R³, taken together with the nitrogen to which they are attached, form an unsubstituted or substituted heterocycle having from 3 to 7 atoms in the heterocyclic ring; R⁴ and R⁵ are each independently hydrogen, alkyl, alkenyl, alkynyl, cycloalkyl, aryl, alkylcarbonyl, arylcarbonyl, alkoxycarbonyl, aryloxycarbonyl, amino, hydroxy, cyano, alkoxy, aryloxy, carboxyl, alkoxycarbonyl, aryloxycarbonyl; or R⁴ and R⁵, taken together, form a substituted or unsubstituted carbocyclic or heterocyclic ring having from 5 to 15 atoms in the ring; or a pharmaceutically acceptable salt or ester thereof; such that epileptogenesis is inhibited.

In another aspect, the invention provides a method for inhibiting a convulsive disorder in a subject. The method includes the step of administering to a subject in need thereof an effective amount of a β-amino anionic compound such that the convulsive disorder is inhibited; provided that the β-amino anionic compound is not β-alanine or taurine.

In another aspect, the invention provides an anti-epileptogenic compound of the formula:

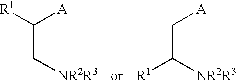

where A is an anionic group at physiological pH; R¹ is alkyl, alkenyl, alkynyl, cycloalkyl, aryl, alkoxy, aryloxy, alkylcarbonyl, arylcarbonyl, alkoxycarbonyl, aryloxycarbonyl, amino, hydroxy, cyano, nitro, thiol, thiolalkyl, halogen, carboxyl, alkoxycarbonyloxy, aryloxycarbonyloxy or aminocarbonyl; and R² and R³ are each independently hydrogen, alkyl, alkenyl, alkynyl, cycloalkyl, aryl, alkylcarbonyl, arylcarbonyl, alkoxycarbonyl, or aryloxycarbonyl; or R² and R³, taken together with the nitrogen to which they are attached, form an unsubstituted or substituted heterocycle having from 3 to 7 atoms in the heterocyclic ring; or a pharmaceutically acceptable salt or ester thereof; wherein the anti-epileptogenic compound has anti-epileptogenic activity. In preferred embodiments, A represents carboxylate.

In certain preferred embodiments, the compound is selected from the group consisting of α-cyclohexyl-β-alanine, α-(4-tert-butylcyclohexyl)-β-alanine, α-(4-phenylcyclohexyl)-β-alanine, α-cyclododecyl-β-alanine, β-(p-methoxyphenethyl)-β-alanine, and β-(p-methylphenethyl)-β-alanine, and pharmaceutically acceptable salts thereof; or the compound is selected from the group consisting of β-(4-trifluoromethylphenyl)-β-alanine and β-[2-(4-hydroxy-3-methoxyphenyl)ethyl]-β-alanine, and pharmaceutically acceptable salts thereof; or the compound is selected from the group consisting of β-(3-pentyl)-β-alanine and β-(4-methylcyclohexyl)-β-alanine, and pharmaceutically acceptable salts thereof.

In still another aspect, the invention provides a dioxapiperazine compound of the formula:

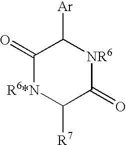

where Ar represents an unsubstituted or substituted aryl group; R⁶ and R⁶* are each independently hydrogen, alkyl, alkylcarbonyl, arylcarbonyl, alkoxycarbonyl or aryloxycarbonyl; and R⁷ is hydrogen, alkyl, mercaptoalkyl, alkenyl, alkynyl, cycloalkyl, aryl, alkylcarbonyl, arylcarbonyl, alkoxycarbonyl, aryloxycarbonyl, cyano, carboxyl, alkoxycarbonyl, aryloxycarbonyl, or —(CH₂)ₙ—Y, where n is an integer from 1 to 4 and Y is hydrogen or a heterocyclic moiety selected from the group consisting of thiazolyl, triazolyl, and imidazolyl; provided that if Ar is an unsubstituted phenyl group, R⁷ is not hydrogen, methyl or phenyl; or a pharmaceutically acceptable salt thereof.

Methods for inhibiting convulsive disorders in a subject are also disclosed. An effective amount of an agent is administered to a subject in need thereof such that epileptogenesis and ictogenesis is inhibited in the subject. The agent blocks sodium or calcium ion channels, or opens potassium or chloride ion channels; and has at least one activity, e.g., NMDA receptor antagonism, augmentation of endogenous GABA inhibition, calcium binding, iron binding, zinc binding, NO synthase inhibition, and antioxidant activity.

In a desired embodiment, the agent antagonizes NMDA receptors by binding to the NMDA receptors, e.g., by binding to the glycine binding site of the NMDA receptors, and/or augments GABA inhibition by decreasing glial GABA uptake.

In another aspect, the invention provides a method for inhibiting a convulsive disorder. The method includes the step of administering to a subject in need thereof an effective amount of a compound represented by the formula:

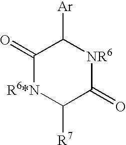

where Ar represents an unsubstituted or substituted aryl group; R⁶ and R⁶* are each independently hydrogen, alkyl, alkylcarbonyl, arylcarbonyl, alkoxycarbonyl or aryloxycarbonyl; and R⁷ is hydrogen, alkyl, mercaptoalkyl, alkenyl, alkynyl, cycloalkyl, aryl, alkylcarbonyl, arylcarbonyl, alkoxycarbonyl, aryloxycarbonyl, cyano, carboxyl, alkoxycarbonyl, aryloxycarbonyl, or —(CH₂)ₙ—Y, n is an integer from 1 to 4 and Y is hydrogen or a heterocyclic moiety, e.g., thiazolyl, triazolyl, and imidazolyl; provided that if Ar is unsubstituted phenyl, R⁷ is not hydrogen, methyl or unsubstituted phenyl; or a pharmaceutically acceptable salt or ester thereof; such that the convulsive disorder is inhibited.

In another aspect, the invention provides a compound of the formula:

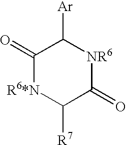

where Ar represents an unsubstituted or substituted aryl group; R⁶ is hydrogen or alkyl, alkylcarbonyl, arylcarbonyl, alkoxycarbonyl or aryloxycarbonyl; R⁶* may be an antioxidant moiety, an NMDA antagonist, an NO synthase inhibitor, an iron chelator moiety, a Ca(II) chelator moiety, or a Zn(II) chelator moiety; and R⁷ is hydrogen, alkyl, mercaptoalkyl, alkenyl, alkynyl, cycloalkyl, aryl, alkylcarbonyl, arylcarbonyl, alkoxycarbonyl, aryloxycarbonyl, cyano, carboxyl, alkoxycarbonyl, aryloxycarbonyl, or —(CH₂)ₙ—Y, where n is an integer from 1 to 4 and Y is a heterocyclic moiety such as thiazolyl, triazolyl, or imidazolyl; or a pharmaceutically acceptable salt thereof. In preferred embodiments, R⁶* is D-α-aminoadipyl and/or R⁷ is mercaptomethyl.

In another aspect, the invention provides a method for concomitantly inhibiting epileptogenesis and ictogenesis, including administration to a subject in need thereof of an effective amount of a compound of the formula:

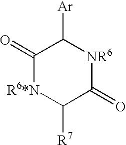

where Ar represents an unsubstituted or substituted aryl group; R⁶ is hydrogen or alkyl, alkylcarbonyl, arylcarbonyl, alkoxycarbonyl or aryloxycarbonyl; R⁶* may be an antioxidant moiety, an NMDA antagonist, an NO synthase inhibitor, an iron chelator moiety, a Ca(II) chelator moiety, or a Zn(II) chelator moiety; and R⁷ is hydrogen, alkyl, mercaptoalkyl, alkenyl, alkynyl, cycloalkyl, aryl, alkylcarbonyl, arylcarbonyl, alkoxycarbonyl, aryloxycarbonyl, cyano, carboxyl, alkoxycarbonyl, aryloxycarbonyl, or —(CH₂)ₙ—Y, where n is an integer from 1 to 4 and Y is a heterocyclic moiety selected from the group consisting of thiazolyl, triazolyl, and imidazolyl; or a pharmaceutically acceptable salt thereof; such that epileptogenesis is inhibited.

In another aspect, the invention provides a method for treating a disorder associated with NMDA receptor antagonism, including the step of administering to a subject in need thereof an effective amount of a compound of the formula:

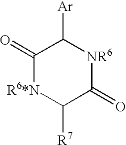

where Ar represents an unsubstituted or substituted aryl group; R⁶ is hydrogen or alkyl, alkylcarbonyl, arylcarbonyl, alkoxycarbonyl or aryloxycarbonyl; R⁶* is an NMDA antagonist moiety; R⁷ is hydrogen, alkyl, mercaptoalkyl, alkenyl, alkynyl, cycloalkyl, aryl, alkylcarbonyl, arylcarbonyl, alkoxycarbonyl, aryloxycarbonyl, cyano, carboxyl, alkoxycarbonyl, aryloxycarbonyl, or —(CH₂)ₙ—Y, where n is an integer from 1 to 4 and Y is a heterocyclic moiety selected from the group consisting of thiazolyl, triazolyl, and imidazolyl; or a pharmaceutically acceptable salt thereof; such that the disorder associated with NMDA receptor antagonism is treated.

In another aspect, the invention provides a method for preparing a β-amino carboxyl compound represented by the formula:

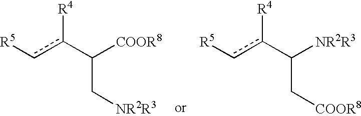

where the dashed line represents an optional single/double bond (of either E- or Z-configuration); R² and R³ are each independently hydrogen, alkyl, alkenyl, alkynyl, cycloalkyl, aryl, alkylcarbonyl, arylcarbonyl, alkoxycarbonyl, or aryloxycarbonyl; or R² and R³, taken together with the nitrogen to which they are attached, form an unsubstituted or substituted heterocycle having from 3 to 7 atoms in the heterocyclic ring; R⁴ and R⁵ are each independently hydrogen, alkyl, alkenyl, alkynyl, cycloalkyl, aryl, alkylcarbonyl, arylcarbonyl, alkoxycarbonyl, aryloxycarbonyl, amino, hydroxy, cyano, carboxyl, alkoxycarbonyl, or aryloxycarbonyl; or R⁴ and R⁵, taken together form a substituted or unsubstituted carbocyclic or heterocyclic ring having from 5 to 15 atoms in the ring; and R⁸ is hydrogen, alkyl, aryl, or an organic or inorganic salt-forming cation. The method includes the step of reacting a compound of the formula:

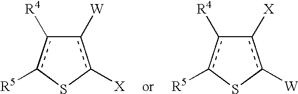

where the dashed lines each represent an optional single bond; X is nitro, azido, or NR²R³, wherein R² and R³ are defined above; W is —CN or —COOR⁸; R⁴ and R⁵ are as defined above; and R⁸ is hydrogen, alkyl, aryl, or an organic or inorganic salt-forming cation; under reductive desulfurization conditions such that the β-amino carboxyl compound is formed.

In another aspect, the invention provides a method for preparing a β-amino carboxyl compound represented by the formula:

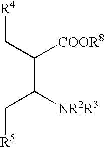

where R² and R³ are each independently hydrogen, alkyl, alkenyl, alkynyl, cycloalkyl, aryl, alkylcarbonyl, arylcarbonyl, alkoxycarbonyl, or aryloxycarbonyl; or R² and R³, taken together with the nitrogen to which they are attached, form an unsubstituted or substituted heterocycle having from 3 to 7 atoms in the heterocyclic ring; R⁴ and R⁵ are each independently hydrogen, alkyl, alkenyl, alkynyl, cycloalkyl, aryl, alkylcarbonyl, arylcarbonyl, alkoxycarbonyl, aryloxycarbonyl, amino, hydroxy, cyano, alkoxy, aryloxy, carboxyl, alkoxycarbonyl, aryloxycarbonyl; or R⁴ and R⁵, taken together, form a substituted or unsubstituted carbocyclic or heterocyclic ring having from 5 to 15 atoms in the ring; and R⁸ is hydrogen, alkyl, aryl, or an organic or inorganic salt-forming cation. The method includes reacting a compound of the formula:

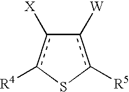

where the dashed lines each represent an optional single/double bond; X is nitro, azido, or NR²R³, R² and R³ are as defined above; W is —CN or —COOR⁸; R⁸ is hydrogen, alkyl, aryl, or an organic or inorganic salt-forming cation; and R⁴ and R⁵ are as defined above; under reductive desulfurization conditions such that the β-amino carboxyl compound of the above formula is formed; provided that if W is —CN, the method comprises the further step of acidification.

The invention also provides a method for inhibiting epileptogenesis and ictogenesis in a subject including administering to a subject in need thereof an effective amount of an agent represented by the formula A-B, where A is a domain having sodium or calcium ion channel blocking activity, or A has potassium or chloride channel opening activity; and B is a domain having has at least one activity, e.g., NMDA receptor antagonism; augmentation of endogenous GABA inhibition, calcium binding, iron binding, zinc binding, NO synthase inhibition, and antioxidant activity, such that epileptogenesis is inhibited in the subject. In preferred embodiments, the domains A and B of the agent are covalently linked. In a preferred embodiment, A is a dioxapiperazine moiety.

In yet another aspect, the invention provides a method for inhibiting epileptogenesis including administering to a subject in need thereof an effective amount of a compound represented by the formula:

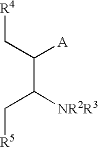

where A is an anionic group at physiological pH; R² and R³ are each independently hydrogen, alkyl, alkenyl, alkynyl, cycloalkyl, aryl, alkylcarbonyl, arylcarbonyl, alkoxycarbonyl, or aryloxycarbonyl; or R² and R³, taken together with the nitrogen to which they are attached, form an unsubstituted or substituted heterocycle having from 3 to 7 atoms in the heterocyclic ring; R⁴ and R⁵ are each independently hydrogen, alkyl, alkenyl, alkynyl, cycloalkyl, aryl, alkylcarbonyl, arylcarbonyl, alkoxycarbonyl, aryloxycarbonyl, amino, hydroxy, cyano, alkoxy, aryloxy, carboxyl, alkoxycarbonyl, or aryloxycarbonyl; or R⁴ and R⁵, taken together, form a substituted or unsubstituted carbocyclic or heterocyclic ring having from 5 to 15 atoms in the ring; or a pharmaceutically acceptable salt or ester thereof; such that epileptogenesis is inhibited.

A method for inhibiting a neurological condition in a subject includes the step of administering to a subject in need thereof an effective amount of an agent which antagonizes an NMDA receptor and augments endogenous GABA inhibition, such that the neurological condition is inhibited in the subject. The neurological condition may be, e.g., stroke, Alzheimer's disease, cancer, and neurodegenerative disease.

Methods for preparing a β-aryl-β-alanine compound are presented, which include reacting an aryl aldehyde with a malonate compound and an ammonium compound under conditions such that a β-aryl-β-alanine compound is formed.

Other methods for inhibiting epileptogenesis include administering to a subject in need thereof an effective amount of a compound represented by the formula:

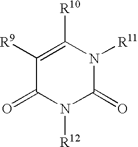

where R⁹ and R¹⁰ may each independently be hydrogen, alkyl, alkenyl, alkynyl, aryl, alkoxy, aryloxy, alkylcarbonyl, arylcarbonyl, alkoxycarbonyl, aryloxycarbonyl, amino, hydroxy, thiol, alkylthiol, nitro, cyano, halogen, carboxyl, alkoxycarbonyloxy, aryloxycarbonyloxy and aminocarbonyl; or R⁹ and R¹⁰, together with the two-carbon unit to which they are attached, are joined to form a carbocyclic or heterocyclic ring having from 4 to 8 members in the ring; and R ¹¹is hydrogen, alkyl, alkenyl, alkynyl, cycloalkyl, aryl, alkylcarbonyl, arylcarbonyl, alkoxycarbonyl, or aryloxycarbonyl; or R¹⁰ and R¹¹, together with the carbon atom and nitrogen atom to which they are respectively attached, are joined to form a heterocyclic ring having from 4 to 8 members in the ring; and R¹² is selected from the group consisting of hydrogen, alkyl, aryl and a carbohydrate; or a pharmaceutically acceptable salt or ester thereof; such that epileptogenesis is inhibited.

In another aspect, a method for inhibiting epileptogenesis includes administering to a subject in need thereof an effective amount of a compound represented by the formula:

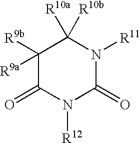

where R^{9a}, R^{9b}, R^{10a}, R^{10b} may each independently be hydrogen, alkyl, alkenyl, alkynyl, aryl, alkoxy, aryloxy, alkylcarbonyl, arylcarbonyl, alkoxycarbonyl, aryloxycarbonyl, amino, hydroxy, thiol, alkylthiol, nitro, cyano, halogen, carboxyl, alkoxycarbonyloxy, aryloxycarbonyloxy and aminocarbonyl; or R^{9a} and R^{9b}, together with the two-carbon unit to which they are attached, are joined to form a carbocyclic or heterocyclic ring having from 4 to 8 members in the ring; or R^{10a} and R^{10b}, together with the two-carbon unit to which they are attached, are joined to form a carbocyclic or heterocyclic ring having from 4 to 8 members in the ring; or one of R^{9a} and R^{9b} is joined with one of R^{10a} and R^{10b}, together with the two-carbon unit to which they are attached, to form a carbocyclic or heterocyclic ring having from 4 to 8 members in the ring; R¹¹ is hydrogen, alkyl, alkenyl, alkynyl, cycloalkyl, aryl, alkylcarbonyl, arylcarbonyl, alkoxycarbonyl, or aryloxycarbonyl; or one of R^{10b} and R^{10b} is joined with R¹¹, together with the carbon atom and nitrogen atom to which they are respectively attached, to form a heterocyclic ring having from 4 to 8 members in the ring; and R¹² is selected from the group consisting of hydrogen, alkyl, aryl and a carbohydrate (such as a sugar, e.g., ribose or deoxyribose); or a pharmaceutically acceptable salt or ester thereof; such that epileptogenesis is inhibited.

Pharmacophore modeling methods for identifying compounds which can prevent and/or inhibit epileptogenesis in a subject are part of the invention and feature the examination of the structures of two or more compounds which are known to cause a direct or indirect pharmacological effect on a protein or a molecule which is involved in epileptogenesis. These proteins and molecules which are involved in epileptogenesis include cell-surface receptor molecules (e.g., an NMDA receptor) or a molecule that is involved in transport of neurotransmitters (e.g., a GABA transporter). Preferably, the structures of these compounds each include one or more pharmacophores which can exert at least some of the pharmacological effect of the compound. The methods of the invention also include determining average pharmacophore structure(s) (e.g., carbon backbone structures and/or a three-dimensional space filling structures) based on the pharmacophore structures of the two or more compounds. New compounds having one or more of the average pharmacophore structures can be chosen using these methods such as shown in Example 1.

In related embodiments, these methods feature the examination of the structures of two or more compounds which are known to cause a direct or indirect pharmacological effect on two or more proteins or molecules which are involved in epileptogenesis. The new compound which is chosen will preferably have one or more pharmacophores which are active on different proteins or molecules involved with epileptogenesis.

In a preferred embodiment, a new compound which is chosen (e.g., designed) by these methods of the invention inhibits epileptogenesis in a subject. It is a further object of the invention to provide compounds and methods for treatment of stroke, Alzheimer's disease and neurodegenerative disorders. It is a further object of the invention to provide novel anticonvulsant agents. It is a further object of the invention to provide compounds and methods for treating stroke and pain. These and other objects, features, and advantages of the invention will be apparent from the following description and claims.

### BRIEF DESCRIPTION OF THE DRAWINGS

FIG. 1 depicts exemplary pyrimidine and dihydropyrimidine compounds useful in the methods of the invention.

FIG. 2 depicts exemplary synthetic schemes for preparing pyrimidine and dihydropyrimidine compounds of the invention.

FIG. 3 depicts one embodiment of a synthesis of β-amino acids of the invention.

FIG. 4 is a flow chart showing a scheme for purification of β-amino acids.

### DETAILED DESCRIPTION OF THE INVENTION

This invention pertains to methods and agents useful for the treatment of epilepsy and convulsive disorders, for inhibition of epileptogenesis, and for inhibition of ictogenesis; and to methods for preparing anti-convulsive and anti-epileptogenic agents of the invention. The invention further pertains to pharmaceutical compositions for treatment of convulsive disorders, and to kits including the anti-convulsive compounds of the invention.

### Definitions

For convenience, certain terms used in the specification, examples, and appended claims are collected here.

The language “a process in a pathway associated with epileptogenesis” includes biochemical processes or events which take place during Phase 1 or Phase 2 epileptogenesis and lead to epileptogenic changes in tissue, i.e., in tissues of the central nervous system (CNS), e.g., the brain. Examples of processes in pathways associated with epileptogenesis are discussed in more detail, infra.

The language “a disorder associated with NMDA receptor antagonism,” includes disorders of a subject where abnormal (e.g., excessive) activity of NMDA receptors can be treated by antagonism of an NMDA receptor. Epilepsy is a disorder associated with excessive NMDA-mediated activity. Other non-limiting examples of disorders associated with excessive NMDA-mediated activity include pain, stroke, anxiety, schizophrenia, other psychoses, cerebral ischemia, Huntington's chorea, motor neuron disease, Alzheimer's disease, AIDS dementia and other disorders (in humans or animals) where excessive activity of NMDA receptors is a cause, at least in part, of the disorder. See, e.g., Schoepp et al., Eur. J. Pharmacol. 203:237-243 (1991); Leeson et al., J. Med. Chem. 34:1243-1252 (1991); Kulagowski et al., J. Med. Chem. 37:1402-1405 (1994); Mallamo et al., J. Med. Chem. 37:4438-4448 (1994); and references cited therein.

The term “convulsive disorder” includes disorders where the subject suffers from convulsions, e.g., convulsions due to epileptic seizure. Convulsive disorders include, but are not limited to, epilepsy and non-epileptic convulsions, e.g., convulsions due to administration of a convulsive agent to the subject.

The term “inhibition of epileptogenesis” includes preventing, slowing, halting, or reversing the process of epileptogenesis.

The term “anti-epileptogenic agent” includes agents which are capable of inhibiting epileptogenesis when the agent is administered to a subject.

The term “anticonvulsant agent” includes agents capable of inhibiting (e.g., preventing, slowing, halting, or reversing) ictogenesis when the agent is administered to a subject.

The term “pharmacophore” is known in the art, and includes molecular moieties capable of exerting a selected biochemical effect, e.g., inhibition of an enzyme, binding to a receptor, chelation of an ion, and the like. A selected pharmacophore can have more than one biochemical effect, e.g., can be an inhibitor of one enzyme and an agonist of a second enzyme. A therapeutic agent can include one or more pharmacophores, which can have the same or different biochemical activities. The skilled practitioner will recognize that a number of pharmacophores with similar structures and/or properties (e.g., biological effects) may be combined to predict or design an optimized or “average pharmacophore” structure. Such an average pharmacophore structure may provide a more desired level of biological effect that the individual pharmacophores used to create the average structure.

An “anionic group” refers to a group that is negatively charged at physiological pH. Preferred anionic groups include carboxylate, sulfate, sulfonate, sulfinate, sulfamate, tetrazolyl, phosphate, phosphonate, phosphinate, or phosphorothioate or functional equivalents thereof. “Functional equivalents” of anionic groups include bioisosteres, e.g., bioisosteres of a carboxylate group. Bioisosteres encompass both classical bioisosteric equivalents and non-classical bioisosteric equivalents. Classical and non-classical bioisosteres are known in the art. See, e.g., Silverman, R. B. The Organic Chemistry of Drug Design and Drug Action, Academic Press, Inc.: San Diego, Calif., 1992, pp. 19-23. A particularly preferred anionic group is a carboxylate.

The term “β-amino anionic compound” includes compounds having an amino group, such as —NR^{a}R^{b} (where R^{a} and R^{b} may each independently be hydrogen, alkyl, alkenyl, alkynyl, cycloalkyl, aryl, alkylcarbonyl, arylcarbonyl, alkoxycarbonyl, or aryloxycarbonyl, or R^{a} and R^{b}, taken together with the nitrogen atom to which they are attached, form a cyclic moiety having from 3 to 8 atoms in the ring) separated from an anionic group by a two-carbon spacer unit. Thus, for example, a β-amino anionic compound can be represented by the substructural formula A-C—C—NR^{a}R^{b}, where A is an anionic group. Preferred β-amino anionic compounds include β-amino acids and analogs thereof. In certain preferred embodiments, the β-amino anionic compound is not β-alanine or taurine.

The language “reductive desulfurization” is known in the art, and refers to the process of reductively eliminating sulfur from a compound. Conditions for reductive desulfurization are known in the art and include, e.g., treatment with TiCl₄/LiAlH₄ or Raney nickel/H₂. See generally, Kharash, N. and Meyers, C. Y., “The Chemistry of Organic Sulfur Compounds,” Pergamon Press, New York (1966), Vol. 2.

The term “subject” is known in the art, and refers to a warm-blooded animal, more preferably a mammal, including non-human animals such as rats, mice, cats, dogs, sheep, horses, cattle, in addition to apes, monkeys, and humans. In a preferred embodiment, the subject is a human.

Unless specifically indicated, the chemical groups of the present invention may be substituted or unsubstituted. Further, unless specifically indicated, the chemical substituents may in turn be substituted or unsubstituted. In addition, multiple substituents may be present on a chemical group or substituent. Examples of substituents include alkenyl, alkynyl, halogen, hydroxyl, alkylcarbonyloxy, arylcarbonyloxy, alkoxycarbonyloxy, aryloxycarbonyloxy, carboxyl, alkylcarbonyl, arylcarbonyl, alkoxycarbonyl, aminocarbonyl, alkylaminocarbonyl, dialkylaminocarbonyl, alkylthiocarbonyl, alkoxyl, formyl, trimethylsilyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amido, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, and aromatic or heteroaromatic moieties

The term “alkyl” refers to saturated aliphatic groups, including straight-chain alkyl groups, branched-chain alkyl groups, cycloalkyl, heterocyclyl, cycloalkyl (alicyclic) groups, alkyl substituted cycloalkyl groups, and cycloalkyl substituted alkyl groups. In preferred embodiments, a straight chain or branched chain alkyl has 30 or fewer carbon atoms in its backbone (e.g., C₁-C₃₀ for straight chain, C₃-C₃₀ for branched chain), and more preferably has 20 or fewer carbon atoms in the backbone. Likewise, preferred cycloalkyls have from 4-10 carbon atoms in their ring structure, and more preferably have 5, 6 or 7 carbons in the ring structure.

Moreover, alkyl (e.g., methyl, ethyl, propyl, butyl, pentyl, hexyl, etc.) includes both “unsubstituted alkyl” and “substituted alkyl,” the latter of which refers to alkyl moieties having substituents replacing a hydrogen on one or more carbons of the hydrocarbon backbone. Such substituents can include, for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, or an aromatic or heteroaromatic moiety. It will be understood by those skilled in the art that the moieties substituted on the hydrocarbon chain can themselves be substituted, if appropriate. Cycloalkyls can be further substituted, e.g., with the substituents described above. An “aralkyl” moiety is an alkyl substituted with an aryl (e.g., phenylmethyl (i.e., benzyl)).

The term “aryl” includes 5- and 6-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Aryl groups also include polycyclic fused aromatic groups such as naphthyl, quinolyl, indolyl, and the like. Those aryl groups having heteroatoms in the ring structure may also be referred to as “aryl heterocycles,” “heteroaryls” or “heteroaromatics.” The aromatic ring (e.g., phenyl, indole, thiophene) can be substituted at one or more ring positions with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, or an aromatic or heteroaromatic moiety. Aryl groups can also be fused or bridged with alicyclic or heterocyclic rings which are not aromatic so as to form a polycycle such as tetralin.

The terms “alkenyl” and “alkynyl” include unsaturated aliphatic groups analogous in length and possible substitution to the alkyls described above, but that contain at least one double or triple bond respectively and at least two adjacent carbon atoms.

As used in the description and drawings herein, an “optional single/double bond” is represented by a solid line together with a dashed line, and refers to a covalent linkage between two carbon atoms which can be either a single bond or a double bond of either E- or Z-configuration where appropriate. For example, the structure:

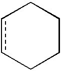

can represent either cyclohexane or cyclohexene.

Unless the number of carbons is otherwise specified, “lower alkyl” means an alkyl group as defined above, but having from one to ten carbons, more preferably from one to six carbon atoms in its backbone structure. Likewise, “lower alkenyl” and “lower alkynyl” have similar chain lengths. Preferred alkyl groups are lower alkyls.

The terms “heterocyclyl” or “heterocyclic group” refer to 3- to 10-membered ring structures, more preferably 4- to 7-membered rings, which ring structures include one or more heteroatoms, e.g, two, three, or four. Heterocyclyl groups include pyrrolidine, oxolane, thiolane, piperidine, piperazine, morpholine, lactones, lactams such as azetidinones and pyrrolidinones, sultams, sultones, and the like. The heterocyclic ring can be substituted at one or more positions with such substituents as described above, including halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, or an aromatic or heteroaromatic moiety.

The terms “polycyclyl” or “polycyclic group” refer to two or more cyclic rings (e.g., cycloalkyls, cycloalkenyls, cycloalkynyls, aryls and/or heterocyclyls) where two or more carbons are common to two adjoining rings, e.g., the rings are “fused rings.” Rings that are joined through non-adjacent atoms are termed “bridged” rings. Each of the rings of the polycycle can be substituted with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, or an aromatic or heteroaromatic moiety.

The term “heteroatom” as used herein means an atom of any element other than carbon or hydrogen. Preferred heteroatoms are nitrogen, oxygen, sulfur and phosphorus.

The term “aryl aldehyde,” as used herein, refers to a compound represented by the formula Ar—C(O)H, where Ar is an aryl moiety (as described above) and —C(O)H is a formyl or aldehydo group. In a preferred embodiment, the aryl aldehyde is a (substituted or unsubstituted) benzaldehyde. A variety of aryl aldehydes are commercially available, or can be prepared by routine procedures from commercially available precursors. Procedures for the preparation of aryl aldehydes include the Vilsmeier-Haack reaction (see, e.g., Jutz, Adv. Org. Chem. 9, pt. 1, 225-342 (1976)), the Gatterman reaction (Truce, Org. React. 9, 37-72 (1957)), the Gatterman-Koch reaction (Crounse, Org. React. 5, 290-300 (1949)), and the Reimer-Tiemann reaction (Wynberg and Meijer, Org. React. 28, 1-36 (1982)).

It will be noted that the structure of some of the compounds of this invention includes asymmetric carbon atoms. It is to be understood accordingly that the isomers arising from such asymmetry (e.g., all enantiomers and diastereomers) are included within the scope of this invention unless indicated otherwise. That is, unless otherwise stipulated, any chiral carbon center may be of either (R)- or (S)-stereochemistry. Such isomers can be obtained in substantially pure form by classical separation techniques and by stereochemically controlled synthesis. Furthermore, alkenes can include either the E- or Z-geometry, where appropriate.

I. Methods for Treating Convulsive Disorders

In one aspect, the invention provides methods for treating convulsive disorders, including epilepsy.

In one aspect, the invention provides a method for inhibiting epileptogenesis in a subject. The method includes administering to a subject in need thereof an effective amount of an agent which modulates a process in a pathway associated with epileptogenesis such that epileptogenesis is inhibited in the subject.

As noted above, upregulation of excitatory coupling between neurons, mediated by N-methyl-D-aspartate (NMDA) receptors, and downregulation of inhibitory coupling between neurons, mediated by gamma-amino-butyric acid (GABA) receptors, have both been implicated in epileptogenesis. Other processes in pathways associated with epileptogenesis include release of nitric oxide (NO), a neurotransmitter implicated in epileptogenesis; release of calcium (Ca²⁺), which may mediate damage to neurons when released in excess; neurotoxicity due to excess zinc (Zn²⁺); neurotoxicity due to excess iron (Fe²⁺); and neurotoxicity due to oxidative cell damage. Accordingly, in preferred embodiments, an agent to be administered to a subject to inhibit epileptogenesis preferably is capable of inhibiting one or more processes in at least one pathway associated with epileptogenesis. For example, an agent useful for inhibition of epileptogenesis can reduce the release of, or attenuate the epileptogenic effect of, NO in brain tissue; antagonize an NMDA receptor; augment endogenous GABA inhibition; block voltage-gated ion channels; reduce the release of, reduce the free concentration of (e.g., by chelation), or otherwise reduce the epileptogenic effect of cations including Ca²⁺, Zn²⁺, or Fe²⁺; inhibit oxidative cell damage; or the like. In certain preferred embodiments, an agent to be administered to a subject to inhibit epileptogenesis is capable of inhibiting at least two processes in at least one pathway associated with epileptogenesis.

Non-limiting examples of pharmacophores which can modulate a process in a pathway associated with epileptogenesis include:

inhibitors of NO synthase such as L-arginine and alkylated derivatives thereof,

antagonists of NMDA receptors such as (R)-α-amino acids. See, e.g., Leeson, P. D. and Iverson, L. L., J. Med. Chem. (1994) 37:4053-4067 for a general review of inhibitors of the NMDA receptor;

augmenters of endogenous GABA inhibition such as inactivators of GABA aminotransferase like gamma-vinyl-GABA. See, e.g., Krogsgaard-Larsen, P., et al., J. Med. Chem. (1994) 37:2489-2505) for a review of GABA receptor agonists and antagonists;

a chelators of Ca²⁺, Zn²⁺, or Fe²⁺ such as EDTA, EGTA, TNTA, 2,2-bipyridine-4,4,-dicarboxylate, enterobactin, porphyrins, crown ethers, azacrown ethers; and

antioxidants such as vitamins C and E, carotenoids such as β-carotene, butylated phenols, Trolox (a tocopherol analog), selenium, and glutathione.

In one preferred embodiment, the agent antagonizes an NMDA receptor and augments endogenous GABA inhibition. In certain preferred embodiments, the agent is administered orally. Preferably, after oral administration, the agent is transported to the nervous system of the subject by an active transport shuttle mechanism. A non-limiting example of an active transport shuttle is the large neutral amino acid transporter, which is capable of transporting amino acids across the blood-brain barrier (BBB).

In another embodiment, the invention provides a method for inhibiting epileptogenesis. The method includes the step of administering to a subject in need thereof an effective amount of a compound of the formula (Formula I):

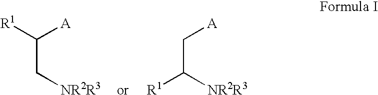

where A is an anionic group at physiological pH; R¹ is alkyl, alkenyl, alkynyl, cycloalkyl, aryl, alkoxy, aryloxy, alkylcarbonyl, arylcarbonyl, alkoxycarbonyl, aryloxycarbonyl, amino, hydroxy, cyano, halogen, carboxyl, alkoxycarbonyloxy, aryloxycarbonyloxy or aminocarbonyl; and R² and R³ are each independently hydrogen, alkyl, alkenyl, alkynyl, cycloalkyl, aryl, alkylcarbonyl, arylcarbonyl, alkoxycarbonyl, or aryloxycarbonyl; or R² and R³, taken together with the nitrogen to which they are attached, form an unsubstituted or substituted heterocycle having from 3 to 7 atoms in the heterocyclic ring; or a pharmaceutically acceptable salt or ester thereof; such that epileptogenesis is inhibited. In a preferred embodiment, R² and R³ are both hydrogen.

In certain embodiments, the compound of Formula I can be represented by the formula (Formula II):

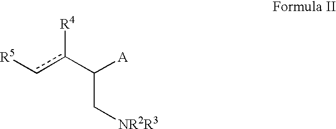

where the dashed line represents an optional single bond; R⁴ and R⁵ are each independently hydrogen, alkyl, alkenyl, alkynyl, cycloalkyl, aryl, alkylcarbonyl, arylcarbonyl, alkoxycarbonyl, aryloxycarbonyl, amino, hydroxy, cyano, alkoxy, aryloxy, carboxyl, alkoxycarbonyl, aryloxycarbonyl, heterocyclic; or R⁴ and R⁵, taken together, form a substituted or unsubstituted carbocyclic or heterocyclic ring having from 5 to 15 atoms (more preferably 5 to 8 atoms) in the ring; and A, R² and R³ are as defined above; or a pharmaceutically acceptable salt or ester thereof, such that epileptogenesis is inhibited.

In another embodiment, the invention provides a method for inhibiting epileptogenesis. The method includes the step of administering to a subject in need thereof an effective amount of a compound represented by the formula (Formula III):

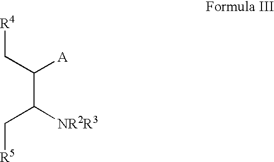

where A, R², R³, R⁴, and R⁵ are as defined above; or a pharmaceutically acceptable salt or ester thereof; such that epileptogenesis is inhibited. In a preferred embodiment, A is a carboxylate. In a particularly preferred embodiment, A is carboxylate, R⁴ is hydrogen, and R⁵ is a (substituted or unsubstituted) aryl group. In another preferred embodiment, R⁴ and R⁵ taken together, form a 6-membered ring as in, e.g., 2-, 3-, or 4-aminobenzoic acid, particularly anthralinic acid.

In another embodiment, the invention provides a method for inhibiting epileptogenesis. The method includes the step of administering to a subject in need thereof an effective amount of a compound selected from the group consisting of α,α-disubstituted β-alanines, α,β-disubstituted β-alanines, β,β-disubstituted β-alanines, α,β,α-trisubstituted β-alanines, α,β,β-trisubstituted β-alanines, α,α,N-trisubstituted β-alanines, α,β,N-trisubstituted β-alanines, β,β,N-trisubstituted β-alanines, α,α,N,N-tetrasubstituted β-alanines, α,β,N,N-tetrasubstituted β-alanines, β,β,N,N-tetrasubstituted β-alanines, α,α,β,β-tetrasubstituted β-alanines, α,α,β,N-tetrasubstituted β-alanines, α,β,β,N-tetrasubstituted β-alanines, α,α,β,N,N-pentasubstituted β-alanines, α,β,β,N,N-pentasubstituted β-alanines, α,α,β,β,N-pentasubstituted β-alanines, α,α,β,β,N,N-hexasubstituted β-alanines including all stereoisomers; or pharmaceutically acceptable salts or esters thereof, such that epileptogenesis is inhibited.

The step of administering to the subject can include administering to the subject a compound which is metabolized to an anti-convulsant and/or anti-epileptogenic compound of the invention. For example, the methods of the invention include the use of prodrugs which are converted in vivo to the therapeutic compounds of the invention. See, e.g, Silverman, ch. 8, cited above. Such prodrugs can be used to alter the biodistribution to allow compounds which would not typically cross the blood-brain barrier to cross the blood-brain barrier, or the pharmacokinetics of the therapeutic compound. For example, an anionic group, e.g., a carboxylate group, can be esterified with an ethyl or a fatty group to yield a carboxylic ester. When the carboxylic ester is administered to a subject, the ester can be cleaved, enzymatically or non-enzymatically, to reveal the anionic group.

In another illustrative embodiment, the methods of the invention include administering to the subject a derivative of uracil or an analog thereof (including substituted pyrimidines, UMP and uridine, or analogs thereof). Administration of a uracil compound or metabolite thereof, such as a dihydrouracil or a β-ureidopropionate, can result in the in vivo formation of an active compound of the invention. Accordingly, in a preferred embodiment, the methods of the invention may include the step of administering to a subject in need thereof an effective amount of a substituted or unsubstituted uracil, dihydrouracil or β-ureidopropionate compound, or a derivative or analog thereof (or a pharmaceutically acceptable salt or ester thereof), in an amount effective to treat a convulsive disorder and/or to inhibit epileptogenesis, e.g., by in vivo conversion of the uracil, dihydrouracil or β-ureidopropionate compound to a β-amino acid compound effective to treat or prevent the convulsive disorder.

Thus, in certain embodiments, preferred compounds for administration to a subject include pyrimidines such as substituted uracils which can be converted in vivo to β-amino anionic compounds. In a preferred embodiment, the compound can be represented by the formula (Formula V):

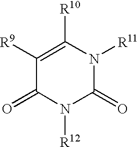

### Formula V

where R⁹ and R¹⁰ may each independently be hydrogen, alkyl (including cycloalkyl, heterocyclyl, and aralkyl), alkenyl, alkynyl, aryl, alkoxy, aryloxy, alkylcarbonyl, arylcarbonyl, alkoxycarbonyl, aryloxycarbonyl, amino (including unsubstituted and substituted amino), hydroxy, thiol, alkylthiol, nitro, cyano, halogen, carboxyl, alkoxycarbonyloxy, aryloxycarbonyloxy or aminocarbonyl; or R⁹ and R¹⁰, together with the two-carbon unit to which they are attached, are joined to form a carbocyclic or heterocyclic ring having from 4 to 8 members in the ring; and R¹¹ is hydrogen, alkyl, alkenyl, alkynyl, cycloalkyl, aryl, alkylcarbonyl, arylcarbonyl, alkoxycarbonyl, or aryloxycarbonyl; or R¹⁰ and R¹¹, together with the carbon atom and nitrogen atom to which they are respectively attached, are joined to form a heterocyclic ring having from 4 to 8 members in the ring; and R¹² is selected from the group consisting of hydrogen, alkyl, aryl and a carbohydrate (such as a sugar, e.g., ribose or deoxyribose); or a pharmaceutically acceptable salt or ester thereof. In another embodiment, the compound can be represented by the formula (Formula Va):

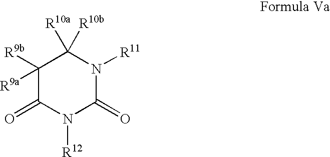

where R^{9a}, R^{9b}, R^{10a}, R^{10b} may each independently be hydrogen, alkyl (including cycloalkyl, heterocyclyl, and aralkyl), alkenyl, alkynyl, aryl, alkoxy, aryloxy, alkylcarbonyl, arylcarbonyl, alkoxycarbonyl, aryloxycarbonyl, amino (including unsubstituted and substituted amino), hydroxy, thiol, alkylthiol, nitro, cyano, halogen, carboxyl, alkoxycarbonyloxy, aryloxycarbonyloxy or aminocarbonyl; or R^{9a} and R^{9b}, together with the two-carbon unit to which they are attached, are joined to form a carbocyclic or heterocyclic ring having from 4 to 8 members in the ring; or R^{10a} and R^{10b}, together with the two-carbon unit to which they are attached, are joined to form a carbocyclic or heterocyclic ring having from 4 to 8 members in the ring; or one of R^{9a} and R^{9b} is joined with one of R^{10a} and R^{10b}, together with the two-carbon unit to which they are attached, to form a carbocyclic or heterocyclic ring having from 4 to 8 members in the ring; R¹¹ is hydrogen, alkyl, alkenyl, alkynyl, cycloalkyl, aryl, alkylcarbonyl, arylcarbonyl, alkoxycarbonyl, or aryloxycarbonyl; or one of R^{10b} and R^{10b} is joined with R¹¹, together with the carbon atom and nitrogen atom to which they are respectively attached, to form a heterocyclic ring having from 4 to 8 members in the ring; and R¹² is selected from the group consisting of hydrogen, alkyl, aryl and a carbohydrate (such as a sugar, e.g., ribose or deoxyribose); or a pharmaceutically acceptable salt or ester thereof.

Pyrimidine compounds, such as 5-fluorouracil (5FU), have been used as anti-neoplastic agents. The anti-cancer activity of 5FU and similar compounds is believed to be due to a “suicide substrate” mechanism where the 5FU inhibits thymidylate synthase, an enzyme important in DNA synthesis. In preferred embodiments, pyrimidine and dihydropyrimidine compounds administered according to the invention for the treatment of convulsive disorders (inhibition of epileptogenesis) do not significantly inhibit thymidylate synthase. Without wishing to be bound by theory, it is believed that inhibition of thymidylate synthase by pyrimidine compounds is increased by the presence of electronegative groups at the 5-position of the pyrimidine ring (i.e., R⁹ of Formula Va), and can therefore be decreased by providing such compounds with non-electronegative groups at the 5-position of the pyrimidine ring (i.e., R⁹ of Formula Va). It is further believed that by providing substituents with sufficient steric bulk to decrease the ability of the pyrimidine compound to bind to thymidylate synthase, inhibition of thymidylate synthase can be decreased. Thus, in preferred embodiments, in a compound of Formula V for administration according to the present invention, R⁹ is a non-electronegative (i.e., neutral or electropositive) group (e.g., alkyl, aryl, or the like). In preferred embodiments, at least one of R⁹ and R¹⁰ of Formula V is a sterically bulky group (e.g., long-chain or branched alkyl, substituted aryl, or the like), or R⁹ and R¹⁰ are joined to form a carbocyclic or heterocyclic ring.

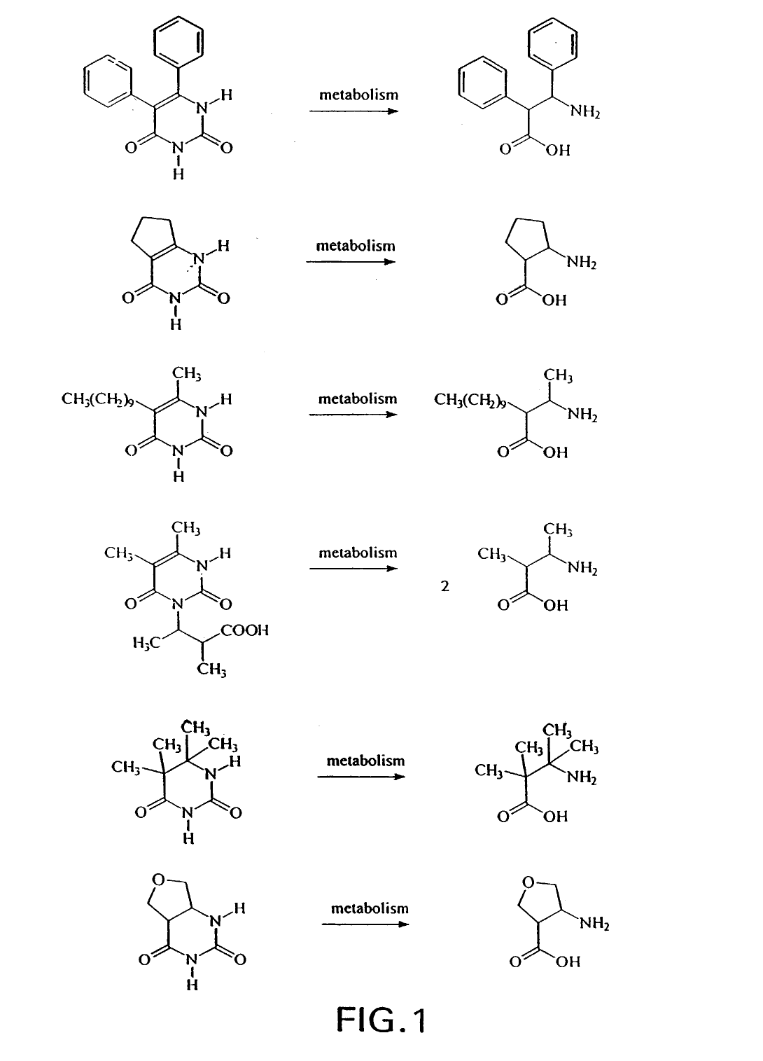

Non-limiting examples of pyrimidine and dihydropyrimidine compounds for use according to the invention, together with illustrative active metabolites thereof, are shown in FIG. 1.

The use of substituted or unsubstituted uracils, and derivatives or analogs thereof, may be especially advantageous as certain uracil compounds have been found to have anti-ictogenic properties (only) when tested in an anti-seizure model in rats. See, e.g., Medicinal Chemistry Volume V; W. J. Close, L. Doub, M. A. Spielman; Editor W. H. Hartung; John Wiley and Sons 1961). Thus, the prodrug form of the compound (a uracil) can have anti-seizure activity, while the metabolically-produced β-amino anionic compounds can have anti-epileptogenic and/or anti-convulsive activity. These activities, individually and in combination, can provide effective therapy for convulsive disorders in mammals (including humans).

In certain embodiments, an active agent of the invention antagonizes NMDA receptors by binding to the glycine binding site of the NMDA receptors. In certain preferred embodiments, the agent augments GABA inhibition by decreasing glial GABA uptake. In certain other embodiments, the agent is administered orally. In yet other embodiments, the method further includes administering the agent in a pharmaceutically acceptable vehicle.

In still another embodiment, the invention provides a method of inhibiting a convulsive disorder. The method includes the step of administering to a subject in need thereof an effective amount of a β-amino anionic compound such that the convulsive disorder is inhibited; provided that the β-amino anionic compound is not β-alanine or taurine.

In another embodiment, the invention provides a method for inhibiting both a convulsive disorder and epileptogenesis in a subject. The method includes the step of administering to a subject in need thereof an effective amount of an agent which blocks sodium or calcium ion channels, or opens potassium or chloride ion channels; and has at least one activity selected from the group consisting of NMDA receptor antagonism, augmentation of endogenous GABA inhibition, calcium binding, iron binding, zinc binding, NO synthase inhibition, and antioxidant activity, such that epileptogenesis is inhibited in the subject.

Blockers of sodium and/or calcium ion channel activity are well known in the art and can be used as the A moiety in the compounds and methods of the present invention. Similarly, any compound which opens potassium or chloride ion channels can be used as the A moiety in the compounds and methods of the present invention. Antagonist of NMDA receptors and augmenters of endogenous GABA inhibition are also known to one of skill in the art and can be used in the methods and compounds of the invention. For example, 2,3-quinoxalinediones are reported to have NMDA receptor antagonistic activity (see, e.g., U.S. Pat. No. 5,721,234). Exemplary calcium and zinc chelators include moieties known in the art for chelation of divalent cations, including ethylenediaminetetraacetic acid (EDTA), ethylene glycol bis(beta-aminoethyl ether)-N,N,N′,N′-tetraacetic acid, and the like, in addition to those mentioned supra. Exemplary iron chelators include enterobactin, pyridoxal isonicotinyl hydrazones, N,N′-bis(2-hydroxybenzoyl)-ethylenediamine-N,N′-diacetic acid (HBED), and 1-substituted-2-alkyl-3-hydroxy-4-pyridones, including 1-(2′-carboxyethyl)-2-methyl-3-hydroxy-4-pyridone, and other moieties known in the art to chelate iron. Compounds which inhibit NO synthase activity are known in the art and include, e.g., Nγ-substituted arginine analogs, especially of the L configuration, including L-Nγ-nitro-arginine (a specific inhibitor of cerebral NO synthase), L-Nγ-amino-arginine, and L-Nγ-alkyl-arginines; or an ester thereof, preferably the methyl ester. Exemplary antioxidants include ascorbic acid, tocopherols including alpha-tocopherol, and the like.

In another embodiment, the invention provides a method for inhibiting a convulsive disorder. The method includes the step of administering to a subject in need thereof an effective amount of a dioxapiperazine (also known as diketopiperazine) compound represented by the formula (Formula IV):

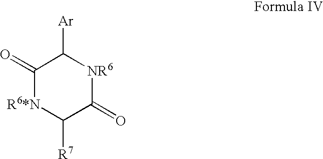

where Ar represents an unsubstituted or substituted aryl group; R⁷ is hydrogen, alkyl, mercaptoalkyl, alkenyl, alkynyl, cycloalkyl, aryl, alkylcarbonyl, arylcarbonyl, alkoxycarbonyl, aryloxycarbonyl, cyano, carboxyl, alkoxycarbonyl, aryloxycarbonyl, or —(CH₂)ₙ—Y, where n is an integer from 1 to 4 and Y is a heterocyclic moiety selected from the group consisting of thiazolyl, triazolyl, and imidazolyl; and R⁶ and R⁶* are each independently hydrogen, alkyl, alkylcarbonyl or arylcarbonyl; or a pharmaceutically acceptable salt thereof; such that the convulsive disorder is inhibited. In a preferred embodiment, R⁷ is not hydrogen, methyl or phenyl. In a preferred embodiment, the compound is cyclo-D-phenylglycyl-(S-Me)-L-cysteine. For synthesis of dioxapiperazines, See, e.g., Kopple, K. D. et al., J. Org. Chem. 33:862 (1968); Slater, G. P. Chem Ind. (London) 32:1092 (1969); Grahl-Nielsen, O. Tetrahedron Lett. 1969:2827 (1969). Synthesis of selected dioxapiperazine compounds is described in the Examples, infra.

In another embodiment, the invention provides a method for concurrently inhibiting epileptogenesis and ictogenesis, the method including the step of administering to a subject in need thereof an effective amount of a compound of the formula:

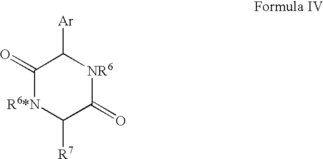

where Ar represents an unsubstituted or substituted aryl group; R⁷ is hydrogen, alkyl, mercaptoalkyl, alkenyl, alkynyl, cycloalkyl, aryl, alkylcarbonyl, arylcarbonyl, alkoxycarbonyl, aryloxycarbonyl, cyano, carboxyl, alkoxycarbonyl, aryloxycarbonyl, or —(CH₂)ₙ—Y, where n is an integer from 1 to 4 and Y is a heterocyclic moiety selected from the group consisting of thiazolyl, triazolyl, and imidazolyl; R⁶ is hydrogen or alkyl, alkylcarbonyl, arylcarbonyl, alkoxycarbonyl or aryloxycarbonyl; and R⁶* is selected from the group consisting of an antioxidant moiety, an NMDA antagonist, an NO synthase inhibitor, an iron chelator moiety, a Ca(II) chelator moiety, a Zn(II) chelator moiety, and an antioxidant moiety; or a pharmaceutically acceptable salt thereof; such that epileptogenesis is inhibited. In certain embodiments, R⁷ is not hydrogen, methyl or phenyl.

In another embodiment, the invention provides a method for treating a disorder associated with NMDA receptor antagonism. The method includes the step of administering to a subject in need thereof an effective amount of a compound of the formula:

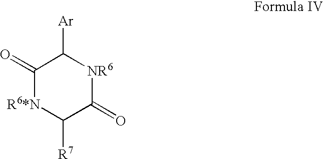

where Ar represents an unsubstituted or substituted aryl group; R⁷ is hydrogen, alkyl, mercaptoalkyl, alkenyl, alkynyl, cycloalkyl, aryl, alkylcarbonyl, arylcarbonyl, alkoxycarbonyl, aryloxycarbonyl, cyano, carboxyl, alkoxycarbonyl, aryloxycarbonyl, or —(CH₂)ₙ—Y, where n is an integer from 1 to 4 and Y is a heterocyclic moiety selected from the group consisting of thiazolyl, triazolyl, and imidazolyl; R⁶ is hydrogen or alkyl, alkylcarbonyl, arylcarbonyl, alkoxycarbonyl or aryloxycarbonyl; and R⁶* is an NMDA antagonist moiety; or a pharmaceutically acceptable salt thereof; such that the disorder associated with NMDA receptor antagonism is treated. In certain embodiments, R⁷ is not hydrogen, methyl or phenyl.

In yet another embodiment, the invention provides a method for inhibiting ictogenesis and epileptogenesis in a subject. The method includes the step of administering to a subject in need thereof an effective amount of an agent represented by the formula A-B, where A is a domain having sodium ion channel blocking activity; and B is a domain having at least one activity selected from the group consisting of NMDA receptor antagonism, GABA inhibition augmentation, calcium binding, iron binding, zinc binding, NO synthase inhibition, and antioxidant activity, such that epileptogenesis is inhibited in the subject. In certain preferred embodiments, the domains A and B (e.g., pharmacophores) of the agent are covalently linked. In certain preferred embodiments, A is a dioxapiperazine moiety, a phenytoin moiety, or a carbamazepine moiety.

In another embodiment, the invention provides a method for inhibiting ictogenesis and epileptogenesis in a subject. The method includes the step of administering to a subject in need thereof an effective amount of an agent represented by the formula A-B, where A is a domain having anti-icotgenenic activity; and B is a domain having at least one activity selected from the group consisting of NMDA receptor antagonism; GABA inhibition augmentation; calcium binding; iron binding; zinc binding; NO synthase inhibition; and antioxidant activity; such that epileptogenesis is inhibited in the subject. In certain preferred embodiments, the domains A and B (e.g., pharmacophores) of the agent are covalently linked. In certain preferred embodiments, A is a dioxapiperazine moiety, a phenytoin moiety, or a carbamazepine moiety.

A hybrid drug according to the invention can be a bifunctional molecule created by connecting an anti-ictogenic moiety with an anti-epileptogenic moiety via, preferably, a covalent linkage such as an amide bond or an ester bond. The linkage can optionally be cleavable in vivo. The linkage can also include a linker or spacer moiety to provide flexibility or sufficient space between the A and B moieties to permit interaction with the respective moieties to which A and B bind or with which A and B interact. Exemplary linkers include diacids (such as adipic acid), e.g., to link amino group-containing A and B moieties; or diamines (such as 1,6-hexanediamine), e.g., to link carboxyl group-containing A and B moieties; or amino acids, e.g., to link an amino-functionalized B moiety to a carboxy-functionalized A moiety (or vice versa). A linker can be selected to provide desired properties according to considerations well known to one of skill in the art. The bifunctional molecule thus targets both ictogenesis and epileptogenesis. The skilled practitioner will appreciate that a hybrid drug may comprise one or more desired average pharmacophores.

In another embodiment, a method for inhibiting epileptogenesis and/or ictogenesis in a subject involves administering to a subject an effective amount of a compound such that epileptogenesis is inhibited, where the compound is of Formula A:

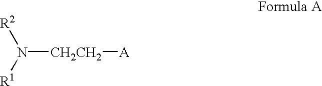

where R¹ is hydrogen, alkyl, alkenyl, alkynyl, aryl, alkylcarbonyl, arylcarbonyl, alkoxycarbonyl, or aryloxycarbonyl; R² is alkyl, alkenyl, alkynyl, aryl, alkylcarbonyl, arylcarbonyl, alkoxycarbonyl, or aryloxycarbonyl; A is an anionic group at physiological pH; and pharmaceutically acceptable salts or esters thereof.

In a preferred embodiment of Formula A, A is carboxylic acid or ester. In another preferred embodiment of Formula A, R¹ is hydrogen. In yet another preferred embodiment of Formula A, R² is alkyl, e.g., arylalkyl such as phenylalkyl.

Examples of compounds of Formula A include

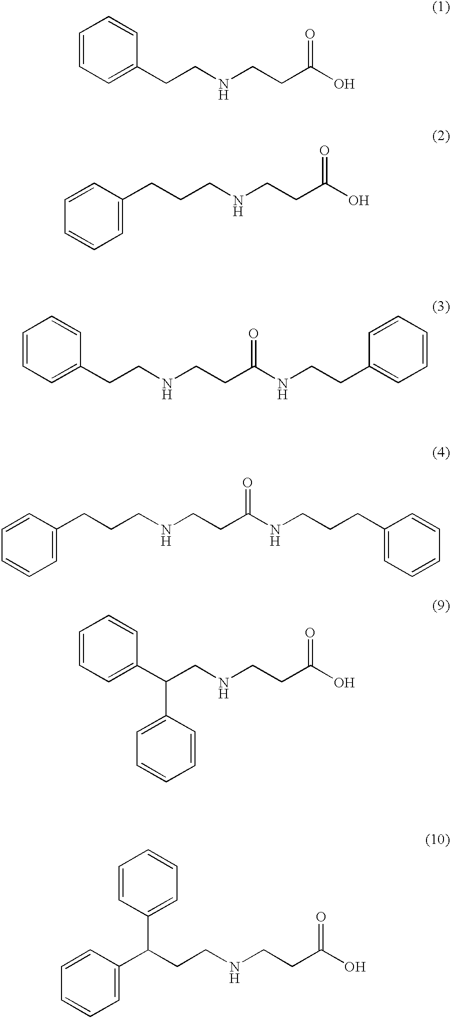

and pharmaceutically acceptable salts or esters thereof.

In another embodiment, a method for inhibiting epileptogenesis and/or ictogenesis in a subject involves administering to a subject an effective amount of a compound such that epileptogenesis is inhibited, where the compound is of Formula B:

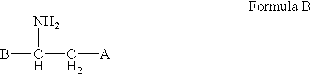

wherein A is an anionic group at physiological pH; B is a phenoxy substituted phenyl group; and pharmaceutically acceptable salts or esters thereof.

In a preferred embodiment of Formula B, A is a carboxyl group. In preferred embodiments of Formula B, B is an alkylphenoxy substituted phenyl group, e.g., a methylphenoxy substituted phenyl group, or a halophenoxy substituted phenyl group, e.g., a chlorophenoxy substituted phenyl group. Preferably compounds of Formula B are a single stereoisomer, as exemplified hereinbelow.

Examples of compounds of Formula B include

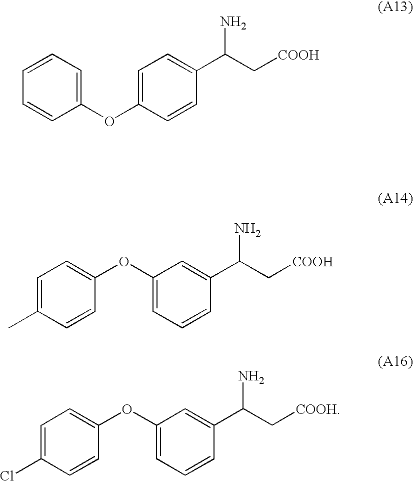

and pharmaceutically acceptable salts or esters thereof.

Still further preferred embodiments of compounds of Formula B are presented in Table 5, and below:

| | |
|---|---|
| (R)-3-Amino-3-[3-(3- trifluoromethylphenoxy)phenyl]propionic acid hydrochloride | 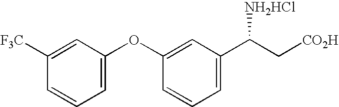 |
| (S)-3-Amino-3-[3- (trifluoromethylphenoxy)phenyl]propionic acid hydrochloride | 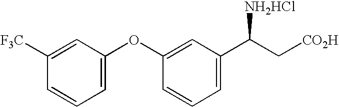 |
| (R)-3-Amino-3-[3-(4- methylphenoxy)phenyl]propionic acid hydrochloride | 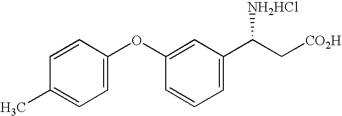 |
| (S)-3-Amino-3-[3-(4- methylphenoxy)phenyl]propionic acid hydrochloride | 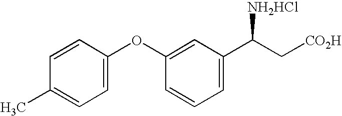 |
| (R)-3-Amino-3-[3- (phenoxy)phenyl]propionic acid hydrochloride | 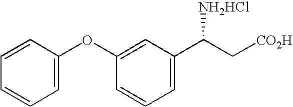 |
| (S)-3-Amino-3-[3- (phenoxy)phenyl]propionic acid hydrochloride | 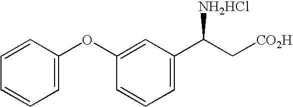 |
| (D)-(+)-3-amino-3-[3-(4- chlorophenoxy)phenyl] propionic acid, hydrochloride | 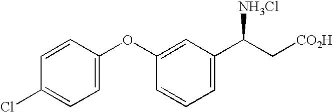 |
| (L)-(−)-3-amino-3-[3-(4- chlorophenoxy)phenyl]propionic acid, hydrochloride | 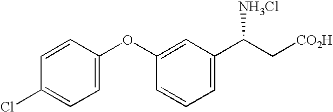 |
| (L)-(−)-3-amino-3-[3-(3,4- dichlorophenoxy)phenyl]propionic acid, hydrochloride | 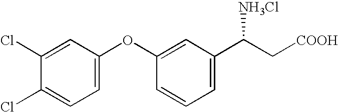 |
| (D)-(+)-3-amino-3-[3-(3,4- dichlorophenoxy)phenyl]propionic acid, hydrochloride | 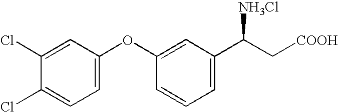 |
| 3-amino-3-(3- phenoxy)phenylpropionic acid, hydrochloride | 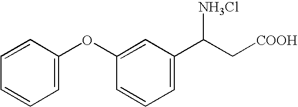 |

In another embodiment, a method for inhibiting epileptogenesis and/or ictogenes is in a subject involves administering to a subject an effective amount of a compound such that epileptogenesis is inhibited, where the compound is of Formula C:

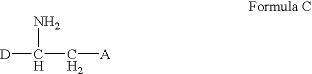

where A is an anionic group at physiological pH; D is an aryl group substituted with 2 or more alkoxy or aryloxy moieties; and pharmaceutically acceptable salts or esters thereof.

In a preferred embodiment of Formula C, A is a carboxyl group. In another preferred embodiment of Formula C, D is a phenyl group substituted with 2 or more alkoxy or aryloxy moieties. In another preferred embodiment of Formula C, D is a phenyl group substituted with 2 or more alkoxy (e.g., methoxy) groups.

Examples of compounds of Formula C include

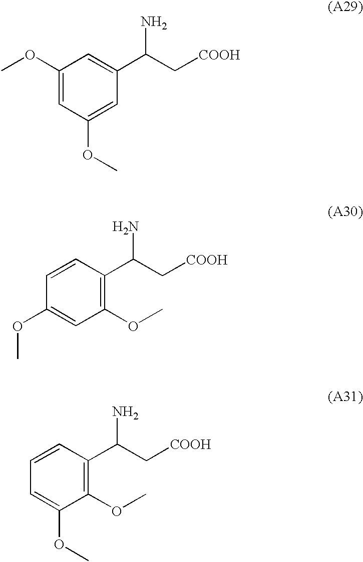

and pharmaceutically acceptable salts thereof.

In another embodiment, a method for inhibiting epileptogenesis and/or ictogenesis in a subject, comprises administering to a subject an effective amount of a compound such that epileptogenesis is inhibited, where the compound is of Formula D

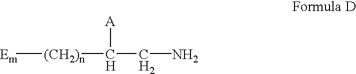

where A is an anionic group at physiological pH; m and n are 1 to 3; E is a substituted or unsubstituted phenyl, and pharmaceutically acceptable salts or esters thereof.

In a preferred embodiment of Formula D, A is a carboxyl group. In another preferred embodiment of Formula D, n is 1 and E is a diphenyl substituted methyl.

Examples of compounds of Formula D include

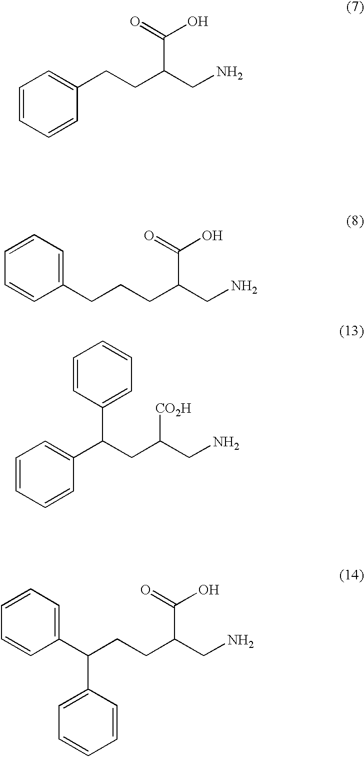

and pharmaceutically acceptable salts or esters thereof.

In yet another embodiment, a method for inhibiting epileptogenesis and/or ictogenesis in a subject, comprises administering to a subject an effective amount of a compound such that epileptogenesis is inhibited, where the compound is of Formula E

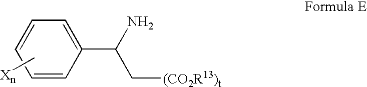

where R¹³ is a hydrogen, alkyl, aryl, or an organic or inorganic salt-forming cation; n is 1 to 5; t is 1 to 2 (preferred); each X is independently selected from the group consisting of halogen, nitro, cyano, and substituted or unsubstituted alkyl and alkoxy groups; and pharmaceutically acceptable salts or esters thereof.

In a preferred embodiment of Formula E, R¹³ is an hydrogen and t is 2.

Examples of preferred compounds of Formula E include the following:

| | |
|---|---|
| 3-Amino-3-(4-nitrophenyl) propionic acid | 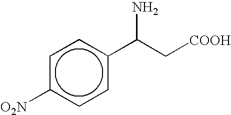 |
| 3-Amino-3-(4-methylphenyl)- 2-carboxypropionic acid acid | 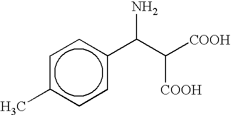 |
| 3-Amino-3-(4-methoxy- phenyl)-2- carboxypropionic acid | 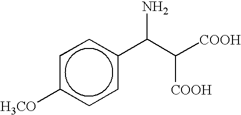 |
| 3-Amino-3-(4-nitrophenyl)-2- carboxypropionic acid | 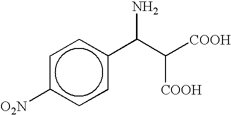 |

Compounds which find use in the therapeutic methods of the invention can be determined through routine screening assays. For example, the animal model of Phase 1 epileptogenesis described in Example 2, infra, can be employed to determine whether a particular compound has anti-epileptogenic activity against Phase 1 epileptogenesis. Chronic epileptogenesis can be modeled in rats (and candidate compounds screened with) the kindling assay described by Silver et al. (Ann. Neurol. (1991) 29:356). Similarly, compounds useful as anticonvulsants can be screened in conventional animal models, such as the mouse model described in Horton, R. W. et al., Eur. J. Pharmacol. (1979) 59:75-83. Compounds or pharmacophores useful for, e.g., binding to or inhibition of receptors or enzymes can be screened according to conventional methods known to the ordinarily skilled practitioner. For example, binding to the GABA uptake receptor can be quantified by the method of Ramsey et al. as modified by Schlewer (Schlewer, J., et al., J. Med. Chem. (1991) 34:2547). Binding to the glycine site on an NMDA receptor can be quantified, e.g., according to the method described in Kemp, A., et al., Proc. Natl. Acad. Sci. USA (1988) 85:6547. Effect on the voltage-gated Na⁺ channel can be evaluated in vitro by voltage clamp assay in rat hippocampal slices.

Assays suitable for screening candidate compounds for anticonvulsive and/or anti-epileptogenic activity in mice or rats are described in Examples 4 and 5, infra.

II. Compounds and Methods of Identifying Compounds

In another aspect, the invention provides compounds useful for the treatment of epilepsy and convulsive disorders.

In one embodiment, the invention provides an anti-epileptogenic compound of the formula (Formula I)

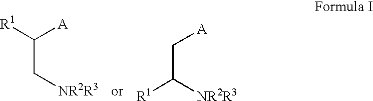

where A is an anionic group at physiological pH; R¹ is alkyl, alkenyl, alkynyl, cycloalkyl, aryl, alkoxy, aryloxy, alkylcarbonyl, arylcarbonyl, alkoxycarbonyl, aryloxycarbonyl, amino, hydroxy, cyano, halogen, carboxyl, alkoxycarbonyloxy, aryloxycarbonyloxy or aminocarbonyl; and R² and R³ are each independently hydrogen, alkyl, alkenyl, alkynyl, cycloalkyl, aryl, alkylcarbonyl, arylcarbonyl, alkoxycarbonyl, or aryloxycarbonyl; or R² and R³, taken together with the nitrogen to which they are attached, form an unsubstituted or substituted heterocycle having from 3 to 7 atoms in the heterocyclic ring; or a pharmaceutically acceptable salt or ester thereof; wherein the anti-epileptogenic compound has anti-epileptogenic activity.

In certain preferred embodiments, A represents carboxylate. In certain preferred embodiments, the compound is selected from the group consisting of α-cyclohexyl-β-alanine, α-(4-tert-butylcyclohexyl)-β-alanine, α-(4-phenylcyclohexyl)-β-alanine, α-cyclododecyl-β-alanine, β-(p-methoxyphenethyl)-β-alanine, β-(p-methylphenethyl)-β-alanine, and pharmaceutically acceptable salts thereof. In other preferred embodiments, the compound is selected from the group consisting of β-(4-trifluoromethylphenyl)-β-alanine and β-[2-(4-hydroxy-3-methoxyphenyl)ethyl]-β-alanine and pharmaceutically acceptable salts thereof. In still other embodiments, the compound is selected from the group consisting of β-(3-pentyl)-β-alanine and β-(4-methylcyclohexyl)-β-alanine and pharmaceutically acceptable salts thereof.

In another embodiment, the invention provides a dioxapiperazine compound of the formula (Formula IV)

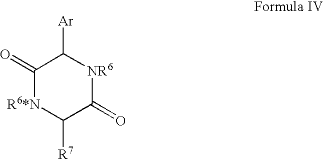

where Ar represents an unsubstituted or substituted aryl group; R⁷ is hydrogen, alkyl, mercaptoalkyl, alkenyl, alkynyl, cycloalkyl, aryl, alkylcarbonyl, arylcarbonyl, alkoxycarbonyl, aryloxycarbonyl, cyano, carboxyl, alkoxycarbonyl, aryloxycarbonyl, or —(CH₂)ₙ—Y, where n is an integer from 1 to 4 and Y is hydrogen or a heterocyclic moiety selected from the group consisting of thiazolyl, triazolyl, and imidazolyl; and R⁶ and R⁶* are each independently hydrogen, alkyl, alkylcarbonyl, arylcarbonyl, alkoxycarbonyl or aryloxycarbonyl; or a pharmaceutically acceptable salt thereof. In some preferred embodiments, the carbon atom to which the Ar group is attached has the “D” or “R” stereochemical configuration. In certain embodiments, Ar is an unsubstituted or substituted phenyl group. In certain embodiments, Y is hydrogen. In certain preferred embodiments, at least one of R⁶ and R⁶* is selected from the group consisting of an antioxidant moiety, an NMDA antagonist, an NO synthase inhibitor, an iron chelator moiety, a Ca(II) chelator moiety, and a Zn(II) chelator moiety. In certain preferred embodiments, R⁷ is methyl or mercaptomethyl.

In certain preferred embodiments, R⁶ and R⁶* are both hydrogen. In certain particularly preferred embodiments, the compound is cyclophenylglycyl-2-(amino-3-mercaptobutanoic acid), more preferably cyclo-β-phenylglycyl-L-[2-(amino-3-mercaptobutanoic acid)]. In a referred embodiment, the compound is cyclo-β-phenylglycyl-(S-Me)-L-cysteine. In some preferred embodiments, Ar is an unsubstituted phenyl group. In certain embodiments, R⁷ is not hydrogen, methyl or phenyl.

In another embodiment, the invention provides a compound of the formula (Formula IV)

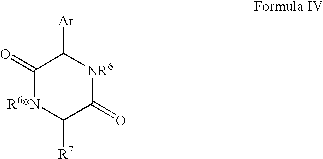

where Ar represents an unsubstituted or substituted aryl group; R⁷ is alkyl, mercaptoalkyl, alkenyl, alkynyl, cycloalkyl, aryl, alkylcarbonyl, arylcarbonyl, alkoxycarbonyl, aryloxycarbonyl, cyano, carboxyl, alkoxycarbonyl, aryloxycarbonyl, or —(CH₂)ₙ—Y, where n is an integer from 1 to 4 and Y is hydrogen or a heterocyclic moiety selected from the group consisting of thiazolyl, triazolyl, and imidazolyl; R⁶ is hydrogen or alkyl, alkylcarbonyl, arylcarbonyl, alkoxycarbonyl or aryloxycarbonyl; and R⁶* is selected from the group consisting of an antioxidant moiety, an NMDA antagonist, an NO synthase inhibitor, an iron chelator moiety, a Ca(II) chelator moiety, and a Zn(II) chelator moiety; or both R⁶ and R⁶* are selected from the group consisting of an antioxidant moiety, an NMDA antagonist, an NO synthase inhibitor, an iron chelator moiety, a Ca(II) chelator moiety, and a Zn(II) chelator moiety; or a pharmaceutically acceptable salt thereof. In certain preferred embodiments, R⁶* is D-α-aminoadipyl. In certain preferred embodiments, R⁷ is mercaptomethyl. In certain embodiments, R⁷ is not hydrogen, methyl or phenyl. In certain preferred embodiments, R⁶* further comprises a cleavable linkage. In one embodiment, the compound comprises cyclo-D-phenylglycyl-L-alanine.

As will be appreciated by the skilled practitioner, the compounds of the invention include compounds which can have a single pharmacophore (e.g., dioxapiperazines where the dioxapiperazine moiety is the sole pharmacophore); or β-amino anionic moieties where the β-amino anionic moiety is responsible for the biochemical activity of the compound. Certain compounds of the invention include two distinct pharmacophores and have a structure represented by A-B, where A and B are each domains or pharmacophores having biochemical activity (e.g., an anticonvulsant dioxapiperazine moiety having a distinct antioxidant moiety, e.g., R⁶*) (also referred to herein as a “hybrid” drug). A compound which includes two pharmacophores can be capable of interaction with two or more distinct receptors. Where the compound of the invention includes more than one pharmacophore, the pharmacophores can be linked to each other by a variety of techniques known to the skilled practitioner. For example, the pharmacophore represented by R⁶* can be covalently bonded to a dioxapiperazine moiety through an amide linkage to a nitrogen of the dioxapiperazine ring. A linkage between two pharmacophores can be selected such that the two pharmacophores are cleaved from each other in vivo (i.e., by the selection of a linkage which is labile in vivo). Examples of such biologically labile linkages are known in the art. See, e.g., Silverman, cited above. Advantageously, such a “hybrid” two-pharmacophore drug can be designed to be transported within the body to reach a site or organ such as the brain, where one or more pharmacophore moieties exert a biological effect, at which site the hybrid drug can be cleaved to provide two active drug moieties. Some examples of hybrid drugs are set forth above.

The invention further contemplates the use of prodrugs which are converted in vivo to the therapeutic compounds of the invention. Such prodrugs can be used to alter the biodistribution (e.g., to allow compounds which would not typically cross the blood-brain barrier to cross the blood-brain barrier) or the pharmacokinetics of the therapeutic compound. For example, an anionic group, e.g., a carboxylate or sulfonate, can be esterified, e.g, with a methyl group or a phenyl group, to yield a carboxylate or sulfonate ester. When the carboxylate or sulfonate ester is administered to a subject, the ester is cleaved, enzymatically or non-enzymatically, to reveal the anionic group. Such an ester can be cyclic, e.g., a lactone or sultone, or two or more anionic moieties may be esterified through a linking group. An anionic group can be esterified with moieties (e.g., acyloxymethyl esters) which are cleaved to reveal an intermediate compound which subsequently decomposes to yield the active compound. Alternatively, an anionic moiety can be esterified to a group which is actively transported in vivo, or which is selectively taken up by target organs. The ester can be selected to allow specific targeting of the therapeutic moieties to particular organs. In another embodiment, the prodrug is a reduced form of an anionic group, e.g., a carboxylate or sulfonate, e.g., an alcohol or thiol, which is oxidized in vivo to the therapeutic compound.

Thus, as described above, preferred compounds include pyrimidines, such as substituted uracils, which can be converted in vivo to β-amino anionic compounds. In a preferred embodiment, the compound can be represented by the formula (Formula V):

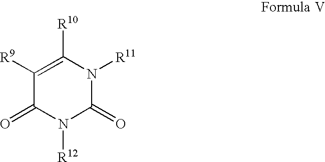

where R⁹ and R¹⁰ are each independently selected from the group consisting of hydrogen, alkyl (including cycloalkyl, heterocyclyl, and aralkyl), alkenyl, alkynyl, aryl, alkoxy, aryloxy, alkylcarbonyl, arylcarbonyl, alkoxycarbonyl, aryloxycarbonyl, amino (including unsubstituted and substituted amino), hydroxy, thiol, alkylthiol, nitro, cyano, halogen, carboxyl, alkoxycarbonyloxy, aryloxycarbonyloxy or aminocarbonyl; or R⁹ and R¹⁰, together with the two-carbon unit to which they are attached, are joined to form a carbocyclic or heterocyclic ring having from 4 to 8 members in the ring; and R¹¹ is hydrogen, alkyl, alkenyl, alkynyl, cycloalkyl, aryl, alkylcarbonyl, arylcarbonyl, alkoxycarbonyl, or aryloxycarbonyl; or R¹⁰ and R¹¹, together with the carbon atom and nitrogen atom to which they are respectively attached, are joined to form a heterocyclic ring having from 4 to 8 members in the ring; and R¹² is selected from the group consisting of hydrogen, alkyl, aryl and a carbohydrate (such as a sugar like ribose or deoxyribose); or a pharmaceutically acceptable salt or ester thereof. In another embodiment, the compound can be represented by the formula (Formula Va):

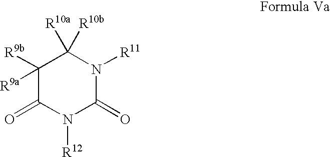

where R^{9a}, R^{9b}, R^{10a}, R^{10b} are each independently selected from the group consisting of hydrogen, alkyl (including cycloalkyl, heterocyclyl, and aralkyl), alkenyl, alkynyl, aryl, alkoxy, aryloxy, alkylcarbonyl, arylcarbonyl, alkoxycarbonyl, aryloxycarbonyl; amino (including unsubstituted and substituted amino), hydroxy, thiol, alkylthiol, nitro, cyano, halogen, carboxyl, alkoxycarbonyloxy, aryloxycarbonyloxy or aminocarbonyl; or R^{9a} and R^{9b}, together with the two-carbon unit to which they are attached, are joined to form a carbocyclic or heterocyclic ring having from 4 to 8 members in the ring; or R^{10a} and R^{10b}, together with the two-carbon unit to which they are attached, are joined to form a carbocyclic or heterocyclic ring having from 4 to 8 members in the ring; or one of R^{9a} and R^{9b} is joined with one of R^{10a} and R^{10b}, together with the two-carbon unit to which they are attached, to form a carbocyclic or heterocyclic ring having from 4 to 8 members in the ring; R¹¹ is hydrogen, alkyl, alkenyl, alkynyl, cycloalkyl, aryl, alkylcarbonyl, arylcarbonyl, alkoxycarbonyl, or aryloxycarbonyl; or one of R^{10a} and R^{10b} is joined with R¹¹, together with the carbon atom and nitrogen atom to which they are respectively attached, to form a heterocyclic ring having from 4 to 8 members in the ring; and R¹² is selected from the group consisting of hydrogen, alkyl, aryl and a carbohydrate (such as a sugar, e.g., ribose or deoxyribose); or a pharmaceutically acceptable salt or ester thereof.

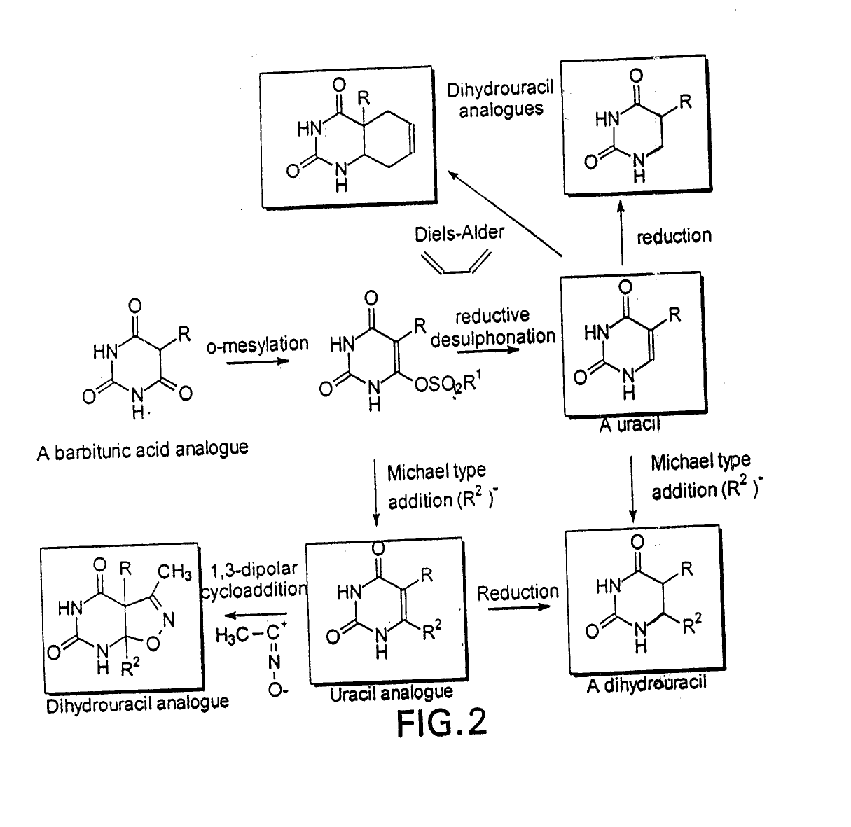

Compounds of Formulas V and Va can be prepared according to a variety of synthetic procedures, some of which are known in the art. Exemplary syntheses are shown in FIG. 2. For example, as shown in FIG. 2, a barbituric acid compound can be modified (e.g., by mesylation with mesyl chloride and an amine base) to provide a compound which can be further functionized (e.g., by Michael addition of a suitable nucleophile); or can be reductively desulphonated to provide a dienophile for subsequent Diels-Alder cycloaddition with a suitable dienophile. Reduction of the uracil ring provides dihydrouracil derivatives.

Compounds useful in the present invention may also include carrier or targeting moieties which allow the therapeutic compound to be selectively delivered to a target organ or organs. For example, if delivery of a therapeutic compound to the brain is desired, the compound may include a moiety capable of targeting the compound to the brain, by either active or passive transport (a “targeting moiety”). Illustratively, the carrier molecule may include a redox moiety, as described in, for example, U.S. Pat. Nos. 4,540,564 and 5,389,623. These patents disclose drugs linked to dihydropyridine moieties which can enter the brain, where they are oxidized to a charged pyridinium species which is trapped in the brain. Thus, drug accumulates in the brain. Other carrier moieties include compounds, such as amino acids or thyroxine, which can be passively or actively transported in vivo. Such a carrier moiety can be metabolically removed in vivo, or can remain intact as part of an active compound. Many targeting moieties are known, and include, for example, asialoglycoproteins (see, e.g., U.S. Pat. No. 5,166,320) and other ligands which are transported into cells via receptor-mediated endocytosis.

The targeting and prodrug strategies described above can be combined to produce a compound that can be transported as a prodrug to a desired site of action and then unmasked to reveal an active compound.

In another aspect, the present invention provides pharmacophore modeling methods for identifying compounds which can inhibit epileptogenesis in a subject. These methods feature the examination of the structures of two or more compounds which are known to cause a direct or indirect pharmacological effect on a protein or a molecule which is involved in epileptogenesis. These proteins and molecules which are involved in epileptogenesis are believed to include cell-surface receptor molecules (e.g., an NMDA receptor) or a molecule that is involved in transport of neurotransmitters (e.g., a GABA transporter). Preferably, the structures of these compounds each include one or more pharmacophores which can exert at least some of the pharmacological effect of the compound. The methods of the invention also include determining average pharmacophore structure(s) (e.g., carbon backbone structures and/or a three-dimensional space filling structures) based on the pharmacophore structures of the two or more compounds. New compounds having one or more of the average pharmacophore structures can be chosen using these methods.

In related embodiments, these methods feature the examination of the structures of two or more compounds which are known to cause a direct or indirect pharmacological effect on two or more proteins or molecules which are involved in epileptogenesis. In such an embodiment, the skilled practitioner will realize that the new compound which is chosen will preferably have one or more pharmacophores which are active on different proteins or molecules involved with epileptogenesis.

In a preferred embodiment, a new compound which is chosen (e.g., designed) by these methods of the invention inhibits epileptogenesis in a subject.

The methods of identifying compounds may further rely on the construction of additional complementary models which simulate at least a portion of a protein or a molecule which is involved in epileptogenesis (e.g., a “pseudoreceptor”). Such a simulation can be used to further evaluate new candidate compounds which comprise one or more average pharmacophores. Complementary models can be constructed using algorithms and/or methods which rely on the structures of pharmacophores or whole compounds that interact with the protein molecule involved with epileptogenesis. Algorithms for the construction of such a simulation will be known to the skilled practitioner and include MM2 molecular mechanics force field (see, e.g., Allinger (1977) J. Am. Chem. Soc. 99:8127-8134, Allinger et al. (1988) J. Comp. Chem. 9:591-595, Lii et al. (1989) J. Comp. Chem. 10:503-513, Cornell et al. (1995) J. Am. Chem. Soc. 117:5179-5197, Wiener et al. (1986) J. Comp. Chem.7:230-252).

The invention further provides a kit which includes a container of a compound of the invention and instructions for using a therapeutically effective amount of the compound to a subject in need thereof such that a convulsive disorder (e.g., epileptogenesis) is inhibited in the subject. The kits of the invention provide convenient means for using, e.g., administering the compounds of the invention. In a particularly preferred embodiment, the kit includes a therapeutically effective amount of the compound, more preferably in unit dosage form.

This invention also provides a method of diagnosing an epileptogenic condition in a subject comprising administering a compound of the invention (e.g. compounds 1-14 and A1-A32 described later) labeled with a detectable marker to said subject; and measuring increased binding of the compound to the NMDA receptors of the neurons of said subject's brain, thereby diagnosing an epileptogenic condition in said subject.

This invention further provides a method of diagnosing an epileptogenic condition in a subject comprising administering a compound of the invention (e.g. compounds 1-14 and A1-A32 described later) labeled with a detectable marker to said subject; and measuring decreased binding of the compound to the GABA receptors of the neurons of said subject's brain, thereby diagnosing an epileptogenic condition in said subject.

“Compound labeled with a detectable marker” as used herein includes compounds that are labeled by a detectable means and includes enzymatically, radioactively, fluorescently, chemiluminescently, and/or bioluminescently labeled antibodies.

Examples of enzymes that can be used as labeled include malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholinesterase.

Examples of radioactive labels include: ³H, ¹²⁵I, ¹³¹I, ³⁵S, ¹⁴C, and preferably ¹²⁵I. Examples of fluoroscent labels include: fluorescein isothiocyanate, rhodamine, phycoerytherin, phycocyanin, allophycocyanin, o-phthaldehyde and fluorescamine. Examples of chemiluminescent labels include: luminol, luciferin, isoluminol, theromatic acridinium ester, imidazole, acridinium salt and oxalate ester. Examples of bioluminescent labels include: luciferin, luciferase and aequorin.

III. Methods for Preparing β-amino Anionic Compounds

The invention further provides methods for preparing β-amino anionic compounds.

In one embodiment, the invention comprises a method for preparing a β-amino carboxyl compound represented by the formula (Formula VI):

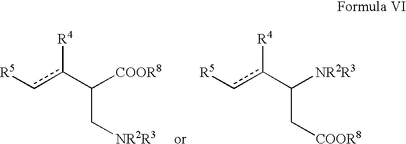

where the dashed line represents an optional single/double bond (of either E- or Z-configuration); R² and R³ are each independently hydrogen, alkyl, alkenyl, alkynyl, cycloalkyl, aryl, alkylcarbonyl, arylcarbonyl, alkoxycarbonyl, or aryloxycarbonyl; or R² and R³, taken together with the nitrogen to which they are attached, form an unsubstituted or substituted heterocycle having from 3 to 7 atoms in the heterocyclic ring; and R⁴ and R⁵ are each independently hydrogen, alkyl, alkenyl, alkynyl, cycloalkyl, aryl, alkylcarbonyl, arylcarbonyl, alkoxycarbonyl, aryloxycarbonyl, amino, hydroxy, cyano, alkoxy, aryloxy, carboxyl, alkoxycarbonyl, aryloxycarbonyl, heterocyclyl; or R⁴ and R⁵, taken together, form a substituted or unsubstituted carbocyclic or heterocyclic ring having from 5 to 15 atoms (more preferably 5 to 8) in the ring; and R⁸ is hydrogen, alkyl, aryl, or an organic or inorganic salt-forming cation. The method includes the steps of reacting a compound of formula VI

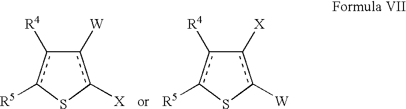

where the dashed lines each represent an optional single/double bond; X is nitro, azido, or NR²R³, wherein R² and R³ are defined above; W is —CN or —COOR⁸; R⁸ is hydrogen, alkyl, aryl, or an organic or inorganic salt-forming cation; and R⁴ and R⁵ are as defined above; under reductive desulfurization conditions such that the β-amino carboxyl or β-amino nitrile compound is formed. In certain preferred embodiments, R² is alkylcarbonyl, arylcarbonyl, alkoxycarbonyl, or aryloxycarbonyl, and R³ is hydrogen.

Compounds of Formula VII can be prepared according to methods known in the art. For example, the synthesis of aminothiophene carboxylates (i.e., the compound of Formula VI where W is —COOR⁸ and R⁸ is a cation, X is an amino group, and each dashed line is a single bond) has been reported by several methods. See, e.g., Beck, J. Org. Chem. (1972) 37:3224; Meth-Cohn, J. Chem. Res. (1977) (S)294, (M)3262. Reduction of aminothiophene carboxylates (or aminothiophene nitrites) under reductive desulfurization conditions has now been found to produce β-amino acids in good yield (aminothiophene nitrites also require hydrolysis of the nitrile group, which can be accomplished according to well-known methods. See, e.g., Larock, Comprehensive Organic Transformations, VCH Publishers (1989), and references cited therein. In a preferred embodiment, the reductive desulfurization conditions comprise reacting the aminothiophene carboxylate with Raney nickel, such that the aminothiophene carboxylate is desulfurized.

In another embodiment, the invention provides a method for preparing a β-amino carboxyl compound represented by formula VIII:

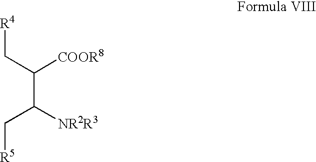

where R² and R³ are each independently hydrogen, alkyl, alkenyl, alkynyl, cycloalkyl, aryl, alkylcarbonyl, arylcarbonyl, alkoxycarbonyl, or aryloxycarbonyl; or R² and R³, taken together with the nitrogen to which they are attached, form an unsubstituted or substituted heterocycle having from 3 to 7 atoms in the heterocyclic ring; and R⁴ and R⁵ are each independently hydrogen, alkyl, alkenyl, alkynyl, cycloalkyl, aryl, alkylcarbonyl, arylcarbonyl, alkoxycarbonyl, aryloxycarbonyl, amino, hydroxy, cyano, alkoxy, aryloxy, carboxyl, alkoxycarbonyl, aryloxycarbonyl, heterocyclyl; or R⁴ and R⁵, taken together, form a substituted or unsubstituted carbocyclic or heterocyclic ring having from 5 to 15 atoms (more preferably 5 to 8 atoms) in the ring; and R⁸ is hydrogen, alkyl, aryl, or an organic or inorganic salt-forming cation. The method includes the steps of reacting a compound of formula IX

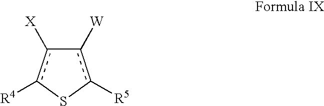

where the dashed lines each represent an optional single bond; X is nitro, azido, or NR²R³, wherein R² and R³ are defined above; W is —CN or —COOR⁸; R⁸ is hydrogen, alkyl, aryl, or an organic or inorganic salt-forming cation; and R⁴ and R⁵ are as defined above; under reductive desulfurization conditions such that the β-amino carboxyl compound of Formula VIII is formed (where W=—CN, the carboxylate will be formed after reductive desulfurization and acidification). In certain preferred embodiments, R² is alkylcarbonyl, arylcarbonyl, alkoxycarbonyl, or aryloxycarbonyl, and R³ is hydrogen.

Compounds of Formula IX (or esters thereof, which can be hydrolyzed according to known methods to provided compounds of Formula IX) can be prepared according to methods known in the art. See, e.g., U.S. Pat. No. 4,029,647; Henriksen and Autrup, Acta Chem. Scand. 26:3342 (1972); or Hartke and Peshkar, Pharm. Zentralhalle 107:348 (1968).

The synthetic methods of the invention provide advantages over previously reported syntheses of β-amino acids. For example, the inventive methods provide access to a variety of β-amino acids substituted at either carbon, or both carbons, of the two-carbon backbone; the particular β-amino acid produced is determined by the starting aminothiophene carboxylate, which can be prepared with a variety of substituents. As described in Example 1, infra, the inventive methods provide β-amino acids in good yield, under mild conditions, and in only a small number of steps from commercially available reagents. Illustrative compounds which have been prepared by this method are presented in Example 1. The methods of the invention thus provide a general, rapid, simple, and high-yielding route to β-amino acids.

In another embodiment, the invention provides a method for preparing a β-aryl-β-alanine compound. In this embodiment, the invention provides a simple, one-pot reaction capable of producing a variety of substituted and unsubstituted β-aryl-β-alanine compounds, often using readily available precursors. The method used herein is an adaptation to produce β-alanine analogs. The method includes the steps of reacting an aryl aldehyde with a malonate compound and an ammonium compound, under conditions such that a β-aryl-β-alanine compound is formed. In a preferred embodiment, the aryl aldehyde is a substituted or unsubstituted benzaldehyde. In a preferred embodiment, the malonate compound is malonic acid. In a preferred embodiment, the ammonium compound is an ammonium salt of a compound selected from the group consisting of ammonia, primary amines, and secondary amines. A particularly preferred ammonium compound is a salt of ammonia, most preferably ammonium acetate. In a preferred embodiment, the solvent is a polar organic solvent such as ethanol. An exemplary synthesis according to the invention is described in Example 3.

It will be appreciated that β-amino acids, in addition to the anti-epileptogenic properties described herein, have other uses, e.g., as synthetic intermediates and as commodity chemicals. For example, the β-lactam structure is present in many commercially-valuable antibiotics, including, for example, penicillins, carbapenems, norcardins, monobactams, and the like. A variety of methods for conversion of β-amino acids to β-lactams have been reported. See, e.g., Wang, W.-B. and Roskamp, E. J., J. Am. Chem. Soc. (1993) 115:9417-9420 and references cited therein. Thus, the present invention further provides a method for the synthesis of β-lactams. The method comprises subjecting a compound of Formula VII (or Formula IX) to reductive desulfurization conditions to produce a compound of Formula VI (or I or VIII), followed by cyclization of the compound of Formula VI (or I or VIII) to form a β-lactam. Moreover, β-amino acids have been shown to improve the condition of certain cancer patients (see, e.g., Rougereau, A. et al. Ann. Gastroenterol. Hepatol. (Paris) 29 (2): 99-102 (1993). Thus, the present invention provides methods for preparing compounds useful for the treatment of cancer.

IV. Libraries

In another aspect, the invention provides libraries of compounds of Formula IV, Formula VI, or Formula VIII, and methods of preparing such libraries.

The synthesis of combinatorial libraries is well known in the art and has been reviewed (see, e.g., E. M. Gordon et al., J. Med. Chem. 37:1385-1401 (1994)). Thus, the invention includes methods for synthesis of combinatorial libraries of compounds of Formula IV, Formula VI, or Formula VIII. Such libraries can be synthesized according to a variety of methods. For example, a “split-pool” strategy can be implemented to produce a library of compounds. The library of immobilized compounds can then be washed to remove impurities. In certain embodiments, the immobilized compounds can be cleaved from the solid support to yield a compound of Formula IV, VI, or VIII.

In another illustrative method of combinatorial synthesis, a “diversomer library” is created by the method of Hobbs, DeWitt et al. (Proc. Natl. Acad. Sci. U.S.A. 90:6909 (1993)). After creation of the library of compounds, purification and workup yields a soluble library of substituted compounds of Formula IV, VI, or VIII.

Other synthesis methods, including the “tea-bag” technique of Houghten et al., Nature 354:84-86 (1991), can also be used to synthesize libraries of compounds according to the subject invention.

Combinatorial libraries can be screened to determine whether any members of the library have a desired activity, and, if so, to identify the active species. Methods of screening combinatorial libraries have been described (see, e.g., Gordon et al., J Med. Chem., op. cit.). Soluble compound libraries can be screened by affinity chromatography with an appropriate receptor to isolate ligands for the receptor, followed by identification of the isolated ligands by conventional techniques (e.g., mass spectrometry, NMR, and the like). Immobilized compounds can be screened by contacting the compounds with a soluble receptor; preferably, the soluble receptor is conjugated to a label (e.g., fluorophores, calorimetric enzymes, radioisotopes, luminescent compounds, and the like) that can be detected to indicate ligand binding. Alternatively, immobilized compounds can be selectively released and allowed to diffuse through a membrane to interact with a receptor. Exemplary assays useful for screening the libraries of the invention are known in the art (see, e.g., E. M. Gordon et al., J. Med. Chem. 37:1385-1401 (1994)).

Combinatorial libraries of compounds can also be synthesized with “tags” to encode the identity of each member of the library. see, e.g., U.S. Pat. No. 5,565,324 and PCT Publication No. WO 94/08051). In general, this method features the use of inert, but readily detectable, tags, that are attached to the solid support or to the compounds. When an active compound is detected such as by one of the techniques described above, the identity of the compound is determined by identification of the unique accompanying tag. This tagging method permits the synthesis of large libraries of compounds which can be identified at very low levels.

In preferred embodiments, the libraries of compounds of the invention contain at least 30 compounds, more preferably at least 100 compounds, and still more preferably at least 500 compounds. In preferred embodiments, the libraries of compounds of the invention contain fewer than 10⁹ compounds, more preferably fewer than 10⁸ compounds, and still more preferably fewer than 10⁷ compounds.

A library of compounds is preferably substantially pure, i.e., substantially free of compounds other than the intended products, e.g., members of the library. In preferred embodiments, the purity of a library produced according to the methods of the invention is at least about 50%, more preferably at least about 70%, still more preferably at least about 90%, and most preferably at least about 95%.

The libraries of the invention can be prepared as described herein. In general, at least one starting material used for synthesis of the libraries of the invention is provided as a variegated population. The term “variegated population”, as used herein, refers to a population including at least two different chemical entities, e.g., of different chemical structure. For example, a “variegated population” of compounds of Formula VII would comprise at least two different compounds of Formula VII. Use of a variegated population of linkers to immobilize compounds to the solid support can produce a variety of compounds upon cleavage of the linkers.

Libraries of the invention are useful for, inter alia, drug discovery. For example, a library of the invention can be screened to determine whether the library includes compounds having a pre-selected activity, e.g., anti-epileptogenic or anticonvulsant activity.

V. Pharmaceutical Compositions

In another aspect, the present invention provides pharmaceutically acceptable compositions which comprise a therapeutically-effective amount of one or more of the compounds described above, formulated together with one or more pharmaceutically acceptable carriers (additives) and/or diluents. The pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes for application to the tongue; (2) parenteral administration, for example, by subcutaneous, intramuscular or intravenous injection as, for example, a sterile solution or suspension; (3) topical application, for example, as a cream, ointment or spray applied to the skin; or (4) intravaginally or intrarectally, for example, as a pessary, cream or foam. In a preferred embodiment, the therapeutic compound is administered orally. The compounds of the invention can be formulated as pharmaceutical compositions for administration to a subject, e.g., a mammal, including a human.

The compounds of the invention are administered to subjects in a biologically compatible form suitable for pharmaceutical administration in vivo. By “biologically compatible form suitable for administration in vivo” is meant a compound to be administered where any toxic effects are outweighed by the therapeutic effects of the antibody. The term subject is intended to include living organisms where an immune response can be elicited, e.g., mammals. Examples of subjects include humans, dogs, cats, rodents (e.g., miceor rats), and transgenic species thereof. Administration of a therapeutically active amount of the therapeutic compositions of the present invention is defined as an amount effective, at dosages and for periods of time necessary to achieve the desired result. For example, a therapeutically active amount of a compound of the invention may vary according to factors such as the disease state, age, sex, and weight of the individual, and the ability of antibody to elicit a desired response in the individual. Dosage regimes may be adjusted to provide the optimum therapeutic response. For example, several divided doses may be administered daily or the dose may be proportionally reduced as indicated by the exigencies of the therapeutic situation.

The active compound may be administered in a convenient manner such as by injection (subcutaneous, intravenous, etc.), oral administration, inhalation, transdermal application, or rectal administration. Depending on the route of administration, the active compound may be coated in a material to protect the compound from the action of enzymes, acids and other natural conditions which may inactivate the compound.

A compound of the invention can be administered to a subject in an appropriate carrier or diluent, co-administered with enzyme inhibitors or in an appropriate carrier such as liposomes. The term “pharmaceutically acceptable carrier” as used herein is intended to include diluents such as saline and aqueous buffer solutions. To administer a compound of the invention by other than parenteral administration, it may be necessary to coat the antibody with, or co-administer the compound with a material to prevent its inactivation. Liposomes include water-in-oil-in-water emulsions as well as conventional liposomes (Strejan et al., (1984) J. Neuroimmunol 7:27). The active compound may also be administered parenterally or intraperitoneally. Dispersions can also be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. In all cases, the composition must be sterile and must be fluid to the extent that easy syringability exists. It must be stable under the conditions of manufacture and storage and must be preserved against the contaminating action of microorganisms such as bacteria and fungi. The pharmaceutically acceptable carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

When the active compound is suitably protected, as described above, the composition may be orally administered, for example, with an inert diluent or an assimilable edible carrier. As used herein “pharmaceutically acceptable carrier” includes any and all solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the active compound, use thereof in the therapeutic compositions is contemplated. Supplementary active compounds can also be incorporated into the compositions.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the therapeutic treatment of individuals.

### EXAMPLES

### Example 1

### Identification of Compounds Based on a Pharmacophore Model

A pharmacophore model was developed which incorporated the structural parameters and features of two different classes of compounds: (1) inhibitors of GABA uptake receptors, and (2) co-agonists of the NMDA receptor.

Previous models (Murali Dhar et al. (1994) J. Med. Hem. 37:2334, Falch and Krogsgaard-Larson (1991) Eur. J. Med. Chem. 26:69, N'Goka (1991) J. Med. Chem. 34:2547) suggest that GABA uptake inhibitors should include:

i) An amine functional group (preferrably a second amine)

ii) A carboxylic functional group

iii) A lipophilic group, preferably aromatic

iv) An electron-rich functionality (double-bond or an oxygen) located between the amine and the lipophilic group

v) A two carbon chain length between the amine functional group and the double bond or the oxygen atom.

Other previous models focused on antagonists of the glycine co-agonist site of the NMDA receptor complex (e.g., Leeson and Iverson (1994) J. Med. Chem. 37:4053) suggest that co-agonists of the NMDA receptor should desirably include:

i) An amine functional group (preferrably a second amine)

ii) A carboxylic functional group

iii) Two small lipophilic groups

iv) A large lipophilic group

Based on this information, average pharmacophore model compounds were prepared which, as a class, may be considered to be β-amino acids and analogs thereof. Important parameters of these compounds include:

i) An amine group

ii) A carboxylic functional group

iii) A β-alanine backbone

iv) A flexible lipophilic moiety

To further refine the profile of desired compounds, a 3-dimensional visualization of an “average receptor site” was constructed using a series of molecular modeling calculations (MM2 molecular mechanics force field). First, using various probe molecules known to bind to the glycine subsite on the NMDA receptor, a “pseudo receptor” model was created using a complementary modeling approach. To achieve this, fragments of the known NMDA receptor site peptides were mathematically positioned in the vicinity of several probe molecules (e.g., compounds known to bind the receptor) to simulate a receptor, i.e., the probe molecules were used as a template to compile a receptor model around them. For example, the side-chain of glutamate was used to “dock” to basic ammonium functionalities in the probe molecule. Lipophilic pockets were simulated with the side-chain of phenylalanine. By doing so, the “receptor” of the glycine subsite on the NMDA receptor was mathematically modeled. Next, the same procedure was carried out for the glial GABA uptake receptor. The two model receptors were than overlapped to design a model hybrid receptor (average receptor site). This model hybrid receptor site contained three “pockets”. An anionic pocket was situated 7.7 Å from a cationic pocket capable of interacting with ammonium and carboxylate functionalities, respectively. A mobile lipophilic pocket was located in a variable position ranging from 5.2 to 8.1 Å from the anionic pocket. β-amino acid analogues which include the above criteria were inserted into the model hybrid receptor. Optimal fit was obtained with β-substituted β-amino acids possessing an aromatic ring on a short (2-3 carbon) flexible arm. The flexible arm appeared to enable interaction with the mobile lipophilic pocket.

A list of candidate compounds which were identified by these methods is given below.

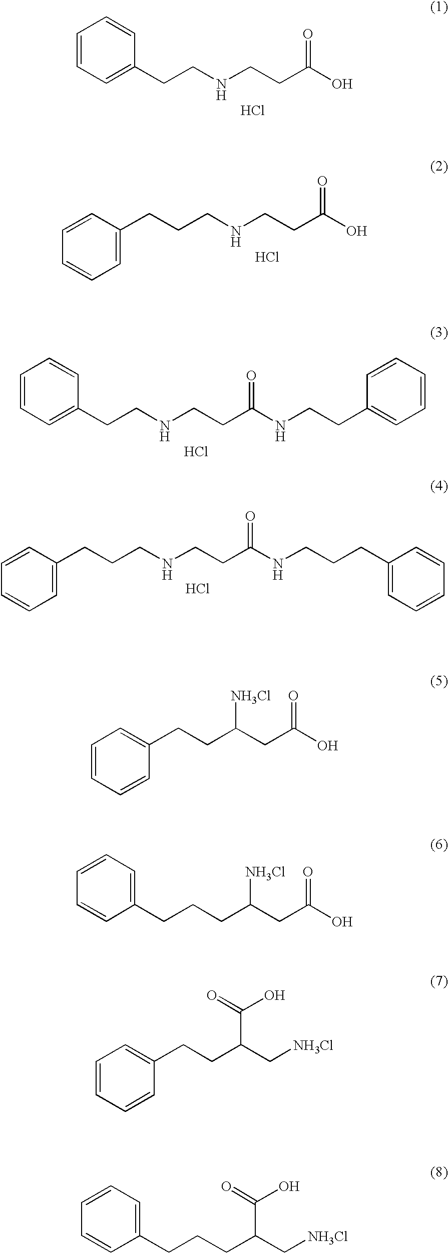

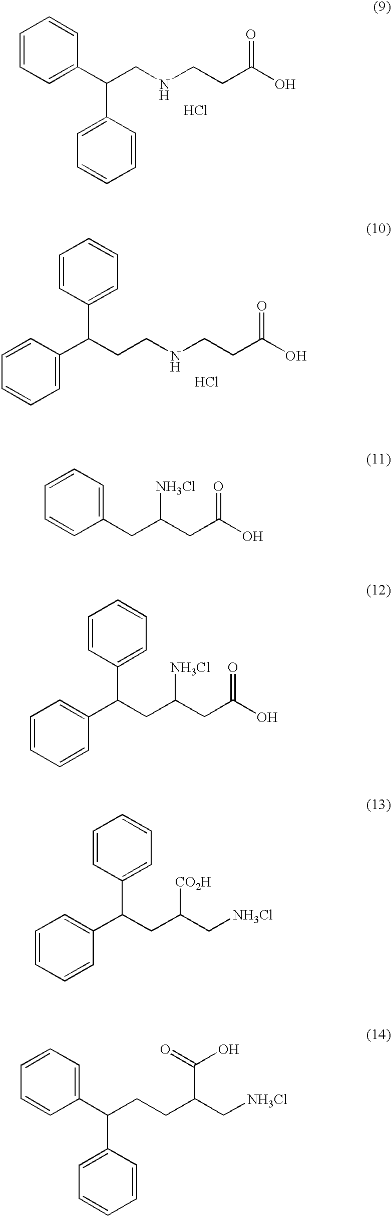

A number of β-aryl β-amino-acid compounds were further produced by a facile “one pot” synthesis method. In brief, to a solution of a substituted benzaldehyde in absolute ethanol was added malonic acid and excess ammonium acetate, and the reaction mixture was heated to reflux. The reaction mixture was cooled to yield a mixture of the β-aryl β-alanine and (in certain cases) a cinnamic acid derivative. The cinnamic acid (if present) was removed by acid/base extraction of the mixture to yield the β-aryl-β-alanine, often in moderate to good yield. A list of candidate compounds which were obtained by this method are listed below.

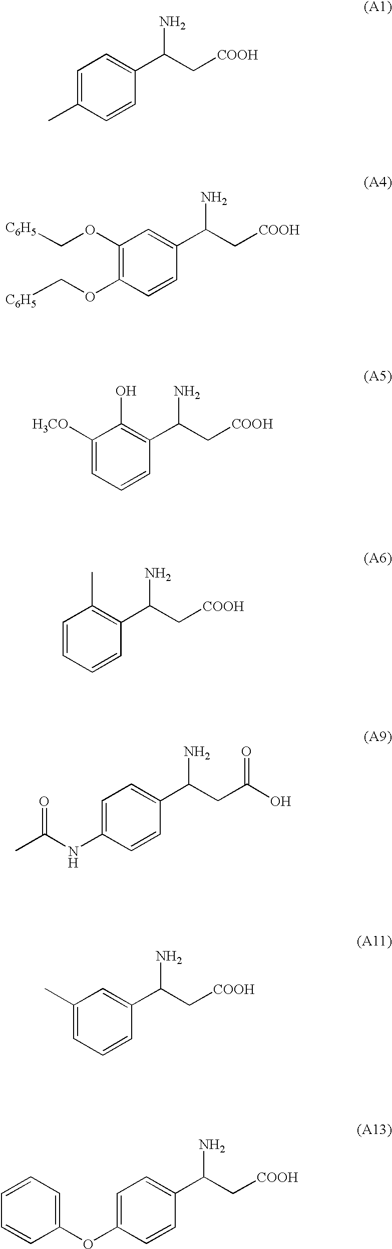

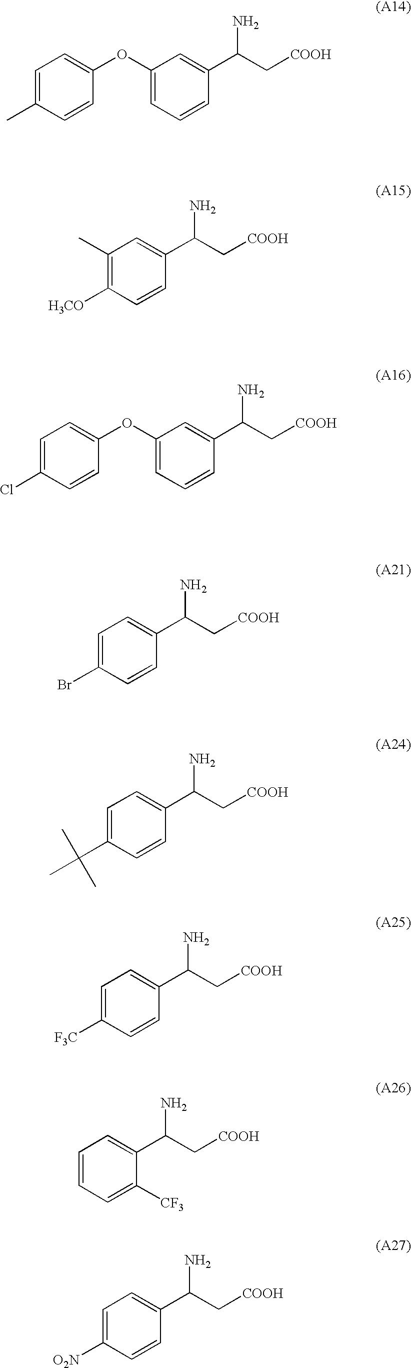

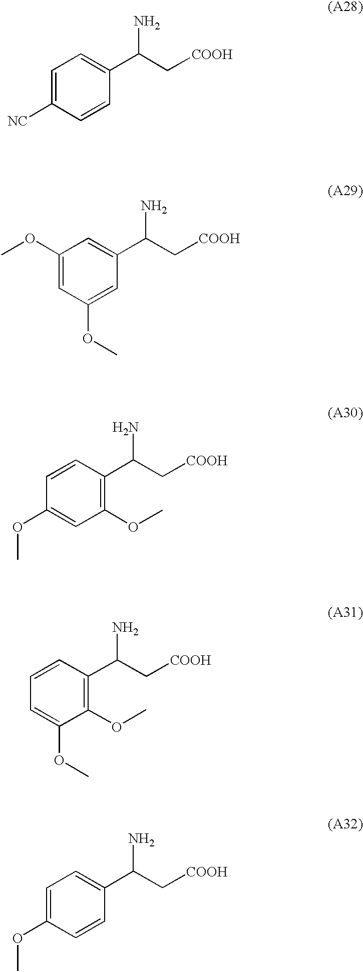

### Example 2

### In Vivo Assessment of Candidate Compounds' Pharmacological Utility for Inhibition of Epileptogenesis

The two groups of candidate analogues were tested in vivo for both anti-seizure activities and neurological toxicities. One seizure model was performed using adult male Sprague-Dawley rats in accordance with the guidelines of the Canada Council on Animal Care and under the supervision of the Queen's University Animal Ethics Committee. This test procedure has been adopted from previous work by Turski et al. (1984) Brain Res. 321:237. The test compounds were administered at 100 mg/kg by interperitoneal (i.p.) injection. Seizures were induced 20 minutes afterwards by i.p. administration of pilocarpine hydrochloride (350 mg/kg). Protection was defined as the absence of chronic spasms over a 30 minute observation period after pilocarpine administration. Compounds 1, 2, 3, 5, 8, 10, 11, 13, A1, A4, A5, A11, A13, A14, A15, A16, A21, A26, A28, A29, and A31 exhibited significant anti-seizure activity with this assay. The classes of compounds exhibiting anti-seizure activity include: N-substituted β-amino acid acid analogues (compounds 1, 2, 3, and 10); β-substituted β-amino acid analogues (compounds 5, 11, A1, A4, A5, A11, A13, A14, A15, A16, A21, A26, A28, A29, and A31); and α-substituted β-amino acid analogues (i.e. compounds 8 and 13).

Further assays to test the anti-seizure and neurotoxic properties of the candidate compounds included the maximal electroshock seizure (MES) model, the subcutaneous pentylenetetrazole (PTZ)-induced seizure model, and the rotorod neurotoxicity test. All assays were performed by the Anticonvulsant Drug Development (ADD) Program in the Epilepsy Branch of the NIH (see, e.g., Stables and Kupferberg (1997) The NIH anticonvulsant Drug Development (ADD) Program: Preclinical Anticonvulsant Screening Project, Libby & Sons). All compounds were tested with either male Carworth Farms #1 mice or male Sprague-Dawley rats. Each test compound was administered via an i.p. injection at 300, 100, and 30 mg/kg.

In the MES-induced seizure model, see, e.g., “Molecular and Cellular Targets for Anti-Epileptic Drugs” G. Avanzini, et al. (1997) John Libbey & Company Ltd., pp 191-198; Chapter 16, “The NIH Anticonvulsant Drug Development (ADD) Program: preclinical anticonvulsant screening project,” by James P. Stables and Harvey J. Kupferberg, anti-seizure activity of a test compound was defined as the abolition of hind-leg tonic-extension over a 30 minute observation period. Compounds 9, 10, and A3 showed significant anti seizure activity with this assay.

In the PTZ-induced seizure model, seizures were typically induced 0.5 and 4 hrs after test compound administration by i.p. injection of PTZ (85 mg/kg in mice and 70 mg/kg in rats). Protection was defined as the inhibition of chronic spasms over a 30 min observation period. Compounds 9, 10, A3, A7, A17, A22, A23, A24, and A25 showed significant anti seizure activity with this assay.

In the rotorod neurotoxicity testing, mice were placed on a 1 -inch diameter knurled plastic rod rotating at a speed of 6 rpm after the administration of the test compound. Neurotoxicity was defined as the inability of mice to maintain their equilibrium over a one minute observation period. Compounds 1, 2, 4-9, 11, 12, 14, A3, A4, A6, A8, A9, A10, A17, A21, A22, A23, A26, A27, A28, A29, A30, A31, and A32 showed no neurological toxicity by this assay. However, of the remaining compounds which exhibited some neurotoxicity, the level of toxicity was low compared to antiseizure drugs such as carbamazine and valproic acid.

### Example 3

### Synthesis of β-amino Acids:Method A

### General Procedures

### N-Acetyl Protection via Acetic Anhydride

Acetamidothiophenecarboxylic acid alkyl esters were prepared by refluxing the corresponding amino compound with excess Ac₂O (4 equiv.) in anhydrous AcOH for 1 hour. The mixture was poured in cold water and the product was isolated by filtration, washed with water and recrystallized from EtOH.

### Synthesis of Raney Nickel Catalyst

A solution of NaOH (320.0 g, 8 mol) in water (1.2 L) was mechanically stirred in a 2.0 L flask. After cooling to 10° C. in an ice-bath, nickel aluminum alloy (250 g) was added in small portions over 90 minutes. The resulting suspension was stirred at room temperature for 1 hour and at 50° C. for an additional 8 hours. The suspension was transferred to a graduated cylinder and the aqueous supernatant was decanted. The resulting slurry was shaken with 2.5 M aqueous NaOH solution (200 mL), then decanted. The nickel catalyst was washed 30 times by suspension in water (150 mL) followed by decanting. The washing was repeated 3 times with absolute EtOH (100 mL) and the resulting Raney nickel was stored under absolute EtOH.

### Raney Nickel Reductive Desulfurization

Alkyl acetamidothiophenecarboxylate (20 mmol) and freshly prepared Raney nickel (8 equiv.) were refluxed in EtOH (75 mL) with vigorous stirring for 16 hours. The hot mixture was filtered through diatomaceous earth (Celite) and the nickel residue was washed with hot EtOH (50 mL). The filtrate was concentrated to yield pure N-acetyl-β-alanine alkyl ester as a clear oil, a gum or white crystals.

### N-Acetyl and Alkyl Ester Deprotection via Acidolysis

The doubly protected α- or β-substituted β-alanine was refluxed in 6 M HCl for 5 hours. The solution was evaporated (to remove H₂O, HCl, MeOH and AcOH) and the residue was twice dissolved in distilled H₂O and concentrated (to remove residual HCl). The product was recrystallized from EtOH to yield the hydrochloride salt as white crystals. Alternatively, the crude product was dissolved in a minimum volume of hot H₂O and titrated with NH₄OH until the free β-amino acid precipitated. Two volumes of EtOH or MeOH were added to aid the separation of the product and prevent clumping. The mixture was cooled (4° C.) for 24 hours to encourage further precipitation then was filtered. The product was washed with ice cold H₂O and EtOH then was recrystallized from MeOH or EtOH to yield pure substituted β-alanine as white crystals.

### TLC Analysis

In the experimental procedures that follow, the solvents used for thin-layer chromatographic analysis are abbreviated as follows:

Solvent B: methylene chloride:acetone:acetic acid 100:100:0.5

Solvent I: ethyl acetate:methanol 9:1

Solvent J: chloroform:acetone:water 88:12:15

Solvent K: methanol:acetic acid 5:1

Solvent L: ethanol:acetic acid 50:1

### Synthesis of Alkyl Acetamidothiophenecarboxylates

### Methyl 3-Acelamidobenzo[b]thiophene-2-carboxylate

Using the procedure described above, methyl 3-aminobenzo[b]thiophene-2-carboxylate (1.8596 g, 8.97 mmol) was acetylated and purified by EtOH recrystallization to afford pure product as fine white crystals (1.4723 g, 5.91 mmol, 65.9%); mp: 178-180° C.; TLC: R_{f}=0.63 (Solvent I), 0.55 (Solvent J), 0.80 (Solvent L); IR (cm⁻¹): 3271 (NH), 3021 (CH), 1716 (ester C═O), 1670 (amide C═O), 746 (═CH); ¹H nmr (CDCl₃): δ9.46 (br s, 1H), 8.08 (dd, 1H, J=7.0, 2.2 Hz), 7.76 (dd, 1H, J=7.5, 1.0 Hz), 7.48 (d of t, 1H, J=6.9, 1.4 Hz), 7.39 (d of t, 1H, J=7.0, 1.0 Hz), 3.94 (s, 3H), 2.33 (s, 3H).

### Methyl 3-Acetamido-6-(trifluoromethyl)benzo[b]thiophene-2-carboxylate

Methyl 3-amino-6-(trifluoromethyl)benzo[b]thiophene-2-carboxylate (1.4944 g, 5.43 mmol) was acetylated and purified by EtOH recrystallization to afford pure product as fluffy, light yellow crystals (1.5261 g, 4.81 mmol, 88.6%); mp: 204-205° C.; TLC: R_{f}=0.72 (Solvent I), 0.78 (Solvent L); IR (cm⁻¹): 3274 (NH), 3069 (CH aromatic), 2962 (CH aliphatic), 1720 (ester C═O), 1676 (amino C═O); ¹H nmr (CDCl₃): δ9.81 (br s, 1H), 8.06 (s, 1H), 7.94 (d, 1H, J=8.7 Hz), 7.51 (dd, 1H, J=8.7, 1.4 Hz), 3.85 (s, 3H), 2.20 (d, 3H, J=4.2 Hz).

### Methyl 2-Acetamido-4,5,6,7-tetrahydrobenzo[b]thiophene-3-carboxylate

Methyl 2-amino-4,5,6,7-tetrahydrobenzo[b]thiophene-3-carboxylate (3.0004 g, 14.20 mmol) was acetylated as described above and purified by EtOH recrystallization to afford pure product as light brown crystals (3.3823 g, 13.35 mmol, 94.0%); mp: 103-106° C.; TLC: R_{f}=0.68 (Solvent I), 0.66 (Solvent J), 0.76 (Solvent L); IR (cm⁻¹): 3248 (NH), 2932 (CH), 1698 (ester C═O), 1668 (amide C═O); ¹H nmr (CDCl₃): δ11.22 (br s, 1H), 3.86 (s, 3H), 2.74 (m, 2H), 2.63(m, 2H), 2.25 (s, 3H), 1.79 (m, 2H), 1.76 (m, 2H).

### Methyl 2-Acetamido-6-tert-butyl-4,5,6,7-tetrahydrobenzo[b]thiophene-3-carboxylate

Methyl 2-amino-6-tert-butyl-4,5,6,7-tetrahydrobenzo[b]thiophene-3-carboxylate (1.3693 g, 5.12 mmol) was acetylated as described above and purified by EtOH recrystallization to afford pure product as fine white crystals (0.9312 g, 3.01 mmol, 58.8%); mp: 117-118° C.; TLC: R_{f}=0.74 (Solvent I), 0.70 (Solvent J); IR (cm⁻¹): 3271 (NH), 2953 (CH), 1674 (C═O); ¹H nmr (CDCl₃): δ11.20 (br s, 1H), 3.85 (s, 3H), 3.00 (d of m, 1H, J=17.1 Hz), 2.68 (d of m, 1H, J=15.7 Hz), 2.50 (d of m, 1H, J=17.3 Hz), 2.34 (d of m, 1H, J=14.2 Hz), 2.25 (s,3H), 2.00 (d of m, 1H, J=10.8 Hz), 1.49 (dd, 1H, J=12.0, 5.0 Hz), 1.27 (dd, 1H, J=12.1, 5.1 Hz), 0.93 (s, 9H).

### Ethyl 2-Acetamidocyclododeca[b]thiophene-3-carboxylate

Ethyl 2-aminocyclododeca[b]thiophene-3-carboxylate (4.9236 g, 15.91 mmol) was acetylated as described above and purified by EtOH recrystallization to afford pure product as light brown crystals (4.6058 g, 13.10 mmol, 82.3%); mp: 54-74° C.; TLC: R_{f}=0.73 (Solvent I), IR (cm⁻¹): 3358 (NH), 2929 (CH), 1710 (ester C═O), 1678 (amide C═O); ¹H nmr (CDCl₃): δ11.35 (br s, 1H), 4.33 (q, 2H, J=7.3 Hz), 2.75 (t, 2H, J=6.9 Hz), 2.69 (t, 2H, J=7.6 Hz), 2.47 (m, 2H), 2.44 (m, 2H), 2.24 (s, 3H), 1.74 (m, 4H), 1.62 (m, 4H), 1.38 (t, 3H, J=7.2 Hz), 1.30 (m, 4H).

### Methyl 2-Acetamido-4,5,6,7-tetrahydro-6-phenylbenzo[b]thiophene-3-carboxylate

Methyl 2-amino-4,5,6,7-tetrahydro-6-phenylbenzo[b]thiophene-3-carboxylate (2.5046 g, 8.71 mmol) was acetylated as described above and purified by EtOH recrystallization to afford pure product as a fine off-white powder (2.3763 g, 7.21 mmol, 82.8%); mp: 116-117° C.; TLC: R_{f}=0.79 (Solvent I), 0.78 (Solvent J); IR (cm⁻¹): 3255 (NH), 3029 (CH), 2925 (CH), 1686 (ester C═O), 1668 (amide C═O), 703 (═CH), ¹H nmr (CDCl₃): δ11.25 (br s, 1H), 7.28 (m, 5H), 3.88 (s, 3H), 3.00 (m, 2H), 2.89 (m, 2H), 2.78 (m, 1H), 2.27 (s, 3H), 2.08 (m, 1H), 1.94 (m, 1H).

### Methyl 3-Acetamido-5-phenylthiophene-2-carboxylate

Methyl 3-amino-5-phenylthiophene-2-carboxylate (2.5031 g, 10.73 mmol) was acetylated as described above and purified by EtOH recrystallization to afford pure product as white crystals (2.7726 g, 10.07 mmol, 93.8%); mp: 115° C.; TLC: R_{f}=0.70 (Solvent I), 0.70 (Solvent J); IR (cm⁻¹): 3319 (NH), 3122 (CH), 2950 (CH), 1715 (ester C═O), 1680 (amide C═O), 765 (═CH); ¹H nmr (CDCl₃): δ10.18 (br s, 1H), 8.38 (s, 1H), 7.66 (m, 2H), 7.41 (m, 3H), 3.90 (s, 3H), 2.25 (s, 3H).

### Methyl 3-Acetamido-5-(4-methoxyphenyl)thiophene-2-carboxylate

Methyl 3-amino-5-(4-methoxyphenyl)thiophene-2-carboxylate (2.5004 g, 9.50 mmol) was acetylated and purified by EtOH recrystallization to afford pure product as fine white crystals (2.7173 g, 8.90 mmol, 93.7%); mp: 148-149° C.; TLC: R_{f}=0.68 (Solvent I), 0.65 (Solvent J); IR (cm⁻¹): 3303 (NH), 3143 (CH), 2943 (CH), 1705 (ester C═O), 1663 (amide C═O), 817 (═CH); ¹H nmr (CDCl₃): δ10.19 (br s, 1H), 8.27 (s, 1H), 7.60 (d of m, 2H, J=8.9 Hz), 6.93 (d of m, 2H, J=8.8 Hz), 3.89 (s, 3H), 3.84(s,βH), 2.24(s, 3H).

### Methyl 3-Acetamido-5-(4-methylphenyl)thiophene-2-carboxylate

Methyl 3-amino-5-(4-methylphenyl)thiophene-2-carboxylate (1.5098 g, 6.10 mmol) was acetylated as described above and purified by EtOH recrystallization to afford pure product as white fluffy crystals (1.6694 g, 5.77 mmol, 94.6%); mp: 127-129° C.; TLC: R_{f}=0.70 (Solvent I), 0.64 (Solvent J), 0.75 (Solvent K); IR (cm ): 3316 (NH), 2953 (CH), 1710 (ester C═O), 1675 (amide C═O), 812 (═CH); ¹H nmr (CDCl₃): δ10.18 (br s, 1H), 8.33 (s, 1H), 7.56 (d, 2H, J=8.2 Hz), 7.21 (d, 2H, J=8.0 Hz), 3.89 (s, 3H), 2.38 (s, 3H), 2.24 (s, 3H).

### Methyl 3-Acetamido-5-[3-methoxy-4-(4-nitrobenzyloxy)phenylthiophene-2-carboxylate

Methyl 3-amino-5-[3-methoxy-4-(4-nitrobenzyloxy)phenyl]thiophene-2-carboxylate (1.5174 g, 3.66 mmol) was acetylated as described above and purified by EtOH recrystallization to afford pure product as yellow crystals (1.5487 g, 3.39 mmol, 92.6%); mp: 193-194° C.; TLC: R_{f}=0.68 (Solvent I), 0.65 (Solvent J); IR (cm⁻¹): 3326 (NH), 3072 (CH), 2944 (CH), 1705 (ester C═O), 1671 (amide C═O), 836 (═CH); ¹H nmr (CDCl₃): δ10.19 (br s, 1H), 8.28 (d, 2H, J=2 Hz), 8.23 (s, 1H), 7.62 (d, 2H, J=8.7 Hz), 7.19 (d, 2H, J=5.6 Hz), 6.85 (d, 1H, J=8.9), 5.27 (s, 2H), 3.97 (s, 3H), 3.90 (s, 3H), 2.24 (s, 3H).

### Synthesis of N-Acetyl-α-substituted-β-alanine Alkyl Esters

### N-Acetyl-α-cyclohexyl-β-alanine Methyl and Ethyl Esters

Methyl 2-acetamido-4,5,6,7-tetrahydrobenzo[b]thiophene-3-carboxylate (0.8125 g, 3.37 mmol) was reductively desulfurized using Raney nickel to yield the title compounds as a light yellow oil (0.6051 g, 2.81 mmol, 83.4%); TLC: R_{f}=0.80 (Solvent I), 0.81 (Solvent L); IR (cm⁻¹): 2894 (CH aliphatic), 1738 (ester C═O), 1674 (amide C═O); ¹H nmr (CDCl₃): δ5.91 (br s, 1H), 4.14 (q, 2H, J=7.1 Hz, minor ethyl ester product), 3.69 (s, 3H), 3.53 (m, 1H), 3.32 (m, 1H), 2.46 (m, 1H), 1.94 (s, 3H), 1.69 (m, 5H), 1.26 (t, 3H, J=7.2 Hz, minor ethyl ester product), 1.14 (m, 6H).

### N-Acetyl-α-cyclododecyl-β-alanine Ethyl Ester

Ethyl 2-acetamidocyclododeca[b]thiophene-3-carboxylate (2.3366 g, 6.65 mmol) was reductively desulfurized using Raney nickel to yield the title compound as a yellow oil (2.1314 g, 6.55 mmol, 98.5%); TLC: R_{f}=0.75 (Solvent I), 0.46 (Solvent J); IR (cm¹): 3316 (NH), 2903 (CH aliphatic), 1725 (ester C═O), 1661 (amide C═O); ¹H nmr (DMSO-d6): δ7.88 (br s, 1H), 4.05 (q, 2H, J=8.1 Hz), 3.59 (m, 2H), 2.45 (m, 1H), 1.74 (s, 3H), 1.50 (m, 1H), 1.28 (m, 22H), 1.15 (t, 3H, J=8.1 Hz).

### N-Acetyl-α-(4-tert-butylcyclohexyl)-β-alanine Methyl Ester

Methyl 2-acetamido-6-tert-butyl-4,5,6,7-tetrahydrobenzo[b]thiophene-3-carboxylate (0.8286 g, 2.68 mmol) was reductively desulfurized using Raney nickel to yield the title compound as a sticky white solid (0.7466 g, 2.63 mmol, 98.3%); mp: 73-75° C.; TLC: R_{f}=0.70 (Solvent I), 0.33 (Solvent J); IR (cm⁻¹): 3261 (NH), 2943 (CH aliphatic), 1735 (ester C═O), 1648 (amide C═O), ¹H nmr (CDCl₃): δ5.88 (br s, 1H), 3.69 (s, 3H), 3.53 (m, 1H), 3.41 (m, 1H), 3.34 (m, 1H), 2.44 (m, 1H), 1.94 (s, 3H), 1.77 (m, 2H), 1.63 (m, 1H), 1.50 (m, 1H), 1.27 (t, 1H, J=7.1 Hz), 1.00 (m, 4H), 0.82 (s, 9H).

### N-Acetyl-α-(4-phenylcyclohexyl)-β-alanine Methyl Ester

Methyl 2-acetamido-4,5,6,7-tetrahydro-6-phenylbenzo[b]thiophene-3-carboxylate (2.0292 g, 6.16 mmol) underwent Raney nickel reductive desulfurization to yield the title compound as a white solid (1.7908 g, 5.90 mmol, 95.8%); mp: 75-80° C.; TLC: R_{f}=0.58 (Solvent J), 0.79 (Solvent L); IR (cm⁻¹): 3259 (NH), 3079 (═CH), 2929 (CH aliphatic), 1730 (ester C═O), 1647 (amide C═O), 698 (═CH); ¹H nmr (CDCl₃): δ7.29 (m, 3H), 7.19 (m, 2H), 5.94 (br s, 1H), 3.73 (s, 3H), 3.58 (m, 1H), 3.48 (m, 1H), 3.40 (m, 1H), 2.47 (m, 2H), 1.97 (s, 3H), 1.91 (m, 2H), 1.75 (m, 2H), 1.50 (m, 2H), 1.26 (m, 2H).

### Synthesis of N-Acetyl-β-substituted-β-alanine Methyl Esters

### N-Acetyl-β-phenyl-β-alanine Methyl Ester

Methyl 3-acetamidobenzo[b]thiophene-2-carboxylate (1.3742 g, 5.51 mmol) underwent Raney nickel reductive desulfurization to yield the title compound as a light yellow-brown solid (1.1876 g, 5.37 mmol, 97.4%); mp: 58-61° C.; TLC: R_{f}=0.42 (Solvent I), 0.24 (Solvent J); IR (cm⁻¹): 3322 (NH), 3061 (CH aromatic), 2955 (CH aliphatic), 1741 (ester C═O), 1649 (amide C═O); ¹H nmr (CDCl₃): δ7.30 (m, 5H), 6.62 (br d, 1H, J=6.0 Hz), 5.43 (q, 1H, J=6.0 Hz), 3.62 (s, 3H), 2.89 (dd, 2H, J=8.5, 5.9Hz), 2.02 (s, 3H).

### N-Acetyl-β-(4-trifluoromethylphenyl)-β-alanine Methyl Ester

Methyl 3-acetamido-6-(trifluoromethyl)benzo[b]thiophene-2-carboxylate (0.7014 g, 2.21 mmol) was reductively desulfurized using Raney nickel to yield the title compound as a clear oil (0.5961 g, 2.05 mmol, 92.6%); TLC: R_{f}=0.52 (Solvent I), 0.86 (Solvent L); IR (cm⁻¹): 3340 (NH), 1736 (ester C═O), 1654 (amide C═O); ¹H nmr (DMSO-d6): δ8.45 (d, 1H, J=8.0 Hz), 7.59 (d, 2H, J=8.3 Hz), 7.49 (d, 2H, J=8.1 Hz), 5.25 (q, 1H, J=7.6, 15 Hz), 3.55 (s, 3H), 2.75 (m, 2H), 1.82 (s, 3H).

N-Acetyl-β-phenethyl-β-alanine Methyl Ester

Methyl 3-acetamido-5-phenylthiophene-2-carboxylate (2.3660 g, 8.59 mmol) underwent Raney nickel reductive desulfurization to yield the title compound as an off-white gum (2.1108 g, 8.47 mmol, 98.6%); TLC: R_{f}=0.68 (Solvent I), 0.65 (Solvent J); IR (cm⁻¹): 3475 (NH), 2893 (CH aliphatic), 1735 (ester C═O), 1654 (amide C═O); ¹H nmr (CDCl₃): δ7.23 (m, 5H), 6.10 (br d, 1H, J=8.8 Hz), 4.30 (t of d, 1H, J=8.9, 5.4 Hz), 3.68 (s, 3H), 2.66 (t, 2H, J=8.2 Hz), 2.57 (dd, 2H, J=4.9, 3.0 Hz), 1.96 (s, 3H), 1.87 (m, 2H).

### N-Acetyl-β-(p-methoxyphenethyl]-β-alanine Methyl Ester

Methyl 3-acetamido-5-(4-methoxyphenyl)thiophene-2-carboxylate (1.8100 g, 5.93 mmol) underwent Raney nickel reductive desulfurization to yield the title compound as a yellow oil (1.5544 g, 5.56 mmol, 93.8%); TLC: R_{f}=0.54 (Solvent I), 0.25 (Solvent J); IR (cm⁻¹): 3285 (NH), 2944 (CH), 1735 (ester C═O), 1651 (amide C═O), 728 (═CH); ¹H nmr (CDCl₃): δ7.08 (d, 2H, J=8.5 Hz), 6.81 (d, 2H, J=8.7 Hz), 6.03 (br d, 1H, J=8.7 Hz), 4.27 (m, 1H), 3.77 (s, 3H), 3.67 (s, 3H), 2.59 (t, 2H, J=8.2 Hz), 2.55 (d, 2H, J=8.4 Hz), 1.96 (s, 3H), 1.84 (q, 2H, J=8.2 Hz).

### N-Acetyl-β-[2-(4-methylphenyl)ethyl]-β-alanine Methyl Ester

Methyl 3-acetamido-5-(4-methylphenyl)thiophene-2-carboxylate (1.4905 g, 5.15 mmol) was reductively desulfurized using Raney nickel to yield the title compound as a white gum (1.3434 g, 5.10 mmol, 99.1%); mp: 50-51° C.; TLC: R_{f}=0.63 (Solvent I), 0.85 (Solvent L); IR (cm⁻¹): 3288 (NH), 2906 (CH aliphatic), 1731 (ester C═O), 1639 (amide C═O), 807 (═CH); ¹H nmr (CDCl₃): δ7.07 (s, 4H), 6.08 (br d, 1H, J=8.8 Hz), 4.28 (sextet, 1H, J=5.3 Hz), 3.67 (s, 3H), 2.63 (d, 2H, J=8.2 Hz), 2.55 (m, 2H), 2.30 (s, 3H), 1.96 (s, 3H), 1.84 (quintet, 2H, J=7.9 Hz).

### N-Acetyl-β-[2-(3-methoxy-4-hydroxyphenyl)ethyl]-β-alanine Methyl Ester

Methyl 3-acetamido-5-[3-methoxy-4-(4-nitrobenzyloxy)phenyl]thiophene-2-carboxylate (1.4481 g, 3.17 mmol) was reductively desulfurized using Raney nickel. The filtered solution was taken up in hot EtOAc then washed with 0.5 N HCl (2×30 mL) and H₂O. The organic layer was dried (MgSO₄), filtered and concentrated to yield the title compound as a yellow oil (0.5620 g, 1.90 mmol, 60.0%); TLC: R_{f}=0.80 (Solvent L); IR (cm⁻¹): 3498 (OH), 2905 (CH aliphatic), 1743 (ester C═O), 1663 (amide C═O), 726 (═CH); ¹H nmr (CDCl₃): δ6.82 (d, 1H, J=7.9 Hz), 6.67 (m, 2H), 6.10 (br d, 1H, J=8.6 Hz), 5.56 (br s, 1H), 4.28 (m, 1H), 3.88 (s, 3H), 3.68 (s, 3H), 2.60 (d, 2H, J=8.4 Hz), 2.55 (t, 2H, J=2.2 Hz), 1.97 (s, 3H), 1.85 (m, 2H).

### Synthesis of α-Substituted-β-alanines

### α-Cyclohexyl-β-alanine

N-Acetyl-α-cyclohexyl-β-alanine ethyl and methyl esters (2.4499 g, 10.77 mmol) were deprotected to yield the title compound as fine white crystals (0.9573 g, 5.59 mmol, 51.9%); mp: 238-240° C.; TLC: R_{f}=0.75 (Solvent I); IR (cm⁻¹): 3300-2700 (OH), 2207, 1635 (carboxylate C═O); ¹H nmr (TFA-d): δ4.58 (quintet, 2H), 4.01 (m, 1H), 3.11 (m, 1H), 2.83 (m, SH), 2.32 (m, SH).

### α-Cyclododecyl-β-alanine Hydrochloride Salt

N-Acetyl-α-cyclododecyl-β-alanine ethyl ester (2.1268 g, 6.83 mmol) was deprotected to yield the title compound as white crystals (0.7322 g, 2.51 mmol, 36.7%); mp: 201-204° C.; TLC: R_{f}=0.79 (Solvent I), 0.80 (Solvent L); IR (cm⁻¹): 3400-2700 (OH), 1722 (carboxylate C═O); ¹H nmr (DMSO-d6): δ12.72 (br s, 1H), 7.99 (br s, 3H), 2.98 (m, 1H), 2.82 (m, 1H), 2.68 (m, 1H), 1.91 (m, 2H), 1.28 (m, 22H).

### α-(4-tert-Butylcyclohexyl)-β-alanine Hydrochloride Salt

N-Acetyl-α-(4-tert-butylcyclohexyl)-β-alanine methyl ester (0.7463 g, 2.63 mmol) was deprotected to yield the title compound as fine white crystals (0.4347 g, 1.65 mmol, 62.7%); mp: 230° C. (dec); TLC: R_{f}=0.91 (Solvent K); IR (cm⁻¹): 3400-2700 (OH), 1732 (carboxylate C═O); ¹H nmr (DMSO-d6): δ8.02 (br s, 3H), 2.97 (m, 1H), 2.84 (m, 2H), 2.51 (m, 1H), 1.71 (m, 3H), 1.63 (m, 2H), 0.95 (m, 4H), 0.79 (s, 9H).

### α-(4-Phenylcyclohexyl)-β-alanine Hydrochloride Salt

N-Acetyl-α-(4-phenylcyclohexyl)-β-alanine methyl ester (1.6699 g, 5.50 mmol) was deprotected to yield the title compound as fine white crystals (0.5235 g, 1.84 mmol, 33.5%); mp: 268° C. (dec); TLC: R_{f}=0.74 (Solvent I), 0.64 (Solvent K); IR (cm⁻¹): 3300-2500 (OH), 1701 (carboxylate C═O); ¹H nmr (DMSO-d6): δ8.09 (br s, 0.5H), 7.18 (m, 5H), 3.29 (m, 1H), 3.01 (m, 1H), 2.87 (dd, 1H, J=12.8, 4.0 Hz), 2.57 (t, 1H, J=4.5 Hz), 2.45 (m, 1H), 1.75 (m, 5H), 1.29 (m, 3H).

### Synthesis of β-Substituted-β-Alanines

### β-Phenyl-β-alanine

N-Acetyl-β-phenyl-β-alanine methyl ester (1.1561 g, 5.23 mmol) was deprotected to yield the title compound as fine white crystals (0.5275 g, 3.19 mmol, 61.1%); mp: 220-221° C.; TLC: R_{f}=0.75 (Solvent I); IR (cm⁻¹): 3305 (sharp: OH not H-bonded), 2195, 1627 (carboxylate C═O); ¹H nmr (D₂O): δ7.32 (s, 5H), 4.49 (t, 1H, J=7.9 Hz), 2.71 (d of t, 2H, J=6.5, 1.3 Hz).

### β-(4-Trifluoromethylphenyl)-β-alanine Hydrochloride Salt

N-Acetyl-β-(4-trifluoromethylphenyl)-β-alanine methyl ester (0.5850 g, 2.01 mmol) was deprotected to yield the title compound as a white powder (0.5076 g, 1.87 mmol, 93.0%); mp: 203° C. (dec.); TLC: R_{f}=0.60 (Solvent H); IR (cm⁻¹): 3500-2900 (OH), 1715 (carboxylate C═O); ¹H nmr (D₂O): δ7.70 (d, 1H, J=8.1 Hz), 7.54 (d, 2H, J=8.1 Hz), 4.78 (dd, 1H, J=7.0, 7.3 Hz), 3.05 (m, 2H).

### β-Phenethyl-β-alanine

N-Acetyl-β-2-phenethyl-β-alanine methyl ester (1.5322 g, 6.15 mmol) was deprotected to yield the title compound as white crystals (0.4709 g, 2.44 mmol, 39.6%); mp: 211-214° C.; TLC: R_{f}=0.37 (Solvent I), 0.74 (Solvent L); IR (cm⁻¹): 3496, 3310 (sharp: OH not H-bonded), 3028 (CH), 2932 (CH), 2162, 1663 (carboxylate C═O), 702 (═CH); ¹H nmr (TFA-d): δ8.36 (d, 5H, J=15.6 Hz), 4.92 (br s, 1H), 4.14 (br s, 2H), 3.95 (br d, 2H, J=8.0 Hz), 3.32 (br s, 2H).

### β-(p-Methoxyphenethyl)-β-alanine

N-Acetyl-β-(p-methoxyphenethyl)-β-alanine methyl ester (1.1244 g, 4.03 mmol) was deprotected and recrystallized from MeOH to give the title compound as off-white crystals (0.2761 g, 1.25 mmol, 31.0%); mp: 180-184° C.; TLC: R_{f}=0.34 (Solvent I), 0.70 (Solvent K); IR (cm⁻¹): 3400-2500 (OH), 2171, 1632 (carboxylate C═O); ¹H nmr (D₂O): δ7.13 (d, 2H, J=8.6 Hz), 6.85 (d, 2H, J=8.5 Hz), 3.69 (s, 3H), 3.37 (m, 1H), 2.57 (t, 2H, J=8.0 Hz), 2.46 (m, 2H), 1.82 (m, 2H).

### β-(p-Methylphenethyl)-βalanine

N-Acetyl-β-[2-(4-methylphenyl)ethyl]-β-alanine methyl ester (1.2884 g, 4.89 mmol) was deprotected to yield the title compound as fluffy white crystals (0.6779 g, 3.27 mmol, 66.9%); mp: 206-207° C.; TLC: R_{f}=0.89 (Solvent K); IR (cm⁻¹): 3530, 3280 (sharp: OH not H-bonded), 3017 (CH), 2166, 1706 (carboxylate C═O), 810 (═CH); ¹H nmr (TFA-d): δ8.20 (m, 4H), 4.89 (m, 1H), 4.10 (m, 2H), 3.87 (m, 2H), 3.38 (s, 3H), 3.28 (quintet, 2H, J=6.32 Hz).

### β-[2-(4-Hydroxy-3-methoxyphenyl)ethyl]-β-alanine Hydrochloride Salt

N-Acetyl-β-[2-(4-hydroxy-3-methoxyphenyl)ethyl]-β-alanine methyl ester (0.5281 g, 1.79 mmol) was deprotected to yield the title compound as a yellow oil (0.4852 g, 1.76 mmol, 98.4%); TLC: R_{f}=0.32 (Solvent I), IR (cm⁻¹): 3447 (OH), 1718 (carboxylate C═O); ¹H nmr (DMSO-d6): 7.79 (br d, 1H, J=8.3 Hz), 6.68 (s, 1H), 6.65 (d, 1H, J=9.5 Hz), 6.49 (d, 1H, J=8.0 Hz), 4.00 (m, 1H), 3.69 (s, 3H), 2.43 (m, 2H), 2.30 (d, 2H, J=6.6 Hz), 1.63 (m, 2H).

### Synthesis of 2-Azetidinones

### Preparation of N-Substituted 2-Azetidinones from N-Substituted β-Amino Acids

CCl₄ (1.0 mL, 10 mmol) and triethylamine (TEA) (1.7 mL, 12 mmol) were added to a stirred solution of N-substituted β-amino acid (10 mmol) and (C₆H₅)₃P (1.56 g, 1.2 mmol) in MeCN (100 mL). The reaction mixture was refluxed for 1.5 hours then concentrated in vacuo. The residue was dissolved in CH₂Cl₂ (100 mL) and washed with water and brine. The organic layer was dried (MgSO₄) and evaporated to dryness. The product was isolated by silica gel flash chromatography using EtOAc/hexane (1:2) as an eluant.

### Preparation of N-Silyl 2-Azetidinones from N-Unsubstituted β-Amino Acids

N-Bromosuccinimide (2.14 g, 12 mmol) and TEA (1.7 mL, 12 mmol) were added to a stirred solution of N-unsubstituted β-amino acid (10 mmol) and (C₆H₅)₃P (1.56 g, 1.2 mmol) in MeCN (100 mL). The reaction mixture was stirred at ambient temperature for 10 hours, then concentrated in vacuo. The residue was dissolved in CH₂Cl₂ (60 mL), treated with t-butyldimethylsilyl chloride (2.25 g, 15 mmol) and diisopropylamine (2.8 mL, 15 mmol), and stirred at room temperature for 5 hours. The solution was then diluted with CH₂Cl₂ (100 mL) and washed with water and brine. The organic layer was dried (MgSO₄) and evaporated to dryness. The product was isolated by silica gel flash chromatography using EtOAc/hexane (1:7) as an eluant.

### Example 4

### Synthesis of β-aryl β-alanines

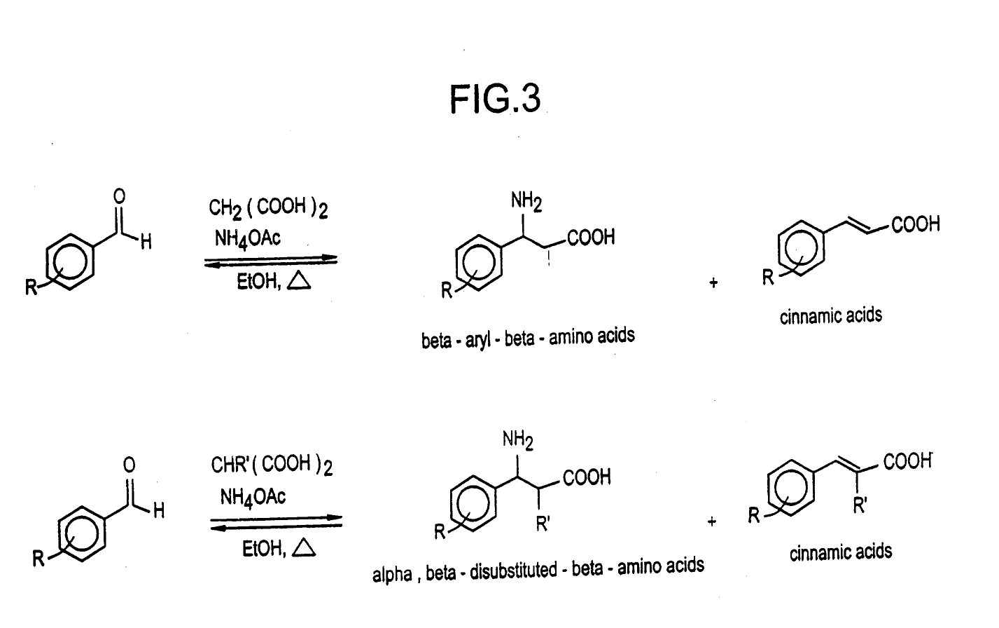

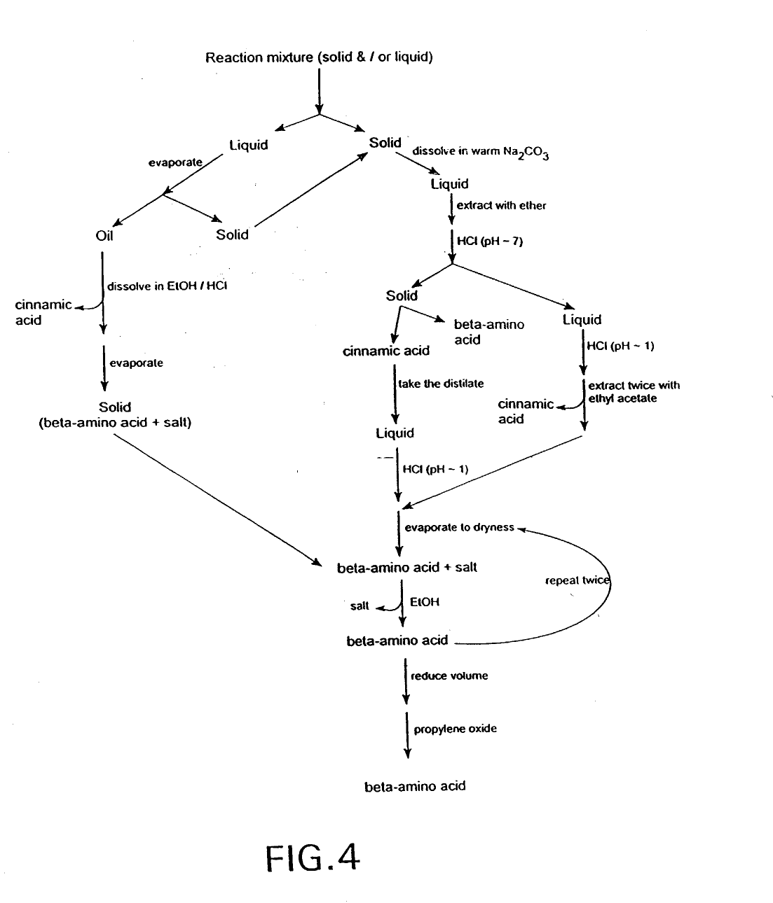

β-Aryl-β-alanines were prepared in a one-pot reaction. In brief, to a solution of a substituted benzaldehyde in absolute ethanol was added malonic acid and excess ammonium acetate, and the reaction mixture was heated to reflux. The reaction mixture was cooled to yield a mixture of the β-aryl-β-alanine and (in certain cases) a cinnamic acid derivative. The cinnamic acid (if present) was removed by acid/base extraction of the mixture to yield the β-aryl-β-alanine, often in moderate to good yield. The process is depicted in FIG. 3, and further details of experimental procedures for the synthesis of certain β-aryl-β-alanine compounds are provided infra. A representative purification scheme for purifying the compounds is shown in FIG. 4. Certain compounds prepared as described herein are set forth in Table 1, infra. Yield data are presented in two columns, the second being identical to that in Table 2, infra.

TABLE 1
Average yield of β-aryl-β-alanines prepared from benzaldehydes
(Reaction conditions not optimized)

| Compound RCH(NH₂)CH₂COOH | Average Yield (%) |
|---|---|
| R = | |
| 4-Fluorophenyl | 65% |
| 4-Phenoxyphenyl | 54% |
| 3-Methylphenyl | 56% |
| 3-Methyl-4-methoxyphenyl | 53% |
| 3-(3,4-dichlorophenoxy)phenyl | 49% |
| 2-Methylphenyl | 19% |
| 3-(4-chlorophenoxy)phenyl | 28% |
| 2,5-Dimethyl-4-methoxyphenyl | 18% |
| 4-Trifluoromethoxyphenyl | 31% |
| 2-Chlorophenyl | 25% |
| 2-Fluoro-3-trifluoromethylphenyl | 11% |
| 3-Bromo-4-methoxyphenyl | 34% |
| 4-Bromophenyl | 52% |
| Phenyl | 64% |
| 4-Methylphenyl | 51% |
| 4-Chlorophenyl | 39% |
| 4-Acetamidophenyl | 23% |
| 2,5-Dimethoxyphenyl | 22% |
| 4-Diethylaminophenyl | |
| 3-Methylphenyl | 46% |
| 2-Hydroxy-3-methoxyphenyl | 14% |
| 4-Phenylphenyl | 40% |
| 3,4-Dibenzyloxyphenyl | 36% |
| 3-[(3-Trifluoromethyl)phenyloxy]phenyl | 35% |

Selected compounds synthesized by this method are shown in Table 1. Representative syntheses of certain of these compounds, and additional compounds of the invention, are set forth below.

β-substituted-β-amino-acids were prepared by refluxing the corresponding benzaldehyde derivatives with excess ammonium acetate (˜2 equiv.), and malonic acid (1 equiv.) in absolute ethanol until the reaction has completed (determined by TLC and NMR). Cinnamic acid derivative was produced as a side product. The reaction mixtures were then worked up with standard procedures, e.g., as described in FIG. 4.

### β-3(3,4-dichlorophenoxy)phenyl-β-alanine Hydrochloride Salt

Using the procedure described above, 3-(3,4-dichlorophenoxy)benzaldehyde (10 g, 37.4 mmol), ammonium acetate (3.8437 g, 49.8 mmol) and malonic acid (3.8923 g, 37.4 mmol) were refluxed (slow) in absolute ethanol (30 mL) for 5 hours. β-3(3,4-dichlorophenoxy)phenyl-β-alanine as white solid was then filtered and washed twice with 10 mL of absolute ethanol. Subsequently, addition of 10 mL 3N HCl was added to this β-3(3,4-dichlorophenoxy)phenyl-β-alanine to afford the β-3(3,4-dichlorophenoxy)phenyl-β-alanine hydrochloride salt (4.44 g, 12.2 mmol, 32.6%); MP: 164-165° C.; IR (KBr): 3193, 1609 cm⁻¹; R_{f}=0.55 (solvent 24), 0.72 (solvent 25); ¹H NMR (D₂O/K₂CO₃): δ7.31-6.57 (m, 7H), 4.03 (t, J=7.29 Hz, 1H), 2.4-2.29 (m, 2H). Anal. Calcd for C₁₅H₁₄Cl₃NO₃: C, 49.68; H, 3.89; N, 3.86. Found: C, 49.34; H, 3.87; N, 3.93.

### β-4-bromophenyl-β-alanine

4-Bromobenzaldehyde (10 g, 54 mmol), ammonium acetate (8.663 g, 112.4 mmol) and malonic acid (5.6762 g, 54.5 mmol) were refluxed (slow) in absolute ethanol (45 mL) for 150 hours. White solid was filtered and dissolved into a warm (70° C.) solution of 50 mL of Na₂CO₃ and 50 mL of H₂O. This solution was then extracted with 100 mL of diethyl ether three times. The aqueous layer was further acidified to pH 7 to produce white solid β-4-bromophenyl-β-alanine (4.5140 g, 18.49 mmol, 34.2%); MP: 234° C.; IR (KBr): 3061, 1594 cm⁻¹; TLC: R_{f}=0.35 (solvent 24), 0.32 (solvent 25); ¹H NMR (D₂O/K₂CO₃): δ7.42-7.38 (m, 2H), 7.17-7.14 (m, 2H), 4.11-4.07 (t, J=7.25 Hz, 1H), 2.48-2.36 (m, 2H). Anal. Calcd for C₉H₁₀BrNO₂: C, 44.29; H, 4.13; N, 5.74. Found: C, 44.35; H, 3.93; N, 5.70.

### β-4-fluorophenyl-β-alanine

4-Fluorobenzaldehyde (10 g, 80 mmol), ammonium acetate (8.2487 g, 107 mmol) and malonic acid (8.3285 g, 80 mmol) were refluxed (slow) in absolute ethanol (60 mL) for 48 hours. White solid was filtered and purified by ethanol recrystallization to afford β-4-fluorophenyl-β-alanine (10.04 g, 54.8 mmol, 68.5%); MP: 216-217° C.; IR (KBr): 3160, 1606 cm⁻¹; TLC: R_{f}=0.41 (solvent 24), 0.42 (solvent 25); ¹H NMR (D₂O/K₂CO₃): δ7.28-7.19 (m, 2H), 7.03-6.91 (m, 2H), 4.10 (t, J=7.39 Hz, 1H), 2.54-2.34 (m, 2H). Anal. Calcd for C₉H₁₀FNO₂.5/3H₂O: C, 50.70; H, 6.30; N, 6.57. Found: C, 50.34; H, 6.39; N, 6.30.

### β-2,5-dimethoxyphenyl-β-alanine

2,5-dimethoxybenzaldehyde (4.1437 g, 25 mmol), ammonium acetate (3.1200 g, 40.47 mmol) and malonic acid (3.1244 g, 30.02 mmol) were refluxed (slow) in absolute ethanol (60 mL) for 6 hours. White solid was filtered and purified by methanol recrystallization to afford β-2,5-dimethoxyphenyl-β-alanine (1.239 g, 5.5 mmol, 22.0%); MP: 206-208° C.; IR (KBr): 2944, 1630 cm⁻¹; TLC: R_{f}=0.29 (solvent 21), 0.66 (solvent 23); ¹H NMR (200 MHz, D₂O/K₂CO₃): δ6.9-6.7 (m, 3H), 4.3 (t, J=7.89 Hz, 1H), 3.7-3.6 (m, 6H) 2.55-2.2 (m, 2H). Anal. Calcd for C₁₁H₁₅NO₄.6/5H₂O: C, 53.52; H, 7.10; N, 5.67. Found: C, 53.85; H, 6.45; N, 5.56.

### β-3-bromo-4-methoxyphenyl-β-alanine

3-Bromo-4-methoxylbenzaldehyde (9.9835 g, 46.42 mmol), ammonium acetate (7.2984 g, 94.69 mmol) and malonic acid (4.9124 g, 47.21 mmol) were refluxed (slow) in absolute ethanol (110 mL) for 281 hours. White solid was filtered and dissolved into a warm (70° C.) solution of 50 mL of Na₂CO₃ and 50 mL of H₂O. This solution was then extracted with 100 mL of diethyl ether three times. The aqueous layer was further acidified to pH 1 and extracted with 100 mL of ethyl acetate twice. Subsequently the aqueous layer was evaporated to dryness and 30 mL of absolute ethanol was then added to the white residue, stirred for 15 min, and filtered. The same procedure was then repeated twice. The final mixture was filtered, and the filtrate was evaporated to dryness. Propylene oxide (9.75 mL, 139.3 mmol) was added to the ethanol portion. The solution was stirred and warmed up to 50° C. to produce β-3-bromo-4-methoxyphenyl-β-alanine (3.0284 g, 11.05 mmol, 23.8%); MP: 213° C.; IR (KBr): 2945, 1604 cm⁻¹; TLC: R_{f}=0.26 (solvent 24), 0.28 (solvent 25); ¹H nmr (D₂O/K₂CO₃): δ7.42 (s, 1H), 7.18-7.14 (d d, 1H), 6.91-6.87 (d, 1H), 4.05-3.98 (t, 1H), 3.71 (s, 1H), 2.47-2.30 (m, 2H). Anal. Calcd for C₁₀H₁₂BrNO₃1/5H₂O: C, 43.25; H, 4.50; N, 5.04. Found: C, 43.16; H, 4.24; N, 4.94.

Additional compounds as synthesized generally in accordance with the previous paragraphs and analytical data therefor are provided below in Table 2.

TABLE 2
β-aryl-β-alanines prepared from benzaldehydes.

| | | m.p. | TLC | |
|---|---|---|---|---|
| Compound | Yield | (° C.) | (R_{f}) | NMR (PPM) |
| B5P91 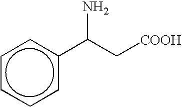 C₉H₁₁NO₂ MW = 165.20 | 67.1% | 220-221 | 21: 0.54 23: 0.60 | 7.35-7.2 (s, 5H) 4.45 (t, 1H, 7.3 Hz) 2.8-2.1 (m, 2H) solubility: ˜10 mg/ml saline |
| B6P165 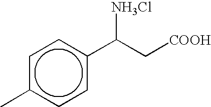 C₁₀H₁₄NO₂Cl MW = 215.68 | 51% | 208-210 | 21: 0.57 23: 0.56 | 7.2-7.1 (M, 4H) 4.17-4.09 (t, 1H, 7.4 Hz) 2.39-2.46 (m, 2H) solubility: ˜10 mg/ml saline |
| B6P169 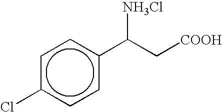 C₉H₁₁NO₂Cl₂ MW = 236.10 | 65% | 186-189 | 21: 0.54 23: 0.54 | 7.3-7.17 (s, 4H) 4.07-4.17 (t, 7H, 7.2 Hz) 2.45-2.55 (dt, 4.5 Hz, 3.5 Hz) solubility: ˜10 mg/ml saline |
| B7P16 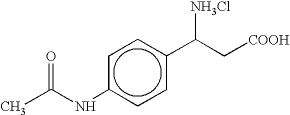 C₁₁H₁₄N₂O₃ MW = 222.24 | 23% | 221-222 | 21: 0.32 23: 0.60 | 7.2-7.3 (s, 4H) 4.05-4.15 (t, 1H, 7.4 Hz) 2.4-2.5 (dt, 4.9 Hz, 2.5 Hz) solubility: ˜10 mg/ml saline |
| B8P22 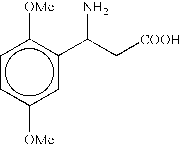 C₁₁H₁₅NO₄MW = 225.23 | 22% | 206-208 | 21: 0.29 23: 0.66 | 6.9-6.7 (m, 3H) 4.3 (t, 1H, 7.89 Hz) 3.7-3.6 (m, 6H) 2.55-2.2 (m, 2H) |
| B8P25 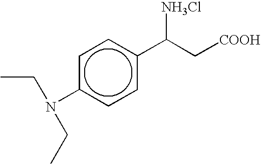 C₁₃H₂₁N₂O₂Cl MW = 272.77 | | 228 | 21: 0.298 23: 0.48 24: 0.48 | 6.7-6.8 (d, 2H, 8.71 Hz) 7.1-7.2 (d, 2H, 8.72 Hz) 4.0-4.1 (t, 1H, 7.28 Hz) 3.0-3.1 (M, 4H) 2.3-2.4 (M, 2H) 0.8-0.9 (M, 6H) |
| B8P58 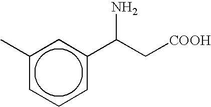 C₁₀H₁₃NO₂MW = 179.22 | 45.8% | 226-227 | 24: 0.297 25: 0.324 | 6.9-7.2 (M, 4H) 4.0-4.1 (t, 1H, 7.37 Hz) 2.4 (M, 2H) 2.2 (M, 3H) |
| B8P13 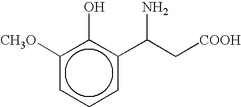 C₁₀H₁₃NO₄MW = 211.22 | 17.2% | 200-201 | 24: 0.324 25: 0.324 | 6.6-6.8 (M, 3H) 4.4-4.5 (t, 1H, 7.30 Hz) 3.6 (s, 3H) 2.5 (dd, 2H, 7.25 Hz) |
| B8P85 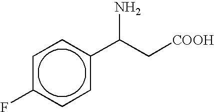 C₉H₁₀FNO₂MW = 183.17 | 61.5% | 216-217 | 24: 0.41 25: 0.42 | 7.28-7.19 (m, 2H) 7.03-6.91 (m, 2H) 4.10 (t, 1H, 7.39 Hz) 2.54-2.34 (m, 2H) |
| B8P79 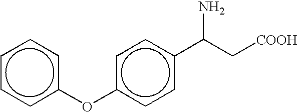 C₁₅H₁₅NO₃MW = 257.29 | 68.1% | 214-215 | 24: 0.65 25: 0.43 | 7.33-7.23 (m, }7.09-7.03 (m, } 9H 6.96-6.89(m, }4.08-4.16 (t, 1H, 7.23 Hz) 2.46-2.42 (dd, 2H, 7.12 Hz, 2.386 Hz) |
| B8P91 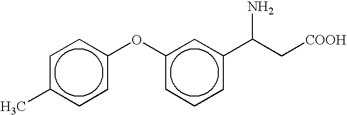 C₁₆H₁₇NO₃MW = 271.32 | 56.4% | 205-208 | 24: 0.53 25: 0.58 | 7.28-6.77 (m, 8H) 4.08 (t, 1H, 7.30 Hz) 2.42-2.38 (d, 2H, 7.29 Hz) 2.189 (s, 3H) |
| B8P89 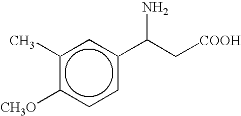 C₁₁H₁₅NO₃MW = 209.31 | 52.7% | 237-240 | 24: 0.22 25: 0.46 | 7.07-7.1 (m, 2H) 6.82-6.88 (m, 1H) 4.05-4.12 (t, 1H, 7.286 Hz) 3.708 (s, 3H) 2.39-2.46 (m, 2H) 2.064 (s, 3H) |
| B8P81 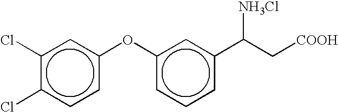 C₁₅H₁₄Cl₃NO₃MW = 364.14 | 42.6% | 164-165 | 24: 0.55 25: 0.72 | 7.31-6.57 (m, 7H) 4.03 (t, 1H, 6.38 Hz) 2.4-2.29 (m, 2H) |
| B8P74 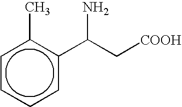 C₁₀H₁₃NO₂MW = 179.22 | 19.0% | 219 | 24: 0.487 25: 0.308 | 7.30-7.27 (m, 1H) 7.20-7.05 (m, 3H) 4.1-4.0 (t, 1H, 7.35 Hz) 2.44-2.39 (dd, 2H, 6.56 Hz, 1.93 Hz) 2.26-2.24 (s, 3H) |
| B8P95 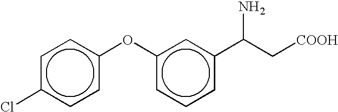 C₁₅H₁₄ClNO₃MW = 291.73 | 33.2% | 202-203 | 24: 0.52 25: 0.488 | 7.29-7.22 (m, }7.06-7.03 (d, } 8H 6.91-6.81 (m, }4.08 (t, 1H, 7.29 Hz) 2.42-2.38 (d, 1H, 7.25 Hz) |
| B8P93 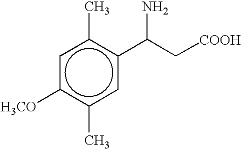 C₁₂H₁₇NO₃MW = 223.27 | 22.6% | 228 | 24: 0.58 25: 0.62 | 7.07 (s, 1H) 6.71 (s, 1H) 4.38 (t, 1H, 6.89 Hz) 3.69 (s, 3H) 2.39-2.36 (d, 2H, 7.24 Hz) 2.20 (s, 3H) 2.03 (s, 3H) |
| B8P101 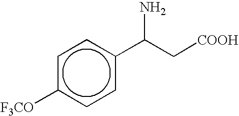 C₁₀H₁₀F₃NO₃MW = 249.19 | 46.2% | 222-223 | 24: 0.64 25: 0.268 | 7.34-7.30 (d, 2H, 8.71 Hz) 7.20-7.16 (d, 2H, 8.102 Hz) 4.18-4.11 (1, 1H, 7.23 Hz) 2.46-2.41 (dd, 2h, 7.426 Hz, 2.914 Hz) |
| B8P68 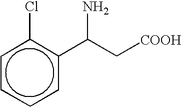 C₉H₁₀ClNO₂MW = 199.64 | 27.7% | 219 | 24: 0.38 25: 0.61 | 7.38-7.12 (m, 4H) 5.05 (t, 1H, 6.4 Hz) 2.62-2.27 (m, 2H) |
| B8P83 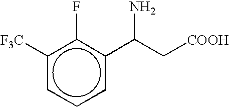 C₁₀H₉F₄NO₂MW = 251.18 | 15.5% | 206 | 24: 0.486 25: 0359 | 7.54-7.50 (m, 2H) 7.24-7.20 (t, 1H, 7.912 Hz) 4.50-4.37 (t, 1H, 7.3 Hz) 2.53-2.49 (d, 2H, 7.38 Hz) |
| B8P135 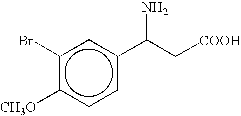 C₁₀H₁₂BrNO₃MW = 274.11 | 43.8% | 213 | 24: 0.256 25: 0.275 | 7.42 (s, 1H) 7.18-7.14 (d of d, 1H) 6.87-6.91 (d, 1H) 4.05-3.98 (t, 2H) 3.71 (s, 3H) 2.47-2.30 (m, 2H) |
| B8P163 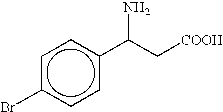 C₉H₁₀BrNO₂MW = 244.09 | 69.2% | 234 | 24: 0.35 25: 0.32 | 7.38-7.42 (m, 2H) 7.14-7.17 (m, 2H) 4.07-4.11 (t, 1H, 7.25 Hz) 2.36-2.48 (m, 2H) |
| B8P159 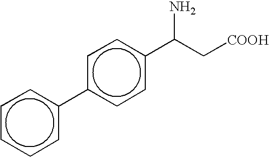 C₁₅H₁₅NO₂MW = 241.29 | 40.2 | 244 | 24: 0.27 25: 0.47 | 7.19-7.46 (m, 9H) 4.13-4.18 (t, 1H, 6.7 Hz) 2.39-2.43 (d, 2H, 7.2 Hz) |
| B8P147 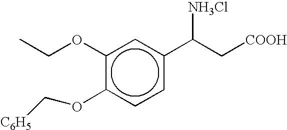 C₂₃H₂₄ClNO₄MW = 413.90 | 36.2 | 198-200 | 24: 0.41 25: 0.43 | 7.35-7.21 (m, 10H) 7.07-6.92 (m, 3H) 5.07 (s, 4H) 4.41-4.37 (t, 1H, 8.86) 2.89-2.83 (m, 2H) |
| B8P155 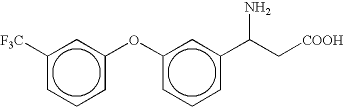 C₁₆H₁₄F₃NO₃MW = 413.90 | 39.7 | 192-194 | 24: 0.49 25: 0.44 | 7.53-7.37 (m, 3H) 7.23-7.13 (m, 4H) 7.02-6.97 (m, 1H) 4.49-4.45 (1, 1H, 7.1 Hz) 2.64-2.61 (m, 2H) |

### TLC Analysis

In the experimental procedures above, the solvents used for thin layer chromatographic analysis are abbreviated as follow:

Solvent 21: acetonitrile:acetic acid:water 8:1:1

Solvent 23: methanol:acetic acid 7:1

Solvent 24: n-butanol:acetic acid: water 4:1:1

Solvent 25: methanol:chloroform:acetic acid 7:7:1:

Additional analytical and biological data for β-aryl-β-alanines, β-phenethyl-β-alanines, α-cyclohexyl-β-alanines, and α-substituted-β-alanines (and certain esters and amides thereof) as well as 4′-substituted N-acetyl-α-piperidinyl-β-alanine, are shown in Tables 3-1 to 3-3.

TABLE 3-1
Analytical and Biological Activity Data
A. β-Aryl-β-Alanines and Precursors

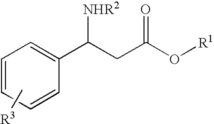

| | | | | Yield^{a} | Biological |
|---|---|---|---|---|---|
| Compound | R¹ | R² | R³ | (%) | Activity^{b} |
| B5P65 | CH₃ | Ac | H | 97.4 | NA |
| B6P140 | CH₃ | Ac | ρ-F₃C | 87.1 | NA |
| B5P91 | H | H | H | 61.1 | Inactive-0 |
| B6P141 | H | H.HCl | ρ-F₃C | 93.0 | Active-+1 |

B. Aryl Substituted β-Phenethyl-β-Alanine and Precursors

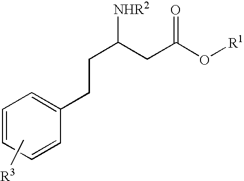

| | | | | Yield^{a} | Biological |
|---|---|---|---|---|---|
| Compound | R¹ | R² | R³ | (%) | Activity^{b} |
| B5P69 | CH₃ | Ac | ρ-CH₃O | 93.8 | NA |
| B5P73 | CH₃ | Ac | H | 98.6 | NA |
| B6P89 | CH₃ | Ac | ρ-CH₃ | 99.1 | NA |
| B6P101 | CH₃ | Ac | m-NEt | 100 | NA |
| B6P113 | CH₃ | Ac | m,ρ- | 97.5 | NA |
| | | | OCH₂O— | | |
| B6P119 | CH₃ | Ac | ρ-OH | 60.0 | NA |
| | | | m-CH₃O | | |
| B5P81 | H | H | ρ-CH₃O | 31.0 | Inactive - 0 |
| B5P95 | H | H | H | 39.6 | Active - +1 |
| B5P111 | H | H | ρ-CH₃ | 66.9 | Inactive - 0 |
| B6P145 | H | H | ρ-OH | 98.4 | Active - +1 |
| | | | m-CH₃O | | |

^{a}EtOH, H₂O or a mix used for recrystallization, where possible;
^{b}Using pilocarpine, compound is active in rat at 100 mg/kg, or inactive.

TABLE 3-2
Analytical and Biological Activity Data
C. 4′-Substituted α-Cyclohexyl-β-alanine and Precursors

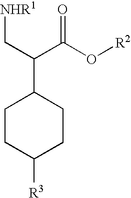

| | | | | Yield | Biological |
|---|---|---|---|---|---|
| Compound | R¹ | R² | R³ | (%)^{a} | Activity^{b} |
| B6P77 | Ac | CH₃ | H | 93.5 | NA |
| B6P81 | Ac | CH₃ | Ph | 95.8 | NA |
| B6P109 | Ac | CH₃ | C(CH₃)₃ | 98.3 | NA |
| B5P107 | H.HCl | H | Ph | 33.5 | Active - +3 |
| B5P119 | H | H | H | 51.9 | Weakly Active - |
| | | | | | +1 |
| B5P127 | H.HCl | H | C(CH₃)₃ | 62.7 | Inactive - 0 |

D. 4′-Substituted N-Acetyl-α-piperidinyl-β-alanine methyl ester

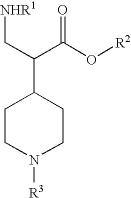

| | | | | Yield | Biological |
|---|---|---|---|---|---|
| Compound | R¹ | R² | R³ | (%) | Activity |
| B6P105 | Ac | CH₃ | CO₂Et | 96.8 | NA |

^{a}EtOH, H₂O or a mix used for recrystallizations;
^{b}Using pilocarpine, compound is active in rat at 100 mg/kg, or inactive.

TABLE 3-3
Analytical and Biological Activity Data
E. N-Acetyl-α-substituted-β-alanine methyl ester and α-Substituted-β-alanine

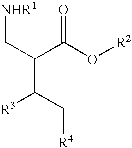

| | | | | | Yield | Biological |
|---|---|---|---|---|---|---|
| Compound | R¹ | R² | R³ | R⁴ | (%)^{a} | Activity^{b} |
| B6P85 | Ac | CH₃ | —CH₂CH₂CH₂— | | NA | NA |
| B6P93 | Ac | CH₃ | Et | CH₃ | 83.4 | NA |
| B6P97 | Ac | CH₃ | H | Bu | 99.6 | NA |

| | | | | | |
|---|---|---|---|---|---|
| B6P117 | Ac | Et | —CH₂(CH₂)₃CH₂— | 79.7 | NA |
| B6P133 | Ac | Et | —CH₂(CH₂)₈CH₂— | 98.5 | NA |
| B5P131 | H.HCl | H | —CH₂(CH₂)₈CH₂— | 36.7 | Inactive |

^{a}Yield of last synthetic step;
^{b}Using pilocarpine, compound is active in rat at 100 mg/kg, or inactive

### Example 5

The “spontaneous recurrent seizures” (SRS) model of epilepsy was used to evaluate candidate compounds in a model for Phase 1 epileptogenesis (see, e.g., Mello, E. et al., Epilepsia (1993) 34:985; Cavalheiro, J. et al., Epilepsia (1991) 32:778). In the SRS model, an adult male Sprague-Dawley rat (c. 260 g) is given pilocarpine by injection (380 mg/kg i.p.). Within 25 minutes, the animal enters status epilepticus, which typically lasts for 15-20 hours (although about 10% of animals die at this stage). The rat is allowed to spontaneously recover and is given food and water ad lib. and maintained on a 16 hour/8 hour light/dusk cycle. Rats are usually studied in groups of four. Beginning on about day 13-15, the rats develop spontaneous recurrent seizures, which occur at the rate of about 4-5 per week. The rats are videotaped 16 hours per day, and the videotapes are reviewed for behavioral seizures (including head nodding, forelimb clonus, and rearing), which are counted. The animals are watched for three months, permitting evaluation of a sufficient number of seizures. An experimental compound for evaluation can be administered at either of two times: Time 1, on Day 1, after the cessation of status epilepticus but before the onset of SRS; or Time 2, on Day 30, when the rats have been experiencing SRS for about two weeks. Administration of the candidate compound at Time 1 permits evaluation for anti-epileptogenic properties (ability to prevent the onset of seizures); administration of compounds at Time 2 permits evaluation of drugs as anti-ictogenics with the ability to suppress established seizures.

As a reference, the standard anticonvulsant phenytoin was administered (20 mg/kg/day i.v. for 10 day) at either Time 1 or Time 2. As expected, phenytoin was ineffective in preventing the onset of seizures when administered at Time 1, but was 75% effective at decreasing seizure frequency by 50% or more when administered at Time 2.

In contrast, β-alanine and an analog (α-(4-tert-butylcyclohexyl)-alanine (see Example 3) were administered at a comparable dosage (20 mg/kg/day i.v. for 10 day) at either Time 1 or Time 2 using the same protocal outlined above. At Time 1, each of these compounds was 75% effective in decreasing seizures by at least 50%; at Time 2, each compound was 50% effective in decreasing seizures by at least 50%.

The compounds of the invention listed in Tables 2 and 3, supra, were tested for biological activity per Example 7. The following compounds were found to have at least weak activity: β-p-methylphenyl-β-alanine hydrochloride, β-2-hydroxy-3-methoxyphenyl-β-alanine, β-3-methyl-4-methoxyphenyl-β-alanine (slight), β-3-(3,4-dichlorophenoxy)phenyl-β-alanine hydrochloride (moderate), β-2,5-dimethyl-4-methoxyphenyl-β-alanine, β-p-(trifluoromethoxy)phenyl-β-alanine, and β-2-fluoro-3-(trifluoromethyl)phenyl-β-alanine (moderate).

Thus, β-amino acids show activity both as anti-epileptogenic compounds and as anti-ictogenic compounds.

### Example 6

Dioxapiperazine compounds were synthesized according to standard methods and and characterized by NMR, FAB-MS, melting point, and HPLC. The crystal structures of several compounds were determined.

An exemplary procedure is as follows:

Boc-L-alanine (1.5 g, 0.008 mol) was dissolved in 60 ml ethyl acetate, to which 2.4 g 2-ethoxycarbonyl-1,2-dihydroquinoline (EEDQ) (0.010 mol, 1.2 equiv.) was added. The solution was stirred for 5 minutes, after which β-phenylglycine methyl ester HCl (1.5 g, 0.003 mol) was added. Stirring was continued for 24 hours, and then the solution was washed with 3×25 mL 10% (w/w) KHSO₄ (aq), 25 mL saturated NaCl solution, 3×25 saturated sodium bicarbonate solution, and 25 mL satuarated NaCl solution. The organic layer was dried over magnesium sulfate and evaporated to yield a clear oil. The oil was dissolved in 20 ml formic acid and stirred for two hours at room temperature. The acid was removed by evaporation and the oil was suspended in a mixture of 50 mL 2-butanol and 25 mL toluene. The mixture was refluxed for 24 hours, cooled over two hours with stirring, and the solvent reduced to above one-fourth the original volume in vacuo. The solid was allowed to crystallize. Cyclo-D-phenylglycine-L-alanine was obtained as a white solid (1.1 g, 0.005 mol, 68% yield) with a melting range of 260-265° C.

### Example 7

Selected compounds were dissolved in 0.9% NaCl or suspended in a mixture of 30% polyethylene glycol 400 and 70% water, and tested in an animal model. Briefly, the compounds were administered intraperitoneally or or orally to carsworth Farms #1 mice (in a volume of 0.01 ml/g of body weight) or Sprague-Dawley rats (in a volume of 0.004 ml/g body weight). Times on peak effect and peak neurologic deficit were determined before the anticonvulsant tests were administered.

The maximal electroshock seizure test (MES), corneal electrodes primed with a drop of electrolyte solution (0.9% NaCl) were applied to the eyes of the animal and an electrical stimulus (50 mA for mice, 150 mA for rats; 60 Hz) was delivered for 0.2 second at the time of the peak effect of the test compound. The animals were restrained by hand and released at the moment of stimulation in order to permit observation of the seizure. Abolition of hind-leg tonic-extensor component (hind-leg tonic extension does not exceed a 90° angle to the plane of the body) indicated that the compound prevented MES-induced seizure spread.

In the subcutaneous pentylenetetrazol threshold test (scMet), the convulsant dose (CD97) of pentylenetetrazol (85 mg/kg in rats) was injected at the time of peak effect of the test compound. The animals were isolated and observed for 30 minutes to see whether seizures occurred. Absence of clonic spasms persisting for at elast five seconds indicated that the compound could elevate the pentylenetetrazol induced seizure threshold.

Acute anti-convulsant drug-induced toxicity in lab animals is usually characterized by some type of neurologic abnormality. In mice, these abnormalities can be detected by the rotorod ataxia test, which is somewhat less useful in rats. In the rotorod ataxia test, neurologic deficit is indicated by the inability of the animal to maintain equilibrium for at least one minute on a knurled rod rotating at 6 rpm. Rats were examined by the positional sense test: one hind leg is gently lowered over the edge of a table, whereupon the normal animal will lift the leg back to a normal position. Inability to return the leg to normal position indicates a neurologic deficit.

### Example 8

Testing of the dioxapiperazine compounds was performed in 12 mice at doses of 30, 100, 300 mg/kg (4 mice each) 30 minutes and four hours after the test compounds was administered. The results are shown in Table 4.

TABLE 4
Selected Dioxapiperazine Compounds and Testing data.

| | Activity: | Activity: | Activity: |
|---|---|---|---|
| Compound | 300 mg/kg | 100 mg/kg | 30 mg/kg |
| c/D-Peg-L-Ala | 4 | 3 | 2 |
| c/L-Peg-L-Ala | 0 | 0 | NA |
| c/D-Peg-Gly | 2 | 1 | 0 |
| c/D-Peg-L-Lys | 1 | 0 | NA |
| c/D-Peg-D-Lys | 0 | 0 | NA |
| c/D-Peg-L- | 0 | 0 | NA |
| Ornithine (Orn) | | | |
| c/D-Peg-D-Orn | 0 | 0 | NA |
| c/D-Peg-L- | 0 | 0 | NA |
| diaminobutyric acid | | | |
| c/D-Peg-L- | 0 | 0 | NA |
| diaminopropionic | | | |
| acid | | | |
| c/D-Peg-L-Met | 1 | 0 | NA |
| c/D-Peg-D-Met | 0 | 0 | NA |
| c/D-Peg-L-(S- | 4 | 3 | 2 |
| methyl)-L-cysteine | | | |
| c/D-Peg-L-(S- | 0 | 0 | NA |
| benzyl)-L-cysteine | | | |
| c/D-Peg-L-Arg | 0 | 0 | NA |
| c/D-Peg-L- | 0 | 0 | NA |
| HomoArg | | | |
| c/D-Peg-N- | 0 | 0 | NA |
| guanidine-L- | | | |
| homoArg | | | |
| c/D-(p-OH)-Peg-L- | 0 | 0 | NA |
| Ala | | | |
| c/D-(p-OH)-Peg-L- | 0 | 0 | NA |
| Lys | | | |

c = cyclo
Peg = phenylglycine
Activity on scale of 0 (inactive) to 4.

As seen in Table 4, c/D-phenylglycine-L-alanine and c/D-phenylglycine-(S-Me)-L-cysteine exhibited strong anti-convulsive activity in this animal model system, while several other dioxapiperazines showed weaker anti-convulsive activity.

Certain other diozapiperazines were also synthesized and tested. Of these compounds, c/L-alanine-D-leucine was found to be active.

### Example 9

### Biaryl Ether Anti-Epileptogenic Agents

In still another embodiment, a method for inhibiting epileptogenesis and/or ictogenesis in a subject involves administering to a subject an effective amount of a compound such that epileptogenesis is inhibited, where the compound is

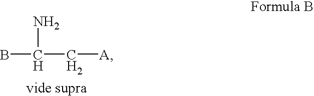

More particularly, preferred compounds are of the formula:

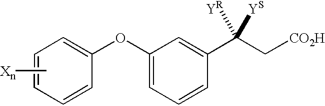

wherein each X is independently selected from the group consisting of halogen (chloro preferred), nitro, cyano, and substituted or unsubstituted alkyl and alkoxy groups (trifluoromethyl and methyl preferred); n is an integer from 0 to 5 (n=1 preferred); and one of Y^{R} and Y^{S} is a hydrogen, and the other is a substituted or unsubstituted amine, including pharmaceutically acceptable salts thereof.

TABLE 5
Example Biaryl Ether Compounds

| | | | | | Biological |
|---|---|---|---|---|---|
| Compound | X | n | Y^{R} | Y^{S} | Activity^{a} |
| C1 | m-CF₃ | 1 | NH₂. HCl | H | Inactive - 0 |
| C2 | m-CF₃ | 1 | H | NH₂.HCl | Active - +1 |
| | m-CF₃ | 1 | NH₂.HCl, H (racemate) | | Active - +3 |
| C3 | p-CH₃ | 1 | NH₂.HCl | H | Active - +1 |
| C4 | p-CH₃ | 1 | H | NH₂.HCl | Active - +2 |
| | p-CH₃ | 1 | NH₂.HCl, H (racemate) | | Inactive - 0 |
| C5 | — | 0 | NH₂.HCl | H | Active - +1 |
| C6 | — | 0 | NH₂.HCl, H (racemate) | | Active - +1 |
| C7 | — | 0 | H | NH₂.HCl | Active - +1 |
| C8 | p-Cl | 1 | H | NH₂.HCl | Active - +1 |
| C9 | p-Cl | 1 | NH₂.HCl | H | Active - +2 |
| C10 | m-Cl, p-Cl | 2 | NH₂.HCl | H | NA |
| C11 | m-Cl, p-Cl | 2 | H | NH₂.HCl | NA |

^{a}Using pilocarpine, compound is active in rat at 100 mg/kg, or inactive.

Alternatively, the biarylether may be para-substituted:

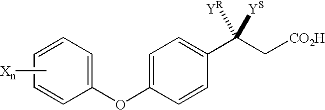

For example, see compound B8P79 in Table 2, supra.

As the biological data indicate, the enantiomer of either R or S absolute stereochemistry may be more biologically active than the racemate or the other stereoisomer. When this is the case, that single stereoisomer is preferred, and pharmaceutical compositions according to the invention preferrably comprise substantially only that stereoisomer. Such stereochemical isomer may be prepared either by asymmetric synthesis from chiral starting materials (e.g., by Michael addition of a chiral amine to a cinnamate ester followed by hydrolysis), or by resolution of a racemic synthesis, as exemplified below.

Methyl 3-(3-trifluoromethylphenoxy)-trans-cinnamate. A solution of 3-[3-(trifluoromethyl)phenoxy]benzaldehyde (8.05 g, 30 mmol) and methyltriphenylphosphoranylidene acetate (15.13 g, 45 mmol) in THF (200 mL) was stirred at reflux for 24 h, then cooled to room temperature, concentrated. Purification of the residue by chromatography on silica gel with an eluant of 0-10% EtOAc in hexane provided 9.3 g (96%).

Methyl 3-(4-methylphenoxy)-trans-cinnamate. A solution of 3-(4-methylphenoxy)benzaldehyde (8.04 g, 37.9 mmol) and methyltriphenylphosphoranylidene acetate (19 g, 57 mmol) in THF (200 mL) was stirred at reflux for 24 h, then cooled to room temperature, concentrated. Purification of the residue by chromatography on silica gel with an eluant of 0-10% EtOAc in hexane provided 9.6 g (94.5%).

Methyl 3-phenoxy-trans-cinnamate. A solution of 3-phenoxybenzaldehyde (8.03 g, 40.5 mmol) and methyltriphenylphosphoranylidene acetate (20 g, 60 mmol) in THF (200 mL) was stirred at reflux for 24 h, then cooled to room temperature, concentrated. Purification of the residue by chromatography on silica gel with an eluant of 0-10% EtOAc in hexane provided 10.2 g (99%).

Methyl (3R)-[(S)-(−)-N-benzyl-α-methylbenzyl]amino-3-[3-(3-trifluoromethylphenoxy)phenyl]propanoate. Butyl lithium (2.5 M in hexane, 9.9 mL, 24.75 mmol) was added to (S)-(−)-N-benzyl-α-methylbenzylamine (5.3 mL, 25 mmol) in THF (200 mL) at 0° C. The red solution was stirred at 0° C. for 20 min and cooled to −78° C. Methyl 3-(3-trifluoromethylphenoxy)-trans-cinnamate (4 g, 12.4 mmol) in THF (20 mL) was added dropwise. The mixture was stirred for 2 h at −78° C. before quenching with saturated ammonium chloride (100 mL), then allowed to warm and poured into saturated aqueous sodium chloride solution (100 mL). Extraction of the aqueous layer with EtOAc (2×100 mL), drying (Na₂SO₄), filtration and evaporation gave a residue that was purified by chromatography on silica gel with an eluant of 0-8% EtOAc in hexane. Evaporation of the collected fractions provided 3.2 g (47%).

Methyl (3S)-[(R)-(+)-N-benzyl-α-methylbenzyl]amino-3-[3-(3-trifluoromethylphenoxy)phenyl]propanoate (4.1 g) was prepared by the same procedure from (R)-(+)-N-benzyl-α-methylbenzylamine in 62% yield.

Methyl (3R)-[(S)-(−)-N-benzyl-α-methylbenzyl]amino-3-[3-(3-trifluoromethylphenoxy)phenyl]propanoate. Butyl lithium (2.5 M in hexane, 12 mL, 30 mmol) was added to (S)-(−)-N-benzyl-α-methylbenzylamine (6.3 mL, 30 mmol) in THF (200 mL) at 0° C. The red solution was stirred at 0° C. for 20 min and cooled to −78° C. Methyl 3-(3-trifluoromethylphenoxy)-trans-cinnamate (4 g, 14.9 mmol) in THF (20 mL) was added dropwise. The mixture was stirred for 2 h at −78° C. before quenching with saturated ammonium chloride (100 mL), then allowed to warm and poured into saturated aqueous sodium chloride solution (100 mL). Extraction of the aqueous layer with EtOAc (2×100 mL), drying (Na₂SO₄), filtration and evaporation gave a residue that was purified by chromatography on silica gel with an eluant of 0-8% EtOAc in hexane. Evaporation of the collected fractions provided 3.3 g (46%).

Methyl (3S)-[(R)-(+)-N-benzyl-α-methylbenzyl]amino-3-[3-(3-trifluoromethylphenoxy)phenyl]propanoate (4.4 g) was prepared by the same procedure from (R)-(+)-N-benzyl-α-methylbenzylamine in 62% yield.

Methyl (3R)-[(S)-(−)-N-benzyl-α-methylbenzyl]amino-(3-phenoxyphenyl)propanoate. Butyl lithium (2.5 M in hexane, 13 mL, 32.5 mmol) was added to (S)-(−)-N-benzyl-α-methylbenzylamine (6.6 mL, 31.6 mmol) in THF (200 mL) at 0° C. The red solution was stirred at 0° C. for 20 min and cooled to −78° C. Methyl 3-(4-methylphenoxy)-trans-cinnamate (4 g, 15.7 mmol) in THF (20 mL) was added dropwise. The mixture was stirred for 2 h at −78° C. before quenching with saturated ammonium chloride (100 mL), then allowed to warm and poured into saturated aqueous sodium chloride solution (100 mL). Extraction of the aqueous layer with EtOAc (2×100 mL), drying (Na₂SO₄), filtration and evaporation gave a residue that was purified by chromatography on silica gel with an eluant of 0-8% EtOAc in hexane. Evaporation of the collected fractions provided 4.8 g (66%).

Methyl (3S)-[(R)-(+)-N-benzyl-α-methylbenzyl]amino-3-[3-(3-trifluoromethylphenoxy)phenyl]propanoate was prepared by the same procedure from (R)-(+)-N-benzyl-α-methylbenzylamine in 51% yield.

Methyl (3R)-Amino-3-[3-(3-trifluoromethylphenoxy)phenyl]propanoate. The solution of Methyl (3R)-[(S)-(−)-N-benzyl-α-methylbenzyl]amino-3-[3-(3-trifluoromethylphenoxy)phenyl]propanoate (3.2 g, 5.8 mmol) in MeOH (60 mL), H₂O (6 mL) and acetic acid (1.5 mL) in the presence of palladium hydroxide on charcoal (700 mg) under hydrogen (1 atm) was stirred at room temperature for 36 h. Filtration and evaporation to give product. The product was used without purification in the next reaction.

Methyl (3S)-Amino-3-[3-(3-trifluoromethylphenoxy)phenyl]propanoate was prepared by the same procedure from (3R)-[(R)-(+)-N-benzyl-α-methylbenzyl]amino-3-[3-(3-trifluoromethylphenoxy)phenyl]propanoate (3.9 g, 7.1 mmol).

Methyl (3R)-Amino-3-[3-(4-methylphenoxy)phenyl]propanoate. The solution of Methyl (3R)-[(S)-(−)-N-benzyl-α-methylbenzyl]amino-3-[3-(4-methylphenoxy)phenyl]propanoate (3.3 g, 6.7 mmol) in MeOH (60 mL), H₂O (6 mL) and acetic acid (1.5 mL) in the presence of palladium hydroxide on charcoal (530 mg) under hydrogen (1 atm) was stirred at room temperature for 36 h. Filtration and evaporation to give product. The product was used without purification in the next reaction.

Methyl (3S)-Amino-3-[3-(3-trifluoromethylphenoxy)phenyl]propanoate was prepared by the same procedure from (3R)-[(R)-(+)-N-benzyl-α-methylbenzyl]amino-3-[3-(4-methylphenoxy)phenyl]propanoate (4.2 g, 8.5 mmol).

Methyl (3R)-Amino-3-(3-phenoxyphenyl)propanoate. The solution of Methyl (3R)-[(S)-(−)-N-benzyl-α-methylbenzyl]amino-3-(3-phenoxyphenyl)propanoate (4.4 g, 9.1 mmol) in MeOH (60 mL), H₂O (6 mL) and acetic acid (1.5 mL) in the presence of palladium hydroxide on charcoal (700 mg) under hydrogen (1 atm) was stirred at room temperature for 36 h. Filtration and evaporation to give product. The product was used without purification in the next reaction.

Methyl (3S)-Amino-3-(3-phenoxyphenyl)propanoate was prepared by the same procedure from (3S)-[(R)-(+)-N-benzyl-α-methylbenzyl]amino-3-(3-phenoxyphenyl)propanoate (3.7 g, 7.7 mmol).

(3R)-Amino-3-[3-(3-trifluoromethylphenoxy)phenyl]propionic acid hydrochloride (C1). Methyl (3R)-Amino-3-[3-(3-trifluoromethylphenoxy)phenyl]propanoate was dissolved in 2N HCl (40 mL), reflux for overnight, cooled to room temperature and concentrated. The residue was dissolved in 2N HCl (100 mL) and diethyl ether (30 mL). The oil layer formed between aqueous and organic layer and was separated, evaporated and dried on pump overnight to give white powder 1.8 g: [α]²⁰_{D}−0.49° (c 2.26, CH₃OH), ¹H NMR (CD₃OD) δ2.99 (dd, 1H, J=6.6, 17.4), 3.09 (dd, 1H, J=7.5, 17.4), 4.72 (dd, 1H, J=6.6, 7.5), 7.08-7.60 (m, 8H). ¹³C NMR (CD₃OD) δ39.1, 52.8, 116.3, 119.7, 121.3, 123.3, 123.4, 124.2, 127.0, 132.2, 132.3, 133.4, 140.0, 158.2, 159.0, 172.8. MS: m/e 326.0 (m-HCl).

(3S)-Amino-3-[3-(3-trifluoromethylphenoxy)phenyl]propionic acid hydrochloride (C2) was prepared by the same procedure from Methyl (3S)-Amino-3-[3-(3-trifluoromethylphenoxy)phenyl]propanoate in 74% yield. [α]²⁰_{D}+0.63° (c 2.38, CH₃OH), ¹H NMR (CD₃OD) δ2.99 (dd, 1H, J=6.6, 17.4), 3.09 (dd, 1H, J=7.5, 17.4), 4.72 (dd, 1H, J=6.6, 7.5), 7.08-7.60 (m, 8H). ¹³C NMR (CD₃OD) δ39.1, 52.8, 116.3, 119.7, 121.3, 123.3, 123.4, 124.2, 127.0, 132.2, 132.3, 133.4, 140.0, 158.2, 159.0, 172.8. MS: m/e 326.3 (m-HCl).

(3R)-Amino-3-[3-(4-methylphenoxy)phenyl]propionic acid hydrochloride (C3). Methyl (3R)-Amino-3-[3-(4-methylphenoxy)phenyl]propanoate was dissolved in 2N HCl (40 mL), reflux for overnight, cooled to room temperature and concentrated. The residue was dissolved in 2N HCl (100 mL), concentrated. The white precipitate was filtrated and washed with diethyl ether (10 mL) and dried on pump overnight to give 1.6 g: [α]²⁰_{D}−1.36° (c 2.06, CH₃OH), ¹H NMR (CD₃OD) δ2.88 (s, 1H), 2.96 (dd, 1H, J=6.6, 17.1), 3.09 (dd, 1H, J=7.8, 17.1), 4.67 (dd, 1H, J=6.6, 7.8), 6.89-7.43 (m, 8H). ¹³C NMR (CD₃OD) δ20.8, 39.1, 52.9, 118.1, 119.8, 120.4, 122.6, 131.5, 131.8, 134.8, 139.4, 155.5, 160.0, 172.8. MS: m/e 272.0 (m-HCl).

(3S)-Amino-3-[3-(4-methylphenoxy)phenyl]propionic acid hydrochloride (C4) was prepared by the same procedure from Methyl (3S)-Amino-3-[3-(4-methylphenoxy)phenyl]propanoate in 65% yield: [α]²⁰_{D}+1.46° (c 2.26, CH₃OH), ¹H NMR (CD₃OD) δ2.88 (s, 1H), 2.96 (dd, 1H, J=6.6, 17.1), 3.09 (dd, 1H, J=7.8, 17.1), 4.67 (dd, 1H, J=6.6, 7.8), 6.89-7.43 (m, 8H). ¹³C NMR (CD₃OD) δ20.8, 39.1, 52.9, 118.1, 119.8, 120.4, 122.6, 131.5, 131.8, 134.8, 139.4, 155.5, 160.0, 172.8. MS: m/e 272.1 (m-HCl).

(3R)-Amino-3-(3-phenoxyphenyl)propionic acid hydrochloride (C5). Methyl (3R)-Amino-3-(3-phenoxy)phenylpropanoate was dissolved in 2N HCl (40 mL), reflux for overnight, cooled to room temperature and concentrated. The residue was dissolved in 2N HCl (100 mL), washed with diethyl ether (2×30 mL). The aqueous was evaporated and dried on pump overnight to give white powder 2.4 g: [α]²⁰_{D}−1.40° (c 2.79, CH₃OH), ¹H NMR (CD₃OD) δ2.98(dd, 1H, J=6.6, 17.1),3.11 (dd, 1H, J=7.5, 17.1), 4.69(dd, 1H, J=6.6, 7.5), 6.98-7.46 (m, 9H). ¹³C NMR (CD₃OD) δ39.1, 52.8, 118.7, 120.2, 120.3, 123.0, 125.0, 131.1, 131.9, 139.5, 158.0, 159.4, 172.8. MS: m/e 258.1 (m-HCl).

(3S)-Amino-3-(3-phenoxyphenyl)propionic acid hydrochloride (C7) (1.96 g) was prepared by the same procedure from Methyl (3S)-Amino-3-(3-phenoxyphenyl)propanoate in 87% yield: [α]²⁰_{D}+1.43° (c 2.25, CH₃OH), ¹H NMR (CD₃OD) δ2.98 (dd, 1H, J=6.6, 17.1), 3.11 (dd, 1H, J=7.5, 17.1), 4.69 (dd, 1H, J=6.6, 7.5), 6.98-7.46 (m, 9H). ¹³C NMR (CD₃OD) δ39.1, 52.8, 118.7, 120.2, 120.3, 123.0, 125.0, 131.1, 131.9, 139.5, 158.0, 159.4, 172.8. MS: m/e 257.9 (m-HCl).

(D)-(+)-3-amino-3-[3-(4-chlorophenoxy)phenyl]propionic acid, hydrochloride (C8) and (L)-(−)-3-amino-3-[3-(4-chlorophenoxy)phenyl]propionic acid, hydrochloride (C9) were produced from diastereomeric selective recrystalization of BOC-protected racemic 3-amino-3-[3-(4-chlorophenoxy)phenyl]propionic acid with (1R,2S)-(−)-ephedrine in EtOAc, followed by acidic removal of the BOC group using art-recognized techniques. The specific rotations of these compounds were +1.07° and −1.04° (c=0.0118 in MeOH). The ¹H and ¹³C NMR were consistent with the structures.

(L)-(−)-3-amino-3-[3-(3,4-dichlorophenoxy)phenyl]propionic acid, hydrochloride (C10) and (D)-(+)-3-amino-3-[3-(3,4-dichlorophenoxy)phenyl]propionic acid, hydrochloride (C1) were prepared by enzymatic resolution. Racemic 3-[3-(3,4-dichloro-phenoxy)-phenyl]-3-phenylacetylamino-propionic acid (2.01 g, 4.5 mmol), prepared from the reaction of phenylacetyl chloride and 3-amino-3-[3-(3,4-dichloro-phenoxy)-phenyl]-propionic acid, was dissolved in 30 mL of EtOAc. To this solution was added 30 mL of a 1M phosphate buffer (pH=7.6) and 200 mg (10% w/w) of penicillin G amidase (PGA) immobilized on Eupergit. The reaction was stopped after 24 h, and the enzyme was removed by filtration. Amine and acetimamide products were separated by partitioning between EtOAc and aqueous acid, and solvent was removed under reduced pressure and with the freeze drierproducing 198 mg (24%) of enriched (L)-(−)-compound ([α]_{D}=−0.39°, c=0.0058 in MeOH). The ¹H and ¹³C NMR were consistent with the structure. ion was stopped after 24 h. Further hydrolysis of the acetamide with 6M HCl produced 970 mg (79%) of enriched (D)-(+)-compound ([α]_{D}=+0.13° (c=0.0173 in MeOH). The ¹H and ¹³C NMR were consistent with the structure.

### EQUIVALENTS

Those skilled in the art will recognize, or be able to ascertain using no more than routine experimentation, numerous equivalents to the specific procedures described herein. Such equivalents are considered to be within the scope of the present invention and are covered by the following claims. The contents of all references, issued patents, and published patent applications cited throughout this application are hereby incorporated by reference. The appropriate components, processes, and methods of those patents, applications and other documents may be selected for the present invention and embodiments thereof.

What is claimed is:

1. A method for identifying a compound which inhibits epileptogenesis in a subject, comprising the steps of:
i) obtaining the structures of two or more compounds each having
a) the ability to cause a direct or an indirect pharmacological effect on a polypeptide which is involved in epileptogenesis, and
b) a pharmacophore which has been determined to exert at least some of said pharmacological effect,
ii) determining an average pharmacophore structure based on the structures of the pharmacophores of said two or more compounds, and
iii) choosing a new compound which comprises the average pharmacophore.

2. A method for identifying a compound which inhibits epileptogenesis in a subject, comprising:
i) obtaining the structures of two or more compounds each having
a) the ability to cause a direct or an indirect pharmacological effect on a polypeptide which is involved in epileptogenesis, and
b) a pharmacophore which has been determined to exert at least some of said pharmacological effect,
ii) determining an average pharmacophore structure based on the structures of the pharmacophores of said two or more compounds,
iii) repeating at least once steps (i) and (ii) for a different polypeptide which is involved in epileptogenesis, and
iv) choosing a new compound which comprises one or more average pharmacophore determined in the previous steps.

3. The method of claim 1, wherein said pharmacological activity on a polypeptide which is involved in epileptogenesis is chosen from the group consisting of inhibition, agonism, antagonism, chelation, and binding.

4. The method of claim 1, wherein said structure is a carbon backbone structure.

5. The method of claim 1, wherein said structure is a three dimensional space-filling structure.

6. The method of claim 1, wherein said polypeptide which is involved in epileptogenesis is a cell-surface receptor.

7. The method of claim 6, wherein said polypeptide which is involved in epileptogenesis is an NMDA receptor.

8. The method of claim 1, wherein said polypeptide which is involved in epileptogenesis is involved in transport of a neurotransmitter.

9. The method of claim 8, wherein said polypeptide which is involved in epileptogenesis is a GABA transporter.

10. A method for inhibiting epileptogenesis in a subject, comprising administering to a subject an effective amount of a compound which inhibits epileptogenesis and which has been identified with the method of claim 1.

11. A method for inhibiting epileptogenesis in a subject, comprising administering to a subject an effective amount of a compound such that epileptogenesis is inhibited, wherein the compound is of Formula A 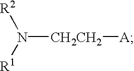
i) where R¹ is hydrogen, alkyl, alkenyl, alkynyl, aryl, alkylcarbonyl, arylcarbonyl, alkoxycarbonyl, or aryloxycarbonyl;
ii) R² is alkyl, alkenyl, alkynyl, aryl, alkylcarbonyl, arylcarbonyl, alkoxycarbonyl, or aryloxycarbonyl;
iii) A is an anionic group at physiological pH;
and pharmaceutically acceptable salts or esters thereof.

12. The method of claim 11, where A is carboxyl.

13. The method of claim 12, where R¹ is hydrogen.

14. The method of claim 13, where R² is alkyl.

15. The method of claim 14, where R² is arylalkyl.

16. The method of claim 15, where R² is phenylalkyl.

17. The method of claim 11, where said compound is selected from the group consisting of 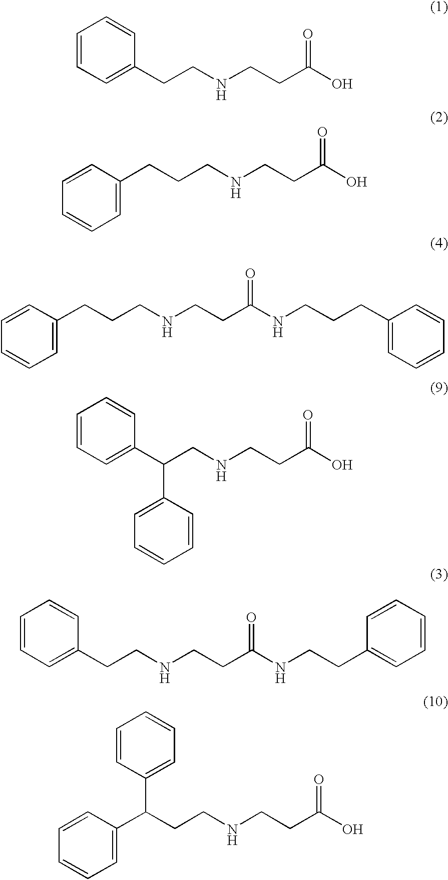
and pharmaceutically acceptable salts or esters thereof.

18. A method for inhibiting epileptogenesis in a subject, comprising administering to a subject an effective amount of a compound such that epileptogenesis is inhibited, where said compound is of Formula B 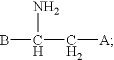
i) wherein A is an anionic group at physiological pH;
ii) wherein B is a phenoxy substituted phenyl group;
and pharmaceutically acceptable salts or esters thereof.

19. The method of claim 18, where A is a carboxyl group.

20. The method of claim 19, where B is an alkylphenoxy substituted phenyl group.

21. The method of claim 20, where B is a methylphenoxy substituted phenyl group.

22. The method of claim 19, where B is a halophenoxy substituted phenyl group.

23. The method of claim 22, where B is a chlorophenoxy substituted phenyl group.

24. The method of claim 18, where said compound is selected from the group consisting of 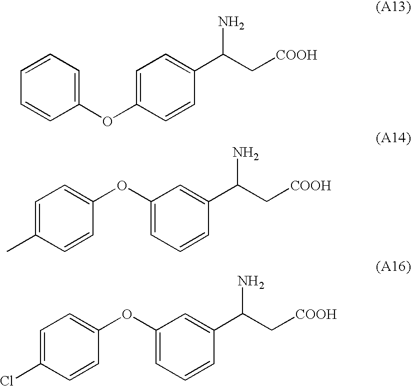
and pharmaceutically acceptable salts or esters thereof.

25. A method for inhibiting epileptogenesis in a subject, comprising administering to a subject an effective amount of a compound such that epileptogenesis is inhibited, wherein the compound is of Formula C 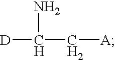
i) wherein A is an anionic group at physiological pH;
ii) wherein D is an aryl group substituted with 2 or more moieties selected from the group consisting of alkoxy and aryloxy;
and pharmaceutically acceptable salts thereof.

26. The method of claim 25, where A is a carboxyl group.

27. The method of claim 26, where D is a phenyl group substituted with 2 or more moieties selected from the group consisting of alkoxy and aryloxy.

28. The method of claim 27, where D is a phenyl group substituted with 2 or more alkoxy groups.

29. The method of claim 28, where the alkoxy groups are methoxy groups.

30. The method of claim 25, where said compound is selected from the group consisting of 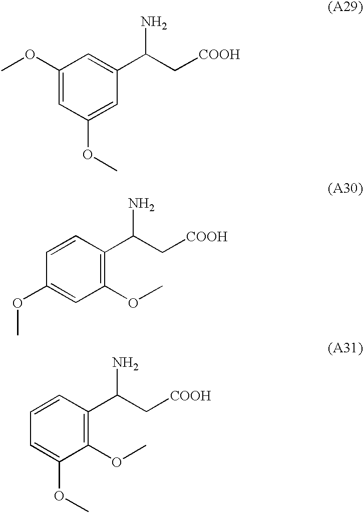
and pharmaceutically acceptable salts thereof.

31. A method for inhibiting epileptogenesis in a subject, comprising administering to a subject an effective amount of a compound such that epileptogenesis is inhibited, wherein the compound is of Formula D 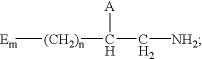
i) wherein A is an anionic group at physiological pH;
ii) m and n are independently 1, 2 or 3;
iii) E is a substituted or unsubstituted phenyl;
and pharmaceutically acceptable salts thereof.

32. The method of claim 31, where A is a carboxyl group.

33. The method of claim 32, where n is 2.

34. The method of claim 32, where n is 1.

35. The method of claim 34, where E is a diphenyl substituted methyl.

36. The method of claim 31, where said compound is selected from the group consisting of 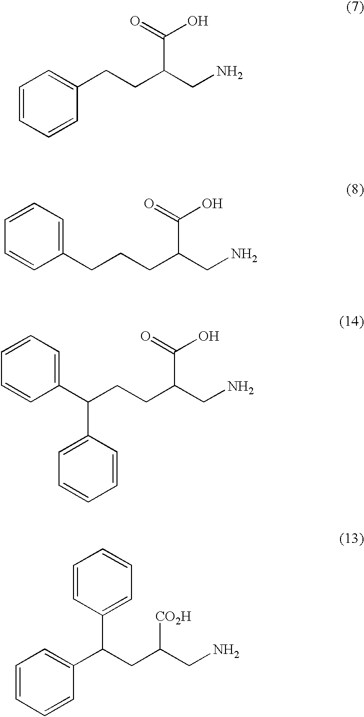
and pharmaceutically acceptable salts or esters thereof.

37. A method for inhibiting epileptogenesis in a subject, comprising administering to a subject an effective amount of a compound such that epileptogenesis is inhibited in the subject, where said compound is selected from the group consisting of 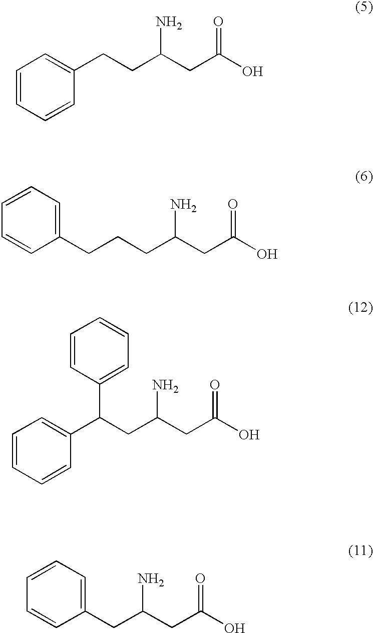
and pharmaceutically acceptable salts thereof, such that epileptogenesis is inhibited in the subject.

38. A method for inhibiting epileptogenesis in a subject, comprising administering to a subject an effective amount of a compound such that epileptogenesis is inhibited in the subject, wherein said compound is selected from the group consisting of 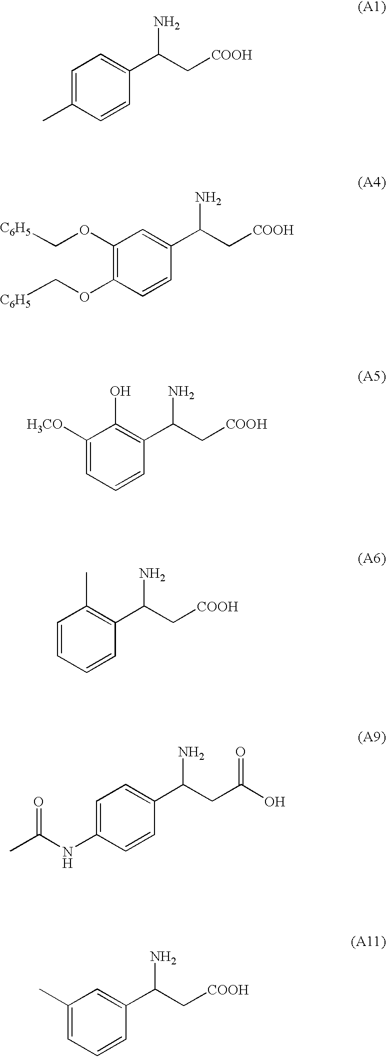 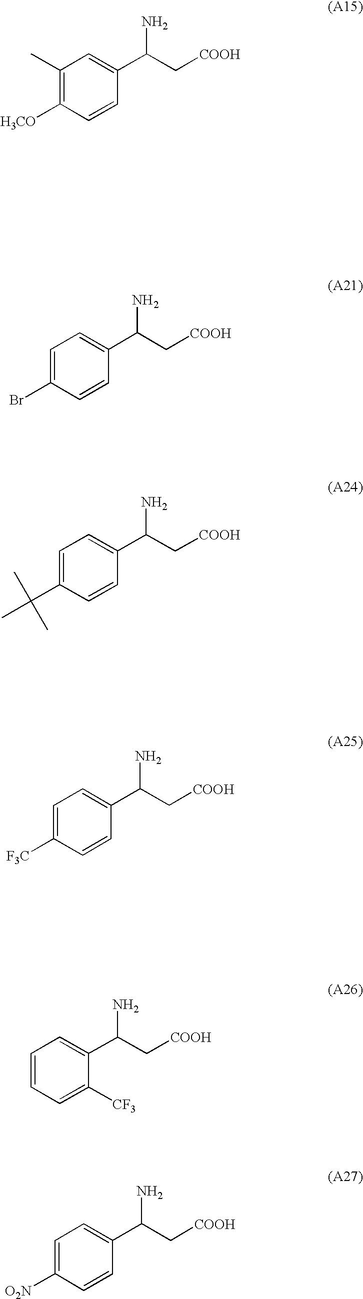 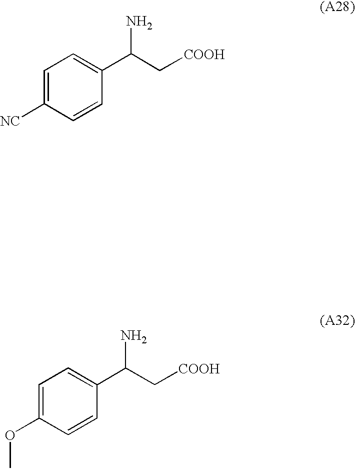
and pharmaceutically acceptable salts or esters thereof.

39. A method for inhibiting epileptogenesis in a subject, comprising administering to a subject an effective amount of a compound selected from the group consisting of α-α-disubstituted β-alanines, α,β-disubstituted β-alanines, β,β-disubstituted β-alanines, α,β,α-trisubstituted β-alanines, α,β,β-trisubstituted β-alanines, α,α,N-trisubstituted β-alanines, α,β,N-trisubstituted β-alanines, β,β,N-trisubstituted β-alanines, α,α,N,N-tetrasubstituted β-alanines, α,β,N,N-tetrasubstituted β-alanines, β,β,N,N-tetrasubstituted β-alanines, α,α,β,β-tetrasubstituted β-alanines, α,α,β,N-tetrasubstituted β-alanines, α,β,β,N-tetrasubstituted β-alanines, α,α,β,N,N-pentasubstituted β-alanines, α,β,β,N,N-pentasubstituted β-alanines, α,α,β,β,N-pentasubstituted β-alanines, α,α,β,β,N,N-hexasubstituted β-alanines, and pharmaceutically acceptable salts or esters thereof, such that epileptogenesis is inhibited in the subject.

40. A method of diagnosing an epileptogenic condition in a subject comprising: administering a compound selected from the group consisting of 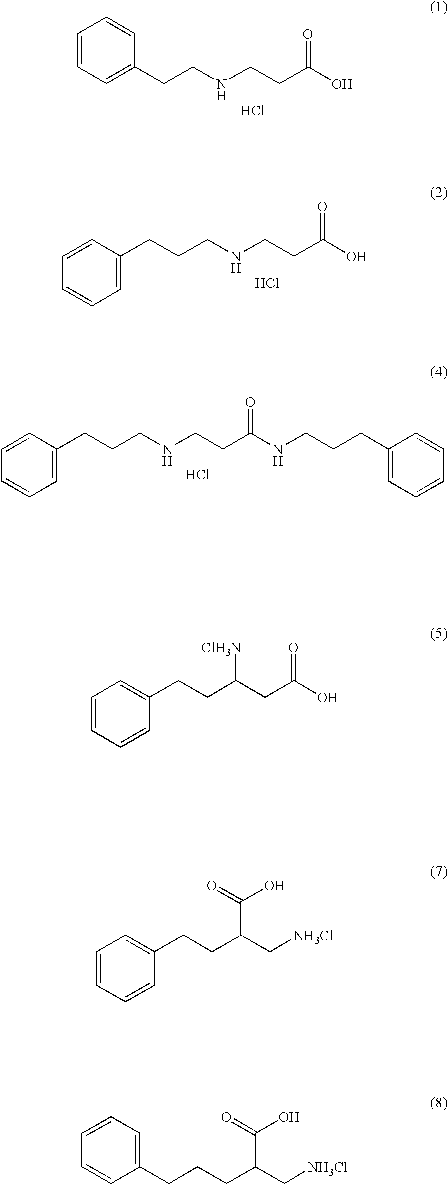 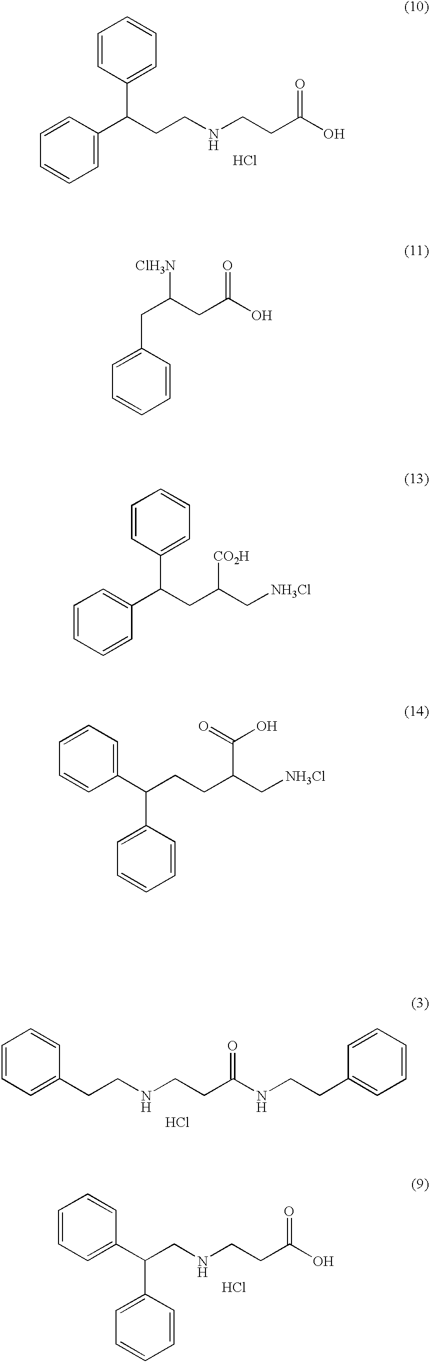 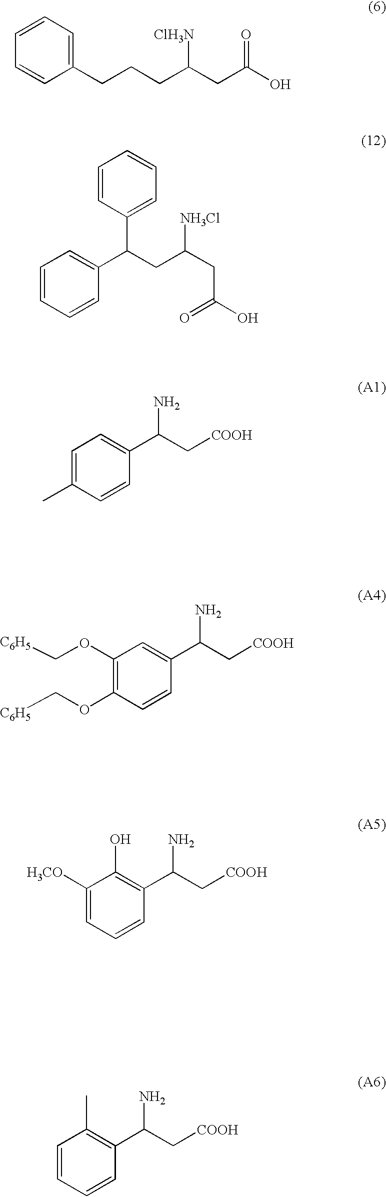 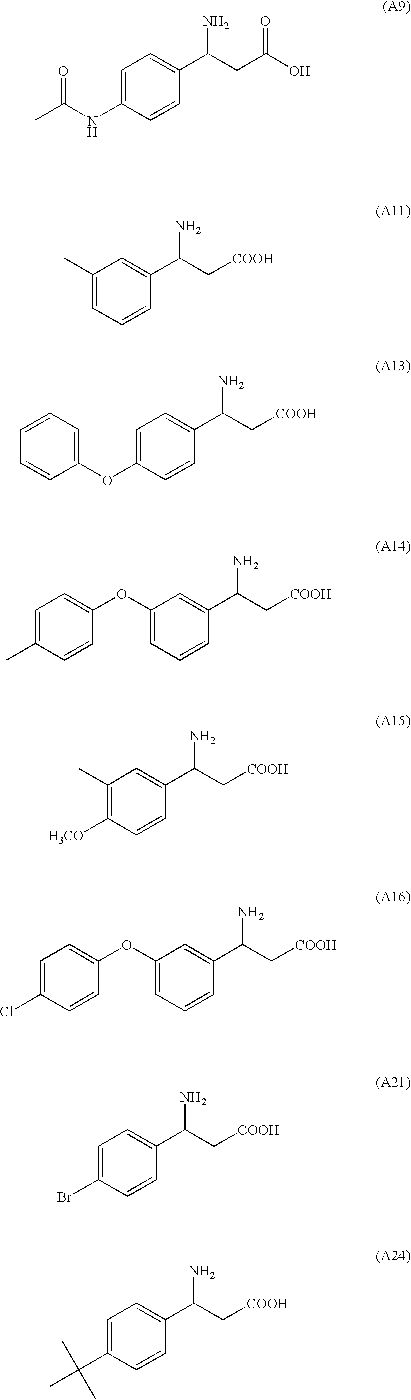 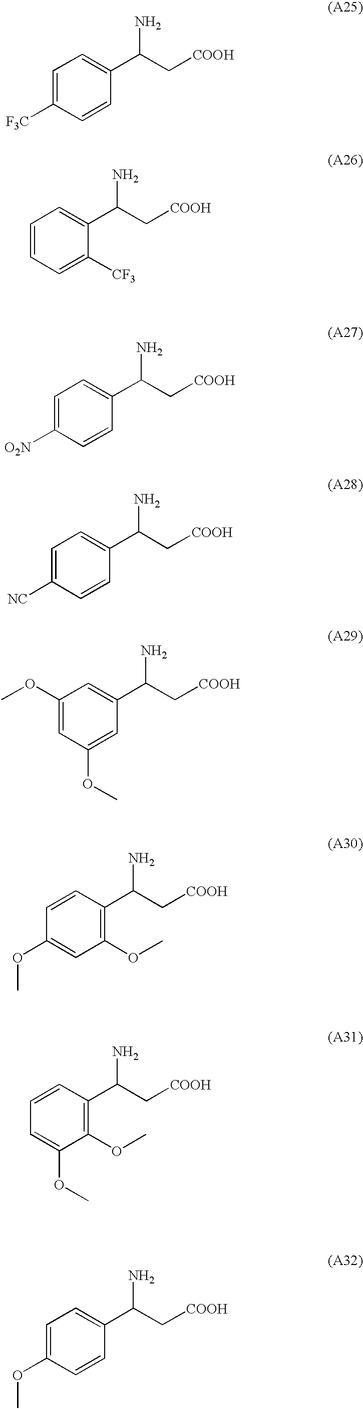
labeled with a detectable marker to said subject;
measuring increased binding of the compound to the NMDA receptors of the neurons of said subject's brain, thereby diagnosing an epileptogenic condition in said subject.

41. A method of diagnosing an epileptogenic condition in a subject comprising: administering a compound selected from the group consisting of 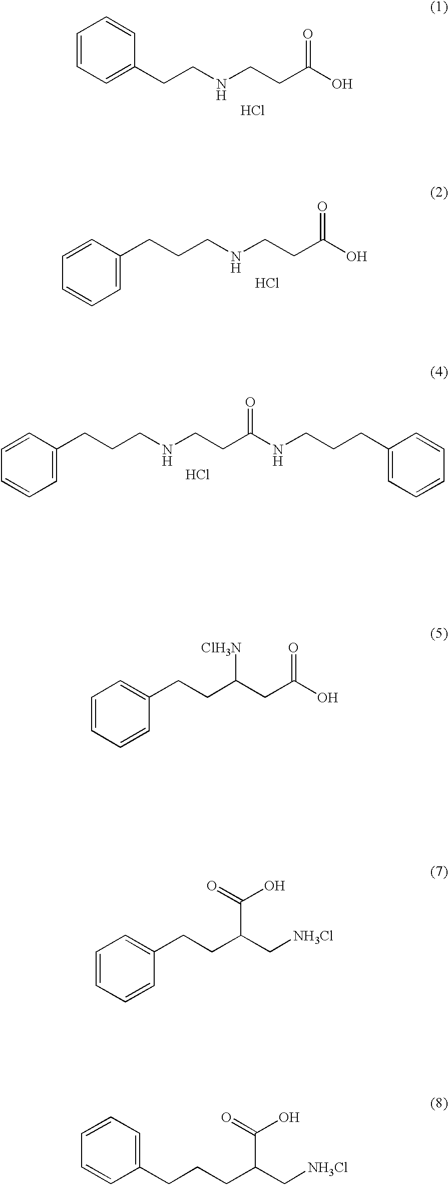 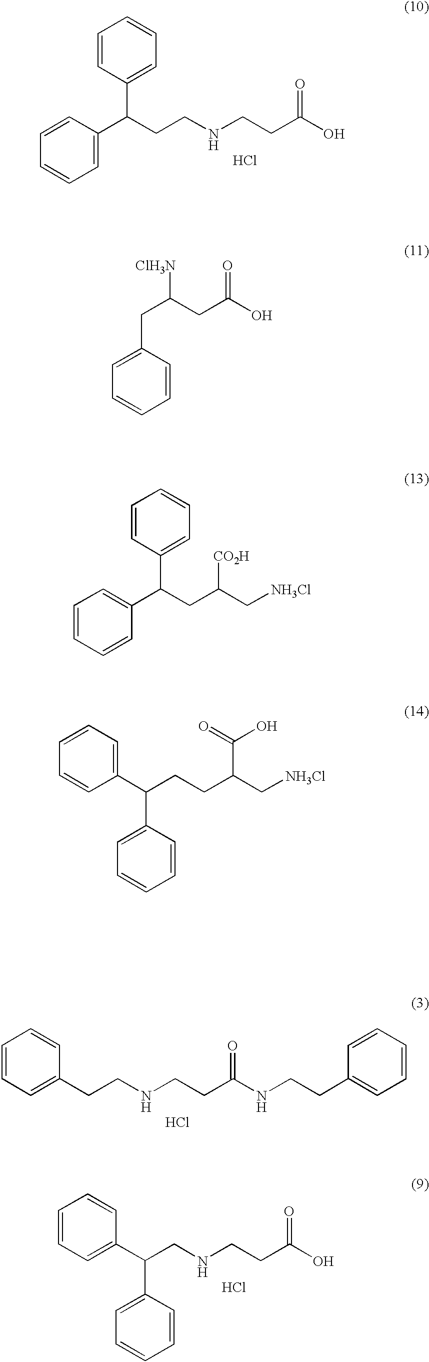 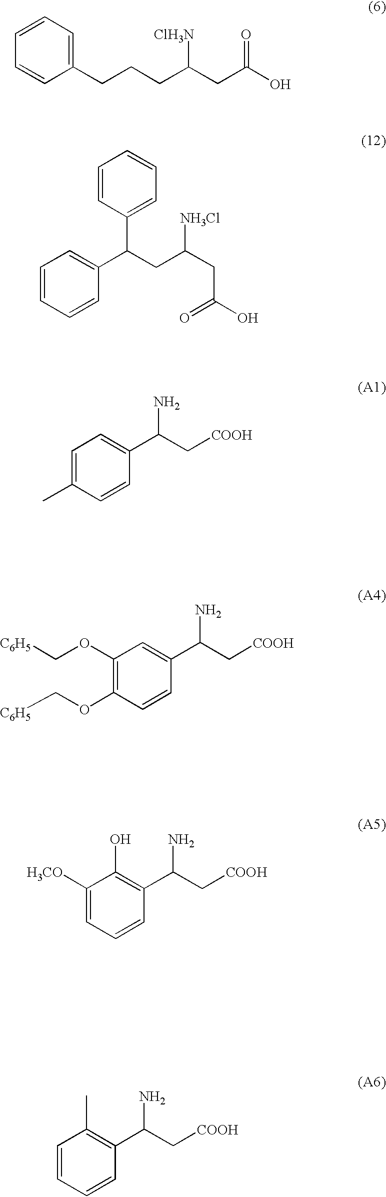 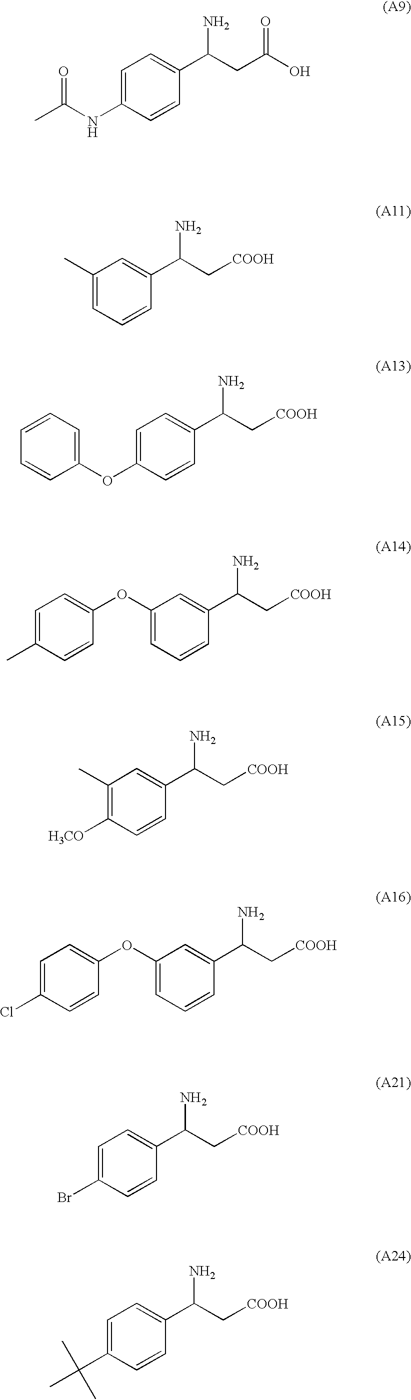 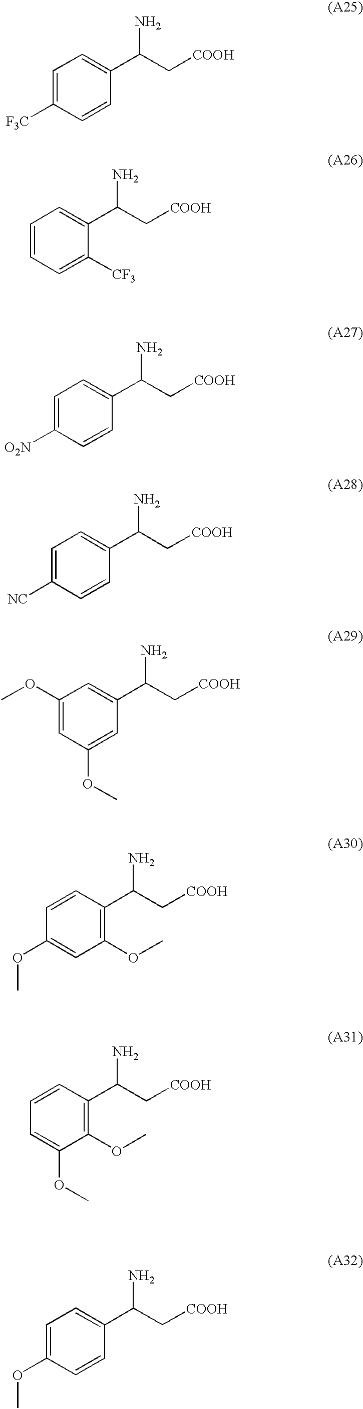
labeled with a detectable marker to said subject;
measuring decreased binding of the compound to the GABA receptors of the neurons of said subject's brain, thereby
diagnosing an epileptogenic condition in said subject.

42. A method of diagnosing an epileptogenic condition in a subject comprising administering a compound selected from the group consisting of 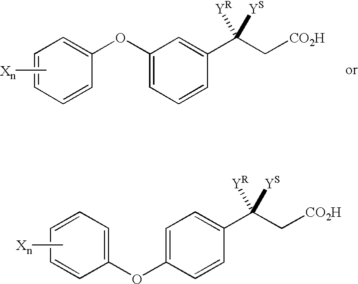
wherein each X is independently selected from the group consisting of halogen, nitro, cyano, and substituted or unsubstituted alkyl and alkoxy groups; n is an integer from 0 to 5; and one of Y^{R} and Y^{S} is a hydrogen, and the other is a substituted or unsubstituted amine; and pharmaceutically acceptable salts thereof.

43. A method for inhibiting epileptogenesis in a subject, comprising administering to a subject an effective amount of a compound such that epileptogenesis is inhibited in the subject, wherein said compound is selected from the group consisting of 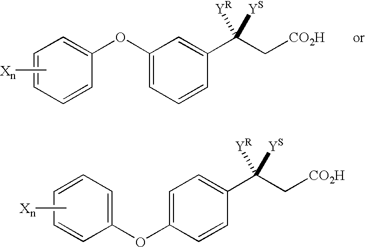
wherein each X is independently selected from the group consisting of halogen, nitro, cyano, and substituted or unsubstituted alkyl and alkoxy groups; n is an integer from 0 to 5; and one of Y^{R} and Y^{S} is a hydrogen, and the other is a substituted or unsubstituted amine; and pharmaceutically acceptable salts thereof.

44. The method according to any one of claims 42 or 43 wherein said compound is selected from the group consisting of (R)-3-amino-3-[3-(3-trifluoromethylphenoxy)phenyl]propionic acid, (S)-3-amino-3-[3-(trifluoromethylphenoxy)phenyl]propionic acid, (R)-3-amino-3-[3-(4-methylphenoxy)phenyl]propionic acid, (S)-3-amino-3-[3-(4-methylphenoxy)phenyl]propionic acid, (R)-3-amino-3-[3-(phenoxy)phenyl]propionic acid, (S)-3-amino-3-[3-(phenoxy)phenyl]propionic acid, (D)-(+)-3-amino-3-[3-(4-chlorophenoxy)phenyl]propionic acid, (L)-(−)-3-amino-3-[3-(4-chlorophenoxy)phenyl]propionic acid, (L)-(−)-3-amino-3-[3-(3,4-dichlorophenoxy)phenyl]propionic acid, (D)-(+)-3-amino-3-[3-(3,4-dichlorophenoxy)phenyl]propionic acid, 3-amino-3-(3-phenoxy)phenylpropionic acid, and pharmaceutically acceptable salts or esters thereof.

45. A method of diagnosing an epileptogenic condition in a subject comprising administering a compound selected from the group consisting of 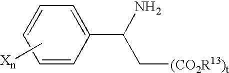
wherein R¹³ is a hydrogen, alkyl, aryl, or an organic or inorganic salt-forming cation; n is 1 to 5; t is 1 to 2; each X is independently selected from the group consisting of halogen, nitro, cyano, and substituted or unsubstituted alkyl and alkoxy groups; and pharmaceutically acceptable salts or esters thereof.

46. A method for inhibiting epileptogenesis in a subject, comprising administering to a subject an effective amount of a compound such that epileptogenesis is inhibited in the subject, wherein said compound is selected from the group consisting of 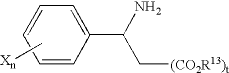
wherein R¹³ is a hydrogen, alkyl, aryl, or an organic or inorganic salt-forming cation; n is 1 to 5; t is 1 to 2; each X is independently selected from the group consisting of halogen, nitro, cyano, and substituted or unsubstituted alkyl and alkoxy groups; and pharmaceutically acceptable salts or esters thereof.

47. The method according to any one of claims 45 or 46 wherein said compound is selected from the group consisting of 3-amino-3-(4-nitrophenyl)propionic acid, 3-amino-3-(4-methylphenyl)-2-carboxypropionic acid acid, 3-amino-3-(4-methoxyphenyl)-2-carboxypropionic acid, 3-amino-3-(4-nitrophenyl)-2-carboxypropionic acid, and pharmaceutically acceptable salts or esters thereof.

48. A method of diagnosing an epileptogenic condition in a subject comprising administering anthralinic acid or a pharmaceutically acceptable salt thereof.

49. A method for inhibiting epileptogenesis in a subject, comprising administering to a subject an effective amount of anthralinic acid such that epileptogenesis is inhibited in the subject.
